(19) United States
(12) Patent Application Publication
(10) Pub. No.: US 2017/0329893 A1
(43) Pub. Date: Nov. 16, 2017

(54) METHODS OF DETERMINING GENOMIC HEALTH RISK

(71) Applicant: Human Longevity, Inc., San Diego, CA (US)

(72) Inventors: Julia di Iulio, San Diego, CA (US); Amalio Telenti, San Diego, CA (US)

(21) Appl. No.: 15/589,503

(22) Filed: May 8, 2017

Related U.S. Application Data
(60) Provisional application No. 62/333,653, filed on May 9, 2016.
(60) Provisional application No. 62/410,783, filed on Oct. 20, 2016.

(51) Int. Cl. G06F 19/18; C12Q 1/68; G06F 19/22

(52) U.S. Cl. CPC G06F 19/18; G06F 19/22; C12Q 1/6883; C12Q 1/6886; C12Q 2600/156

(57) ABSTRACT

Described are genomic health risk metrics elaborated herein to hold significant advantages for the health care industry. The likelihood that any given GSV will be deleterious is relatively small. Since every human genome sequenced may result in several million GSVs, the advantage of a genomic health risk metric such as a tolerability score, an n-mer score, a context dependent tolerance score, or a protein tolerability score to clinicians is that it will allow them to focus on and prioritize deleterious mutations.

### CROSS-REFERENCE TO RELATED APPLICATIONS

This application claims priority to U.S. Provisional Application Ser. No. 62/333,653, filed on May 9, 2016, and U.S. Provisional Application Ser. No. 62/410,783, filed on Oct. 20, 2016, each of which is incorporated herein in its entirety.

### INCORPORATION BY REFERENCE OF TABLE SUBMITTED AS TEXT FILE VIA EFS-WEB

The instant application contains a Table, which has been submitted in ASCII format via EFS-Web and is hereby incorporated by reference in its entirety. Said ASCII copy, created on May 5, 2017 is named 49523-703-201-TABLES.txt and is 2,508,219 bytes in size.

LENGTHY TABLES
The patent application contains a lengthy table section. A copy of the table is available in electronic form from the USPTO web site (http://seqdata.uspto.gov/?pageRequest=docDetail&DocID=US20170329893A1). An electronic copy of the table will also be available from the USPTO upon request and payment of the fee set forth in 37 CFR 1.19(b)(3).

### BACKGROUND

There have been several recent large-scale efforts to gain insight into both common and rare human genetic variation. Historically, these efforts utilized two principal analytical methods to gather genetic information in large scale: high-density microarrays and whole exome sequencing. More recently, technological advances have allowed for the large-scale sequencing of the whole human genome.

Most studies have generated population-based information on human diversity using low to intermediate coverage of the genome (4× to 20× sequencing depth). The highest coverage (30× or greater) has been reported for the recent sequencing of 1,070 Japanese subjects, 129 trios from the 1000 Genome Project, and 909 Icelandic subjects. This shift in paradigm is only made stronger by the recent release of the Illumina HiseqX-Ten, which allows the sequencing of up to 160 genomes at 30× mean depth in 3-day cycles, at an average cost of $1,000 to $2,000 per genome.

These advances create new complications for the health care industry and health professionals. A whole genome sequence from an individual can possess several million nucleotide variations when compared to a reference genome. While, it is well appreciated that many different gene and nucleotide variants can have a significant impact on the risk to an individual's overall health, a significant problem arises when a health care worker is presented with a previously unannotated genetic mutation. This disclosure describes a novel method to determine the impact that any given nucleotide variation has on an individual's overall health risk.

### SUMMARY

The genomic health risk metrics elaborated herein hold significant advantages for the health care industry. The likelihood that any given genomic sequence variant (GSV) will be deleterious is relatively small. Since every human genome sequenced may result in several million GSVs, the advantage of a health risk metric such as a tolerability score, an n-mer score, a context dependent tolerance score, or a protein tolerability score to clinicians is that it will allow them to focus on and prioritize deleterious mutations. Thus, the methods, systems and media of this disclosure solve significant problems that were created by virtue of advances in DNA sequencing and analysis. The methods described herein also describe a functional genomic sequencing assay that improves upon and is more efficient then previous methods such as whole-genome sequencing and exosome sequencing. The functional genomic sequencing assay described herein is allows targeted sequencing or analysis of GSV increasing the efficiency and reducing the cost of such analysis. This method is superior to other methods such as exosome sequencing in that it takes into account GSVs that occur in non-coding regions, and, thus, allows for greater sensitivity and accuracy of nucleic acid analysis.

In certain embodiments, described herein, is a method of identifying a relative genomic health risk of a genomic sequence variant in the DNA sequence of an individual, the method comprising: determining at least one genomic sequence variant in the DNA sequence of the individual; wherein the genomic sequence variant is a difference of at least one nucleotide in the individual when compared to a corresponding position in a reference genome; and comparing the at least one genomic sequence variant of the individual to a tolerability score at a corresponding position within x nucleotides of a genetic element, wherein the tolerability score comprises a function of a nucleotide variation score and an allele proportion score, wherein the nucleotide variation score is the variance observed in a plurality of genomes at the corresponding position, and the allele proportion score is the proportion of genomic variants that exceeds an incidence of 0.0001 in the plurality of genomes at the corresponding position. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, the nucleotide variation score is normalized. In certain embodiments, the genetic element is selected from any one or more of a gene promoter, gene enhancer, transcriptional start site, splice donor site, splice acceptor site, polyadenylation site, start codon, stop codon, exon/intron boundary, intron sequence, and an exon sequence, TFBS, protein domain, non-coding RNA and a regulatory element. In certain embodiments, the genomic sequence variant is within 500 nucleotides of the genetic element.

In another embodiment, described herein, is a method of identifying a relative genomic health risk of a genomic sequence variant in the DNA sequence of an individual, the method comprising: determining at least one genomic sequence variant in the DNA sequence of the individual; wherein the genomic sequence variant is a difference of at least one nucleotide in the individual when compared to a corresponding position in a reference genome; and determining an n-variant score for the at least one genomic sequence variant, wherein the n-variant score comprises a function of a count score and an allele frequency score, wherein the count score is the ratio of the number of times any genomic sequence variant occurs in a unique sequence of n-nucleotides in length in the plurality of genomes to the number of times that the unique sequence of n-nucleotides in length occurs in the reference genome, and the allele frequency score is the frequency of the proportion of genomic sequence variants that are fixed in the population, at an allele frequency greater than 0.0001 in the plurality of genomes. In certain embodiments, the unique sequence of n-nucleotides in length is greater than 3 nucleotides. In certain embodiments, the unique sequence of n-nucleotides in length is less than 100 nucleotides. In certain embodiments, the unique sequence of n-nucleotides in length is 7 nucleotides. In certain embodiments, the genomic sequence variant occurs in the center of the unique sequence of n-nucleotides. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes.

In another embodiment, described herein, is a method of identifying a relative genomic health risk of a genomic sequence variant of an individual, the method comprising: determining at least one genomic sequence variant in a DNA sequence of the individual; wherein the genomic sequence variant is a difference of at least one nucleotide in the individual when compared to a corresponding position in a reference genome; and determining if the at least one genomic sequence variant occurs within a region with a low context dependent tolerance score, wherein the context dependent tolerance score comprises a function of an observed context dependent tolerance score and an expected context dependent tolerance score, wherein the expected context dependent tolerance score is the overall probability to vary of a unique sequence of n-nucleotides in length in a certain region of x nucleotides in length in a plurality of genomes, and the observed context dependent tolerance score is a number of genomic sequence variants in a certain region of x nucleotides in length actually observed and fixed in the plurality of genomes as a function of a length of the region. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, the context dependent tolerance score comprises subtracting the expected context dependent tolerance score from the observed context dependent tolerance score.

In another embodiment, described herein, is a method of identifying a relative genomic health risk of a genomic sequence variant of an individual, the method comprising: determining at least one genomic sequence variant in a DNA sequence of the individual; wherein the genomic sequence variant is a difference of at least one nucleotide in the individual when compared to a corresponding position in a reference genome; determining if the at least one genomic sequence variant causes an amino acid variant in an expressed protein, wherein the amino acid variant is a difference of at least one amino acid when compared to a reference genome; and comparing the amino acid variant to a protein tolerability score at a corresponding position within a defined protein class, wherein the protein tolerability score comprises a diversity score, missense score, and a protein allele frequency score, wherein the diversity score is a normalized diversity metric, the missense score is the variance observed in a plurality of genomes at the corresponding position which leads to an amino acid mutation, and the protein allele frequency score is the proportion of genomic variants that leads to an amino acid variant that exceeds an incidence of 0.0001 in the plurality of genomes at the corresponding position. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, the defined protein class is selected from any one or more of a kinase, a phosphatase, a tyrosine kinase, a serine/threonine kinase, a G protein coupled receptor (GPCR), a nuclear hormone receptor, an acetylase, a chaperone, a protease, a serine protease, and a transcription factor. In certain embodiments, the diversity metric is a Shannon entropy, a Simpson diversity index, or a Wu-Kabat variability coefficient.

In another embodiment, described herein, is a non-transitory computer-readable storage media encoded with a computer program including instructions executable by a processor to create a program to identify a relative genomic health risk of a genomic sequence variant of an individual comprising: a DNA sequence for the individual; a software module to determine at least one genomic sequence variant in the DNA sequence of the individual; wherein the genomic sequence variant is a difference of at least one nucleotide in the individual when compared to a corresponding position in a reference genome; and a software module to compare the at least one genomic sequence variant of the individual to a tolerability score at a corresponding position within x-nucleotides of a genetic element, wherein the tolerability score comprises a function of a nucleotide variation score and an allele proportion score, wherein the nucleotide variation score is the variance observed in a plurality of genomes at the corresponding position, and the allele proportion score is the proportion of genomic variants that exceeds an incidence of 0.0001 in the plurality of genomes at the corresponding position. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, the nucleotide variation score is normalized to the size of the genetic element. In certain embodiments, the genetic element is selected from any one or more of a gene promoter, gene enhancer, transcriptional start site, splice donor site, splice acceptor site, polyadenylation site, start codon, stop codon, exon/intron boundary, intron sequence, and an exon sequence. In certain embodiments, the genomic sequence variant is within 50 nucleotides of the genetic element. In certain embodiments, the genomic sequence variant is within 500 nucleotides of the genetic element.

In another embodiment, described herein, is a non-transitory computer-readable storage media encoded with a computer program including instructions executable by a processor to create a program to identify a relative genomic health risk of a genomic sequence variant of an individual comprising: a DNA sequence for the individual; a software module to determine at least one genomic sequence variant in the DNA sequence of the individual; wherein the genomic sequence variant is a difference of at least one nucleotide in the individual when compared to a corresponding position in a reference genome in a unique sequence of n nucleotides in length; and a software module to determine an n-variant score for the at least one genomic sequence variant, wherein the n-variant score is comprises a function of a count score and an allele frequency score, wherein the count score is the ratio of the number of times any genomic sequence variant occurs in a unique sequence of n-nucleotides in length in the plurality of genomes to the number of times that the unique sequence of n-nucleotides in length occurs in the reference genome, and the allele frequency score is the frequency of the proportion of genomic sequence variants that are fixed in the population, at an allele frequency greater than 0.0001 in the plurality of genomes. In certain embodiments, the unique sequence of n-nucleotides in length is greater than 4 nucleotides. In certain embodiments, the unique sequence of n-nucleotides in length is less than 100 nucleotides. In certain embodiments, the unique sequence of n-nucleotides in length is 7 nucleotides. In certain embodiments, the genomic sequence variant occurs in the center of the unique sequence of n-nucleotides. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes.

In another embodiment, described herein, is a non-transitory computer-readable storage media encoded with a computer program including instructions executable by a processor to create a program to identify a relative genomic health risk of a genomic sequence variant of an individual comprising: a DNA sequence for the individual; a software module to determine at least one genomic sequence variant in a DNA sequence of the individual; wherein the genomic sequence variant is a difference of at least one nucleotide in the individual when compared to a corresponding position in a reference genome; and a software module to determine if the at least one genomic sequence variant occurs within a region with a low context dependent tolerance score, wherein the context dependent tolerance score comprises a function of an observed context dependent tolerance score and an expected context dependent tolerance score, wherein the expected context dependent tolerance score is the overall probability to vary of a unique sequence of n-nucleotides in length in a certain region of x nucleotides in length actually observed and fixed in a plurality of genomes, and the observed context dependent tolerance score is a number of genomic sequence variants in a certain region of x nucleotides in length actually observed in the plurality of genomes. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, the context dependent tolerance score comprises subtracting the expected context dependent tolerance score from the observed context dependent tolerance score.

In another embodiment, described herein, is a non-transitory computer-readable storage media encoded with a computer program including instructions executable by a processor to create a program to identify a relative genomic health risk of a genomic sequence variant of an individual comprising: a DNA sequence for the individual; a software module to determine at least one genomic sequence variant in a DNA sequence of the individual; wherein the genomic sequence variant is a difference of at least one nucleotide in the individual when compared to a corresponding position in a reference genome; a software module to determine if the at least one genomic sequence variant causes an amino acid variant in an expressed protein, wherein the amino acid variant is a difference of at least one amino acid when compared to a reference genome; and a software module to compare the amino acid variant to a protein tolerability score at a corresponding position within a defined protein class, wherein the protein tolerability score comprises a diversity score, missense score, and a protein allele frequency score, wherein the diversity score is a normalized diversity metric, the missense score is the variance observed in a plurality of genomes at the corresponding position which leads to an amino acid mutation, and the protein allele frequency score is the proportion of genomic variants that leads to an amino acid variant that exceeds an incidence of 0.0001 in the plurality of genomes at the corresponding position. In certain embodiments, the plurality of genomes is at least 10,000 genomes. In certain embodiments, the plurality of genomes is at least 100,000 genomes. In certain embodiments, the DNA sequence comprises at least 100,000 nucleotides. In certain embodiments, the DNA sequence comprises at least 90% of human haploid genome. In certain embodiments, at least 100 genomic sequence variants are determined in the DNA sequence of the individual. In certain embodiments, the reference genome is generated from at least 10,000 individual genomes. In certain embodiments, the reference genome is generated from at least 100,000 individual genomes. In certain embodiments, the genomic sequence variant is an insertion, a deletion, or a translocation. In certain embodiments, the genomic sequence variant is a point mutation. In certain embodiments, defined protein class is selected from any one or more of a kinase, a phosphatase, a tyrosine kinase, a serine/threonine kinase, a G protein coupled receptor (GPCR), a nuclear hormone receptor, an acetylase, a chaperone, a protease, a serine protease, and a transcription factor. In certain embodiments, the diversity metric is a Shannon entropy, a Simpson diversity index, or a Wu-Kabat variability coefficient. In another embodiment, described herein, is a method of creating a genomic health risk database comprising: populating a database with a tolerability score value for each of a plurality of positions in a genome; wherein the tolerability score is determined for each of the plurality of positions in the genome within x nucleotides of a genetic element, wherein the tolerability score comprises a function of a nucleotide variation score and an allele proportion score; wherein the nucleotide variation score is the nucleotide variance observed in a plurality of genomes at each of the plurality of positions in the genome, and the allele proportion score is the proportion of genomic variants that exceed an incidence of 0.0001 in the plurality of genomes at each of the plurality of positions in the genome. In certain embodiments, the plurality of genomes is at least 10,000 genomes. In certain embodiments, the plurality of genomes is at least 100,000 genomes. In certain embodiments, the nucleotide variance is an insertion, a deletion, or a translocation. In certain embodiments, the nucleotide variance is a point mutation. In certain embodiments, the nucleotide variation score is normalized to the size of the genetic element. In certain embodiments, the plurality of positions is greater than 1,000. In certain embodiments, the genetic element is selected from any one or more of a gene promoter, gene enhancer, transcriptional start site, splice donor site, splice acceptor site, polyadenylation site, start codon, stop codon, exon/intron boundary, intron sequence, and an exon sequence. In certain embodiments, the tolerability score is determined for each of a plurality of positions in the genome within 500 nucleotides of the genetic element.

In another embodiment, described herein, is a method of creating a genomic health risk database comprising: populating a database with an n-variant score value for each of a plurality of positions in a genome; wherein the n-variant score is determined for each of the plurality of positions in the genome, wherein the n-variant score comprises a function of a count score and an allele frequency score; wherein the count score is the ratio of the number of times any genomic sequence variant occurs in a unique sequence of n-nucleotides in length in the plurality of genomes compared to a reference genome to the number of times that the unique sequence of n-nucleotides in length occurs in the reference genome, and the allele frequency score is the frequency of the proportion of genomic sequence variants that are fixed in the population, in the plurality of genomes for each of the plurality of positions in the genome. In certain embodiments, the unique sequence of n-nucleotides in length is greater than 4 nucleotides. In certain embodiments, the unique sequence of n-nucleotides in length is less than 100 nucleotides. In certain embodiments, the unique sequence of n-nucleotides in length is 7 nucleotides. In certain embodiments, the genomic sequence variant occurs in the center of the unique sequence of n-nucleotides. In certain embodiments, the plurality of genomes is at least 10,000 genomes. In certain embodiments, the plurality of genomes is at least 100,000 genomes.

A method of creating a genomic health risk database comprising: populating a database with a context dependent tolerance score for each of a plurality of regions in a genome; wherein the context dependent tolerance score comprises a function of an observed context dependent tolerance score and an expected context dependent tolerance score; wherein the expected context dependent tolerance score is the overall probability to vary of a unique sequence of n-nucleotides in length in a certain region of x nucleotides in length actually observed and fixed in a plurality of genomes, and the observed context dependent tolerance score is a number of genomic sequence variants in a certain region of x nucleotides in length actually observed in the plurality of genomes. In certain embodiments, the plurality of genomes is at least 10,000 genomes. In certain embodiments, the plurality of genomes is at least 100,000 genomes. In certain embodiments, the genomic sequence variant is an insertion, a deletion, or a translocation. In certain embodiments, the genomic sequence variant is a point mutation. In certain embodiments, the context dependent tolerance score comprises subtracting the expected context dependent tolerance score from the observed context dependent tolerance score.

In another embodiment, described herein, is a method of creating a genomic health risk database comprising: populating a database with a protein tolerability score value for each of a plurality of positions in a genome; wherein the protein tolerability score is determined for each of the plurality of positions in the genome, wherein the protein tolerability score comprises a function of a diversity score, missense score, and a protein allele frequency score; wherein the diversity score is a normalized diversity metric, the missense score is the variance observed in a plurality of genomes at each of the plurality of positions in the genome which leads to an amino acid variant, and the protein allele frequency score is the proportion of genomic variants that leads to an amino acid variant at each of the plurality of positions in the genome. In certain embodiments, the plurality of genomes is at least 10,000 genomes. In certain embodiments, the plurality of genomes is at least 100,000 genomes. In certain embodiments, the is an insertion, a deletion, or a translocation. In certain embodiments, the genomic sequence variant is a point mutation. In certain embodiments, the defined protein class is selected from any one or more of a kinase, a phosphatase, a tyrosine kinase, a serine/threonine kinase, a G protein coupled receptor (GPCR), a nuclear hormone receptor, an acetylase, a chaperone, a protease, a serine protease, and a transcription factor. In certain embodiments, the diversity metric is a Shannon entropy, a Simpson diversity index, or a Wu-Kabat variability coefficient.

In another embodiment, described herein, is a genomic assay comprising a plurality of polynucleotides bound to a substrate, wherein each of the plurality of polynucleotides possess a sequence corresponding to a genomic locus, wherein a sequence corresponding to the genomic locus possesses a tolerability score below 0.1, wherein the tolerability score comprises a function of a nucleotide variation score and an allele proportion score, wherein the nucleotide variation score is the variance observed in a plurality of genomes at the corresponding position, and the allele proportion score is the proportion of genomic variants that exceeds an incidence of 0.0001 in the plurality of genomes at the corresponding position. In certain embodiments, the plurality of genomes is at least 10,000 genomes. In certain embodiments, the plurality of polynucleotides is at least 1,000 polynucleotides. In certain embodiments, the plurality of polynucleotides is at least 10,000 polynucleotides. In certain embodiments, the plurality of polynucleotides comprises at least 4,000 distinct nucleotide sequences. In certain embodiments, the plurality of polynucleotides comprises at least 4,000 distinct nucleotide sequences. In certain embodiments, the plurality of polynucleotides comprises at least 8,000 distinct nucleotide sequences. In certain embodiments, the plurality of polynucleotides are covalently bound to the substrate. In certain embodiments, the plurality of polynucleotides are covalently bound to the substrate at their 5 prime ends. In certain embodiments, the plurality of polynucleotides are covalently bound to the substrate at their 3 prime ends. In certain embodiments, the plurality of polynucleotides further comprises a fluorescent molecule. In certain embodiments, the plurality of polynucleotides further comprises a fluorescent dye. In certain embodiments, the substrate comprises glass. In certain embodiments, the substrate comprises silicon.

In another embodiment, described herein, is a genomic assay comprising a plurality of polynucleotides bound to a substrate, wherein each of the plurality of polynucleotides possess a sequence corresponding to a genomic locus, wherein a sequence corresponding to the genomic locus possesses an n-variant score below 0.05 wherein the n-variant score comprises a function of a count score and an allele frequency score, wherein the count score is the ratio of the number of times any genomic sequence variant occurs in a unique sequence of n-nucleotides in length in the plurality of genomes to the number of times that the unique sequence of n-nucleotides in length occurs in the reference genome, and the allele frequency score is the frequency of the proportion of genomic sequence variants that are fixed in the population, at an allele frequency greater than 0.0001, in the plurality of genomes. In certain embodiments, the plurality of genomes is at least 10,000 genomes. In certain embodiments, the plurality of polynucleotides is at least 1,000 polynucleotides. In certain embodiments, the plurality of polynucleotides is at least 10,000 polynucleotides. In certain embodiments, the plurality of polynucleotides comprise at least 4,000 distinct nucleotide sequences. In certain embodiments, the plurality of polynucleotides comprise at least 4,000 distinct nucleotide sequences. In certain embodiments, the plurality of polynucleotides comprise at least 8,000 distinct nucleotide sequences. In certain embodiments, the plurality of polynucleotides are covalently bound to the substrate. In certain embodiments, the plurality of polynucleotides are covalently bound to the substrate at their 5 prime ends. In certain embodiments, the plurality of polynucleotides are covalently bound to the substrate at their 3 prime ends. In certain embodiments, the plurality of polynucleotides further comprise a fluorescent molecule. In certain embodiments, the plurality of polynucleotides further comprise a fluorescent dye. In certain embodiments, the substrate comprises glass. In certain embodiments, the substrate comprises silicon.

In another embodiment, described herein, is a genomic assay comprising a plurality of polynucleotides bound to a substrate, wherein each of the plurality of polynucleotides possess a sequence corresponding to a genomic locus, wherein a sequence corresponding to the genomic locus possesses a low context dependent tolerance score, wherein the context dependent tolerance score comprises a function of an observed context dependent tolerance score and an expected context dependent tolerance score, wherein the expected context dependent tolerance score is the overall probability to vary of a unique sequence of n-nucleotides in length in a certain region of x nucleotides in length actually observed and fixed in a plurality of genomes, and the observed context dependent tolerance score is a number of genomic sequence variants in a certain region of x nucleotides in length actually observed in the plurality of genomes. In certain embodiments, the context dependent tolerance score comprises subtracting the expected context dependent tolerance score from the observed context dependent tolerance score. In certain embodiments, the plurality of genomes is at least 10,000 genomes. In certain embodiments, plurality of polynucleotides is at least 1,000 polynucleotides. In certain embodiments, plurality of polynucleotides is at least 10,000 polynucleotides. In certain embodiments, the plurality of polynucleotides comprise at least 4,000 distinct nucleotide sequences. In certain embodiments, the plurality of polynucleotides comprise at least 4,000 distinct nucleotide sequences. In certain embodiments, the plurality of polynucleotides comprise at least 8,000 distinct nucleotide sequences. In certain embodiments, the plurality of polynucleotides are covalently bound to the substrate. In certain embodiments, the plurality of polynucleotides are covalently bound to the substrate at their 5 prime ends. In certain embodiments, the plurality of polynucleotides are covalently bound to the substrate at their 3 prime ends. In certain embodiments, the plurality of polynucleotides further comprise a fluorescent molecule. In certain embodiments, the plurality of polynucleotides further comprise a fluorescent dye. In certain embodiments, the substrate comprises glass. In certain embodiments, the substrate comprises silicon.

Any of the methods of this disclosure can be used to determine a section of the genome for targeted sequencing, resequencing, or SNP analysis.

In another embodiment, described herein, is a functional genomic assay comprising: identifying a presence of at least one genomic sequence variant in the nucleic acid sequence of an individual; determining if the at least one genomic sequence variant occurs in a highly conserved genomic region; the highly conserved genomic region having an observed context dependent tolerance score greater than an expected context dependent tolerance score, wherein the expected context dependent tolerance score is the probability to vary of a unique nucleic acid sequence of n-nucleotides in length in a certain region of x nucleotides in length in a plurality of genomes, and the observed context dependent tolerance score is a number of genomic sequence variants in the certain region of x nucleotides in length actually observed in the plurality of genomes. In certain embodiments, the nucleic acid sequence comprises a DNA sequence. In certain embodiments, the DNA sequence comprises a nuclear DNA sequence. In certain embodiments, the plurality of genomes is at least 10,000 genomes. In certain embodiments, the nucleic acid sequence comprises at least 100,000 nucleotides. In certain embodiments, the functional genomic assay comprises identifying the presence of at least 10 genomic sequence variants. In certain embodiments, the at least one genomic sequence variant comprises at least one of an insertion, a deletion, and a translocation. In certain embodiments, the at least one genomic sequence variant comprises a single nucleotide polymorphism. In certain embodiments, n equals 7. In certain embodiments, x is between 400 and 600. In certain embodiments, the functional genomic assay comprises determining if the at least one genomic sequence variant is in a non-coding genomic region that is highly conserved. In certain embodiments, the at least one genomic sequence variant is in a non-coding highly conserved genomic region within 1,000 base pairs of a known disease-associated gene. In certain embodiments, the highly conserved genomic region is a genomic region corresponding to a most conserved 1^{st} percentile of all genomic regions. In certain embodiments, the observed context dependent tolerance score is at least 10% greater than an expected context dependent tolerance score. In certain embodiments, at least one of the at least one genomic sequence variant in a non-coding genomic region that is highly conserved is selected from the list consisting of rs587780751, rs745366624, rs777251123, rs778796405, rs774531501, rs587776927, rs768823171, rs749303140, rs376829288, rs750530042, rs587776558, rs372686280, rs111812550, rs143144732, rs193922699, rs750180293, rs398122808, rs757171524, rs773306994, rs773306994, rs372418954, rs762425885, rs397516031, rs397516022, rs730880592, rs730880592, rs397516020, rs397516020, rs373746463, rs373746463, rs373746463, rs387906397, rs387906397, rs587782958, rs730880718, rs730880667, rs113358486, rs111683277, rs112917345, rs730880691, rs397515916, rs730880690, rs111437311, rs397515903, rs727503201, rs112999777, rs397515897, rs727503204, rs397515893, rs397515891, rs587776699, rs587776700, rs376395543, rs748486465, rs149712664, rs199683937, rs144637717, rs587776644, rs730880296, rs397515322, rs558721552, rs531105836, rs587777262, rs267607302, rs387907354, rs398123750, rs727503988, rs587783714, rs148622862, rs763991428, rs761780097, rs770204470, rs387906521, rs387906520, rs79367981, rs749160734, rs587776708, rs587776708, rs34086577, rs199959804, rs587777290, rs386834170, rs386834169, rs144077391, rs386834164, rs386834166, rs770093080, rs587777374, rs45517105, rs45517105, rs45488500, rs45517289, rs45517289, rs137854118, rs45517358, rs189077405, rs515726118, rs386833742, rs386833739, rs755127868, rs200655247, rs376023420, rs747351687, rs113690956, rs376281637, rs765390290, rs773401248, rs61750189, rs530975087, rs201978571, rs267604791, rs80358116, rs80358116, rs273899695, rs80358011, rs80358011, rs80358051, rs730880267, rs63751296, rs63750707, rs776442328, rs776820510, rs72653165, rs72667012, rs72667008, rs527398797, rs587780009, rs587776658, rs587782018, rs745620135, rs372651309, rs556992558, rs137853932, rs200253809, rs386833901, rs770882876, rs750550558, rs397507554, rs730880306, rs201613240, rs147952488, rs770241629, rs373494631, rs397517741, rs386833856, rs559854357, rs371496308, rs539645405, rs187510057, rs41298629, rs536892777, rs747330606, rs748559929, rs770277446, rs201685922, rs767245071, rs730882032, rs587776525, rs398123358, rs72659359, rs137853943, rs267607709, rs267607710, rs766168993, rs775288140, rs780041521, rs145564018, rs775456047, rs587776879, rs540289812, rs745832717, rs745915863, rs386833418, rs199422309, rs431905514, rs587784059, rs748086984, rs386833492, rs199988476, rs281865166, rs587776515, rs397518439, rs193922258, rs142637046, rs73717525, rs145483167, rs587777285, rs747737281, rs183894680, rs116735828, rs574673404, rs386833563, rs768154316, rs111033661, rs755363896, rs368953604, rs180177319, rs148049120, rs150676454, rs372655486, rs373842615, rs763389916, rs118203419, rs515726232, rs312262809, rs312262804, rs281865349, rs281865338, rs281865337, rs281865334, rs281865336, rs281865336, rs62638626, rs62638627, rs587784423, rs113951193, rs281874765, rs104886349, rs398123247, rs74315277, rs200346587, rs398122908, rs727503036, rs397515747, and rs587776734. In certain embodiments, at least one of the at least one genomic sequence variant in a non-coding region that is highly conserved is selected from the list consisting of rs778796405, rs8177982, rs376829288, rs4253196, rs750180293, rs757171524, rs727503201, rs397515893, rs587776699, rs397516083, rs201078659, rs750425291, rs558721552, rs531105836, rs200782636, rs752197734, rs3093266, rs34086577, rs199959804, rs144077391, rs386834164, rs386834166, rs189077405, rs746701685, rs386833721, rs376023420, rs761146008, rs765390290, rs72648337, rs527398797, rs367567416; rs372651309, rs200253809, rs193922837, rs761737358, rs113994173, rs559854357, rs111951711, rs371496308, rs368123079, rs118192239, rs41298629, and rs536892777. In certain embodiments, the functional genomic assay is for use in determining a likelihood of the individual being diagnosed with a cancer. In certain embodiments, the functional genomic assay is for use in prognosing a cancer of the individual.

In another embodiment, described herein, is a computer-implemented system comprising: a computer comprising: at least one processor, a memory, an operating system configured to perform executable instructions, and a computer program including instructions executable by the at least one processor to create a functional genomic assay application, the functional genomic assay application configured to perform the following: receiving a nucleic acid sequence of an individual; identifying a presence of at least one genomic sequence variant in the nucleic acid sequence of the individual; and determining if the at least one genomic sequence variant occurs in a highly conserved genomic region, the highly conserved genomic region having an observed context dependent tolerance score greater than an expected context dependent tolerance score, wherein the expected context dependent tolerance score is the probability to vary of a unique nucleic acid sequence of n-nucleotides in length in a certain region of x nucleotides in length in a plurality of genomes, and the observed context dependent tolerance score is a number of genomic sequence variants in the certain region of x nucleotides in length actually observed in the plurality of genomes. The nucleic acid sequence may comprise a DNA sequence and in some cases, the DNA sequence comprises a nuclear DNA sequence. In some cases, the plurality of genomes is at least 10,000 genomes. In some cases, the nucleic acid sequence comprises at least 100,000 nucleotides. The functional genomic assay may comprise identifying the presence of at least 10 genomic sequence variants. In some cases, the at least one genomic sequence variant comprises at least one of an insertion, a deletion, and a translocation. In some cases, the at least one genomic sequence variant comprises a single nucleotide polymorphism. In particular embodiments of the functional genomic assay n equals 7. In some embodiments of the functional genomic assay x is between 400 and 600. The functional genomic assay may comprise determining if the at least one genomic sequence variant is in a non-coding highly conserved genomic region. In some cases, the at least one genomic sequence variant is in a non-coding highly conserved genomic region within 2 megabases of a known disease-associated gene. In some cases, the highly conserved genomic region is a genomic region corresponding to a most conserved 1st percentile of all genomic regions. In some cases, the observed context dependent tolerance score is at least 10% greater than an expected context dependent tolerance score. In various cases, at least one of the at least one genomic sequence variant in a non-coding genomic region that is highly conserved is selected from the list consisting of rs587780751, rs745366624, rs777251123, rs778796405, rs774531501, rs587776927, rs768823171, rs749303140, rs376829288, rs750530042, rs587776558, rs372686280, rs111812550, rs143144732, rs193922699, rs750180293, rs398122808, rs757171524, rs773306994, rs773306994, rs372418954, rs762425885, rs397516031, rs397516022, rs730880592, rs730880592, rs397516020, rs397516020, rs373746463, rs373746463, rs373746463, rs387906397, rs387906397, rs587782958, rs730880718, rs730880667, rs113358486, rs111683277, rs112917345, rs730880691, rs397515916, rs730880690, rs111437311, rs397515903, rs727503201, rs112999777, rs397515897, rs727503204, rs397515893, rs397515891, rs587776699, rs587776700, rs376395543, rs748486465, rs149712664, rs199683937, rs144637717, rs587776644, rs730880296, rs397515322, rs558721552, rs531105836, rs587777262, rs267607302, rs387907354, rs398123750, rs727503988, rs587783714, rs148622862, rs763991428, rs761780097, rs770204470, rs387906521, rs387906520, rs79367981, rs749160734, rs587776708, rs587776708, rs34086577, rs199959804, rs587777290, rs386834170, rs386834169, rs144077391, rs386834164, rs386834166, rs770093080, rs587777374, rs45517105, rs45517105, rs45488500, rs45517289, rs45517289, rs137854118, rs45517358, rs189077405, rs515726118, rs386833742, rs386833739, rs755127868, rs200655247, rs376023420, rs747351687, rs113690956, rs376281637, rs765390290, rs773401248, rs61750189, rs530975087, rs201978571, rs267604791, rs80358116, rs80358116, rs273899695, rs80358011, rs80358011, rs80358051, rs730880267, rs63751296, rs63750707, rs776442328, rs776820510, rs72653165, rs72667012, rs72667008, rs527398797, rs587780009, rs587776658, rs587782018, rs745620135, rs372651309, rs556992558, rs137853932, rs200253809, rs386833901, rs770882876, rs750550558, rs397507554, rs730880306, rs201613240, rs147952488, rs770241629, rs373494631, rs397517741, rs386833856, rs559854357, rs371496308, rs539645405, rs187510057, rs41298629, rs536892777, rs747330606, rs748559929, rs770277446, rs201685922, rs767245071, rs730882032, rs587776525, rs398123358, rs72659359, rs137853943, rs267607709, rs267607710, rs766168993, rs775288140, rs780041521, rs145564018, rs775456047, rs587776879, rs540289812, rs745832717, rs745915863, rs386833418, rs199422309, rs431905514, rs587784059, rs748086984, rs386833492, rs199988476, rs281865166, rs587776515, rs397518439, rs193922258, rs142637046, rs73717525, rs145483167, rs587777285, rs747737281, rs183894680, rs116735828, rs574673404, rs386833563, rs768154316, rs111033661, rs755363896, rs368953604, rs180177319, rs148049120, rs150676454, rs372655486, rs373842615, rs763389916, rs118203419, rs515726232, rs312262809, rs312262804, rs281865349, rs281865338, rs281865337, rs281865334, rs281865336, rs281865336, rs62638626, rs62638627, rs587784423, rs113951193, rs281874765, rs104886349, rs398123247, rs74315277, rs200346587, rs398122908, rs727503036, rs397515747, and rs587776734. In various embodiments, at least one of the at least one genomic sequence variant in a non-coding region that is highly conserved is selected from the list consisting of rs778796405, rs8177982, rs376829288, rs4253196, rs750180293, rs757171524, rs727503201, rs397515893, rs587776699, rs397516083, rs201078659, rs750425291, rs558721552, rs531105836, rs200782636, rs752197734, rs3093266, rs34086577, rs199959804, rs144077391, rs386834164, rs386834166, rs189077405, rs746701685, rs386833721, rs376023420, rs761146008, rs765390290, rs72648337, rs527398797, rs367567416; rs372651309, rs200253809, rs193922837, rs761737358, rs113994173, rs559854357, rs111951711, rs371496308, rs368123079, rs118192239, rs41298629, and rs536892777. The functional genomic assay may be for use in determining a likelihood of the individual being diagnosed with a cancer, for use in prognosing a cancer of the individual, and/or for use in determining longevity of the individual.

### BRIEF DESCRIPTION OF THE DRAWINGS

FIG. 1 illustrates a scheme, in the form of a metaprofile strategy, for determining a tolerability score for a genomic sequence variant (GSV).

FIG. 2 illustrates a scheme, in the form of a heptameric variant score strategy, for determining an n-mer score for a GSV.

FIG. 3 illustrates a scheme, in the form of a heptameric variant score expected versus observed strategy, for determining a context dependent tolerance score.

FIG. 4 illustrates a scheme, in the form of a protein tolerance score strategy, for determining a protein tolerance score for a GSV.

FIG. 5A illustrates a functional genomic scheme as applied to chromosome 1.

FIG. 5B illustrates enrichment of genetic elements by a percentile ranking of conservation.

FIG. 5C illustrates a distribution of the percentile ranking of conservation among selected genetic elements.

FIG. 6A illustrates an analysis of the relationship of mean coverage with effective genome coverage uses 100 NA12878 replicates with coverage <30×, 200 replicates with mean coverage of 30× to 40×, and 25 replicates with >40×. Vertical grey lines highlight mean target coverage of 7× and 30×. Each sequencing replica is plotted at 10× (blue) and 30× (orange) effective minimal genome coverage.

FIG. 6B illustrates an analysis of reproducibility uses NA12878 genomes at 30×-40× mean coverage (two clustering chemistries, v1 and v2, each n=100 replicas) to assess the consistency of base calling at each position in the whole genome. The analysis of reproducibility is then extended to 100 unrelated genomes (25 genomes per main ancestry group, African, European, Asian, and for 25 admixed individuals). The color bars represent degree of consistency (blue 100%, light blue >90%, orange >10-<90%, red <10%, black, no-PASS).

FIG. 6C illustrates that false positive calls are concentrated in the region of GiaB that has <90% reproducibility of base calling. False negative calls are more evenly represented across GiaB; missingness (no-PASS) represents the bulk of error.

FIG. 7A provides a genome view of a representative autosomal chromosome sequenced; Chr.1 is the longest human chromosome. Each data point represents a 1 kb window; the Y axis represents the number of SNVs per 1 kb; dark blue are high confidence windows (the overlap of GiaB high confidence regions and regions with >=90% reproducibility in NA12878 replicates); light blue are extended confidence windows outside of GiaB; pink are GiaB only (low reproducibility with current technology); grey dots are regions outside of GiaB and extended confidence regions.

FIG. 7B provides a genome view of a representative autosomal chromosome sequenced; Chr. 22 with the lowest proportion of sequenceable bases with the technology used, using the same color-coding as in FIG. 7A.

FIG. 7C provides summary statistics for all the chromosomes, using the same color-coding as in FIGS. 7A and 7B.

FIG. 8A illustrates the distribution of SNVs in selected genomic elements (genomic, protein-coding, RNA coding and regulatory elements). The genome average of 56.59 SNVs per kb is indicated by the horizontal dashed line. AE, alternative exon; AI, alternative intron; CE, constitutive exon; CI, constitutive intron; oriC, origin of replication.

FIG. 8B illustrates the metaprofiles of protein-coding genes created by aligning all elements of 6 different genomic landmarks (TSS, start codon, SD, SA, stop codon and pA) for all 10,545 genomes. The y-axis in the upper representation describes the enrichment/depletion of SNVs occurrence per position, normalized to the mean (indicated by the horizontal dashed line); the y-axis in the lower representation describes the percent of SNVs at each position with an allelic frequency higher than 1 in a 1000. The x-axis represents the distance from the genomic landmark. The vertical line indicates the genomic landmark position. The SD and SA metaprofiles highlight the strong conservation of the splice sites (upper panel) and the difference in SNV allele frequency between exons and introns (lower panel). TSS, transcription start site; SD, splice donor site; SA, splice acceptor site; and pA, poly adenylation site.

FIG. 8C illustrates the metaprofiles of transcription factor binding sites (TFBS) created by aligning all the binding sites of four transcription factors (FOXA1, STAT3, NFKB1, MAFF) for all 10,545 genomes. The y-axis describes the normalized enrichment/depletion of SNVs occurrence per position, normalized to the mean (indicated by the horizontal dashed line). The x-axis represents the distance from the 5′ end of the TFBS. The vertical lines indicate the 5′ and 3′ ends of the TFBS. TFBS, transcription factor binding site.

FIG. 9A illustrates a Metaprofile of the transition between introns and exons expressed as Tolerance Score (TS). The TS is the product of the normalized SNV distribution value by the proportion of SNVs with allele frequency >0.001 (see FIG. 3B). The exon sequence highlights the conservation of the first and second positions in codons and the tolerance to variation of the third position in codons (red). The pattern of higher tolerance to variation every third nucleotide is lost in introns. The TS is lowest at the splice donor and acceptor sites and highest in introns.

FIG. 9B illustrates the distribution of ClinVar and HGMD pathogenic SNVs (n=29,808 in SD; n=30,369 in SA metaprofiles) reflecting a significant enrichment of pathogenic variants at the sites of lowest TA. Consistently, the exon sequence highlights the enrichment for variation at the first position in codons (blue), as it results in amino acid change or truncation.

FIG. 9C illustrates the relationship of tolerance score and enrichment for pathogenic variants. Represented on x-axis are the median TS values of 1200 positions (six protein-coding landmark positions +/−100 bp) expressed in 100 bins. The y-axis presents the fold enrichment in pathogenic variants per bin. The LOESS curve fitting is represented by the solid line; the shaded area indicates the 95% confidence interval.

FIG. 9D illustrates an orthogonal assessment of the impact of variation at sites with lowest TS values. The x-axis represents a gene essentiality score (the posterior probability of intolerance to truncation). The y-axis represents the fraction of genes with a given essentiality score or lower. Purple=genes with no variation in splice donor (SD) or acceptor (SA) sites, Orange=genes with variation only in SD sites, Blue=genes with variation only in SA sites, Green=genes with variation in SD and SA sites.

FIG. 10A illustrates the SNV discovery rate for 8,137 unrelated individual genomes contributing over 150 million SNVs (blue line). The projection for discovery rates as more genomes are sequenced is represented without (dashed black line) and with correction for the empirical false discovery rate of 0.0025 (dashed orange line). The number of SNVs in dbSNP is represented by the horizontal straight grey line.

FIG. 10B illustrates the number of newly observed variants, as more individuals' sequences are determined by the ancestry background and number of participants in the study. Shown are the rates of identification of novel variants for each additional African genome (13,539 SNVs), and for each additional genome of ad-mixed individuals (10,918 SNVs). The most numerous population in the study, Europeans, contribute the lowest number of novel variants (7,215 SNVs).

FIG. 10C illustrates unmapped sequences from the analysis of 8,137 unrelated individual genomes contributing over 3.2 Mb of non-reference genome. The 4,876 unique non-reference contigs had matches in NCBI nucleotide database as human (1.89 Mb), or primate (0.189 Mb). There are contigs with human-like features that do not have a known match in databases. In addition, there are 0.82 Mb of sequence mapping to the alternate scaffolds of the hg38 assembly.

FIG. 11A shows that there is very limited overlap between human conserved regions assessed with context dependent tolerance score (CDTS) and interspecies conservation assessed with GERP. Boxes in the bar correspond to different element families. The coloring of the boxes is in the same order as the legend CDTS, context-dependent tolerance score. GERP, Genomic Evolutionary Rate Profiling.

FIG. 11B shows that there is very limited overlap between human conserved regions assessed with CDTS and interspecies conservation assessed with GERP. Length of the first percentile regions of CDTS, GERP and the overlap region of CDTS and GERP. Bins without GERP score, due to insufficient multiple species alignments in the region, were not considered in the ranking process. This explains the total length difference between the first percentile regions of CDTS and GERP. CDTS, context-dependent tolerance score. GERP, Genomic Evolutionary Rate Profiling.

FIG. 11C shows element family composition in the first 10 percentile regions of CDTS (the bar labelled as “CTDS 1-10^{th}”), GERP (“GERP 1-10^{th}”) and the overlap region (“Intersection”) shows that there is very limited overlap between human conserved regions assessed with CDTS and interspecies conservation assessed with GERP. CDTS, context-dependent tolerance score. GERP, Genomic Evolutionary Rate Profiling.

FIG. 11D shows length of the first 10 percentile regions of CDTS, GERP and the overlap region of CDTS and GERP. CDTS, context-dependent tolerance score. GERP, Genomic Evolutionary Rate Profiling.

FIG. 12A shows shared conservation of genes and cis or distal regulatory elements. Coordination of cis-elements. Each genomic bin within 15 kb of a gene (cis) is attributed the essentiality score of the closest gene. The median essentiality score of the closest genes is depicted on the Y-axis for each genomic element family throughout the CDTS spectrum (X-axis). The grey horizontal dashed line represents the median gene essentiality score genome-wide (0.028). Coordination of hypothetical gene-distal enhancer pairs. A scheme of a chromatin loop with the gene-enhancer pair is depicted in the right panel. Gene-enhancer pairs brought together by chromatin looping were assessed. The X-axis represent the enhancers median CDTS and Y-axis the essentiality of the associated gene. CDTS, context-dependent tolerance score. CDTS, context-dependent tolerance score.

FIG. 12B shows shared conservation of genes and cis or distal regulatory elements. Distal coordination of anchor regions. A chromatin loop is depicted in the right panel. The median CDTS is extracted for each anchor region and binned in percentile slices. The X- and Y-axes indicate the median CDTS values for the upstream and downstream anchor regions, respectively. The anchor regions surrounding a loop share CDTS values. The whiskers extend from the 10th to the 90th percentiles of the data. The box spans the interquartile range. Outliers are not displayed. CDTS, context-dependent tolerance score.

FIG. 12C shows shared conservation of genes and cis or distal regulatory elements. Coordination of hypothetical gene-distal enhancer pairs. A scheme of a chromatin loop with the gene-enhancer pair is depicted in the right panel. Gene-enhancer pairs brought together by chromatin looping were assessed. The X-axis represent the enhancers median CDTS and Y-axis the essentiality of the associated gene. CDTS, context-dependent tolerance score.

FIG. 13A shows the distribution of pathogenic variants across the genome. The distribution of pathogenic variants across the different percentile slices identifies a strong enrichment at lower CDTS percentiles. The relative enrichment is calculated with regards to the 100^{th} percentile. Protein-coding pathogenic variants are shown in dark blue; non-coding pathogenic variants in red. The total number of pathogenic variants are N=117,257 protein-coding and N=12,996 non-coding variants. Exonic non-coding (e.g., lincRNA) are not displayed here as it contained only a very limited number of annotated pathogenic variants (N=514). CDTS, context-dependent tolerance score. Vs, versus.

FIG. 13B shows the distribution of pathogenic variants across the genome. Non-coding pathogenic variants associated with Mendelian traits. The total number of Mendelian associated non-coding pathogenic variants is N=550. Pathogenic variants are enriched at the lowest percentiles. CDTS, context-dependent tolerance score. Vs, versus.

FIG. 14A shows the complementarity of scores for non-coding variants. The enrichment of pathogenic variant detection, as compared to random, is displayed at different percentile thresholds for Eigen non-coding, CDTS, CADD as well as for the union of the three metrics.

FIG. 14B shows the complementarity of scores for non-coding variants. The barplot displays, at different percentile thresholds, the fraction of pathogenic variants identified exclusively by only one of the metrics. The Venn diagram displayed on top of each percentile threshold shows the overlap of pathogenic variant.

FIGS. 15A and 15B Shows performance and complementarity of CDTS and other scores for non-coding variants. A. Receiver operating characteristic (ROC) curves for CDTS and six additional scores. The inset figure highlights the performance at the lowest false positive rate (x axis), which represents the most relevant segment for variant prioritization. B. Number of pathogenic variants identified by each metric at their first percentile. The darker hue represents the subset that is uniquely identified by a single metric. CDTS contributes a significant number of uniquely identified variants, demonstrating its complementarity to the other metrics. The plots and percentiles are computed on 1,369 non-coding pathogenic variants and over 5 million common variants (af>0.05) as controls. CDTS, context-dependent tolerance score. CADD, combined annotation dependent depletion. GERP, genomic evolutionary rate profiling.

FIG. 16A illustrates the difference between a principal isoform (PI) and non-principal isoform (NPI)

FIG. 16B show the characteristics of exon-intron junctions in terms of tolerance to variation as assessed by metaprofiling for principal isoforms.

FIG. 16C show the characteristics of exon-intron junctions in terms of tolerance to variation as assessed by metaprofiling for non-principal isoforms.

FIG. 17 shows a depiction of novel obesity related genomic sequence variants.

FIG. 18 shows a non-limiting example of a digital processing device; in this case, a device with one or more CPUs, a memory, a communication interface, and a display. The devices and connectivity can be used to deliver reports accessible by health care professionals. The reports can be generated by any of the methods of the current disclosure.

### DETAILED DESCRIPTION

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein “genomic sequence variant” refers to any nucleotide difference in an individual's genome sequence compared to a reference genome. The variant can be a single nucleotide variant (SNV or SNP), insertion or deletion (Indel), or translocation. In certain embodiments, the indel comprises more than a single nucleotide. In certain embodiments, a genomic sequence variant excludes mitochondrial deoxyribonucleic acid (DNA) sequences. In certain embodiments, a genomic sequence variant excludes variants found on either of the non-autosomal human X or Y chromosomes. In certain embodiments, the genomic sequence variant is a human genomic sequence variant.

As used herein “reference genome” refers to any standard publicly available reference genome, for example GRCh38, the Genome Reference Consortium human genome (build 38). Alternatively, the reference genome can be one that is constructed de novo from sequencing a plurality of genomes. In certain embodiments, the plurality of genomes is greater than 10,000 different genomes. In certain embodiments, the plurality of genomes is greater than 100,000 different genomes.

### Nucleic Sequences

Described herein, are methods, systems, and media useful for determining the health risk of a genomic sequence variant (GSV) in the nucleic acid sequence of an individual's genome. In certain embodiments, the DNA sequence comprises a sequence for an individual's whole genome. In certain embodiments, the DNA sequence comprises a sequence for only the high confidence regions of an individual's whole genome. In certain embodiments, the DNA sequence comprises a sequence for the high confidence region of an individual's whole genome as defined by the NA12878 Genome-In-A-Bottle call set (GiaB v2.19). In certain embodiments, the DNA sequence comprises a sequence for 90% of the high confidence region of an individual's whole genome as defined by the GiaB v2.19. In certain embodiments, the DNA sequence comprises a sequence for 80% of the high confidence region of an individual's whole genome as defined by the GiaB v2.19. In certain embodiments, the DNA sequence comprises a sequence for 70% of the high confidence region of an individual's whole genome as defined by the GiaB v2.19. In certain embodiments, the DNA sequence comprises a sequence of a plurality of contiguous nucleotides from an individual's genome. In certain embodiments, the DNA sequence comprises a sequence of at least 100 contiguous nucleotides from an individual's genome. In certain embodiments, the DNA sequence comprises a sequence of at least 1,000 contiguous nucleotides from an individual's genome. In certain embodiments, the DNA sequence comprises a sequence of at least 10,000 contiguous nucleotides from an individual's genome. In certain embodiments, the DNA sequence comprises a sequence of at least 100,000 contiguous nucleotides from an individual's genome. In certain embodiments, the DNA sequence comprises a sequence of at least 1,000,000 contiguous nucleotides from an individual's genome. In certain embodiments, the DNA sequence does not comprise the sequence of ribonucleic acid (RNA). In certain embodiments, the DNA sequence does not comprise the sequence of cDNA generated from ribonucleic acid (RNA).

### Genomic Health Risk

Described herein, are methods, systems, and media useful for determining the genomic health risk of a genomic sequence variant (GSV) in the DNA sequence of an individual's genome. Determining a genomic health risk encompasses several different or alternative steps. Further, the genomic health risk itself is with respect to an overall health risk or for specific diseases. In certain embodiments, determining the genomic health risk comprises determining a tolerability score for at least one GSV in an individual. In certain embodiments, determining the genomic health risk comprises determining an n-variant score for at least one GSV in an individual. In certain embodiments, determining the genomic health risk comprises determining a context dependent tolerance score for at least one region in which there is at least one GSV in an individual. In certain embodiments, determining the genomic health risk comprises determining a protein tolerability score for at least one GSV in an individual. In certain embodiments, the genomic health risk is determined using any single genomic health risk metric of this disclosure selected from the list consisting of: a tolerability score, an n-mer score, a context dependent tolerance score, and a protein tolerability score. In certain embodiments, the genomic health risk is determined using any two genomic health risk metrics of this disclosure selected from the list consisting of: a tolerability score, an n-mer score, a context dependent tolerance score, and a protein tolerability score. In certain embodiments, the genomic health risk is determined using any three genomic health risk metrics of this disclosure selected from the list consisting of: a tolerability score, an n-mer score, a context dependent tolerance score, and a protein tolerability score. In certain embodiments, the genomic health risk is determined using all of a tolerability score, an n-mer score, a context dependent tolerance score, and a protein tolerability score.

In certain embodiments, the genomic health risk is determined with respect to any single GSV of an individual. In certain embodiments, the genomic health risk is determined with respect to a plurality of GSVs of an individual. In certain embodiments, the genomic health risk is determined with respect to at least 10 GSVs of an individual. In certain embodiments, the genomic health risk is determined with respect to at least 100 GSVs of an individual. In certain embodiments, the genomic health risk is determined with respect to at least 1,000 GSVs of an individual. In certain embodiments, the genomic health risk is determined with respect to at least 10,000 GSVs of an individual. In certain embodiments, the genomic health risk is determined with respect to at least 100,000 GSVs of an individual.

In certain embodiments, the genomic health risk determined is an overall health risk defined as the increase or decrease in the likelihood of contracting any pathological condition. In certain embodiments, the genomic health risk is an arbitrary designation that communicates the increased risk of any given GSV. In certain embodiments, the genomic health risk is an arbitrary designation that communicates the increased risk of a plurality of GSVs. In certain embodiments, the genomic health risk is a percentage increase risk that any given GSV will be deleterious to the health of the individual. In certain embodiments, the genomic health risk is a percentage increase risk that a plurality of GSVs will be deleterious to the health of the individual. In certain embodiments, genomic health risk comprises the likelihood of contracting or being afflicted with diabetes, high blood pressure, cardiac arrhythmia, cardiovascular disease, atherosclerosis, stroke, non-alcoholic fatty liver disease, cirrhosis, dementia, bipolar disorder, depression, schizophrenia, anxiety disorder, autism, Asperger's syndrome, Parkinson's disease, Alzheimer's disease, Huntington's disease, cancer, breast cancer, prostate cancer, leukemia, melanoma, pancreatic cancer, colon cancer, stomach cancer, kidney cancer, liver cancer, an inborn error of metabolism, a genetically linked immunodeficiency, risk or protective alleles for the contraction. In certain embodiments, the genomic health risk is determined without GSVs known at the date of filing this disclosure that lead to a known disease, for example, known GSVs in the BRCA gene that lead to increased risk of breast cancer.

### Generation of Sequence Data

In certain embodiments, DNA sequence data for use with the methods, systems and media, described herein, is generated by any suitable method. In certain embodiments, the DNA sequence data is generated by Sanger sequencing. In certain embodiments, the DNA sequence data is generated by any next-generation sequencing technology. In certain embodiments, the DNA sequence data is generated, by way of non-limiting example, pyrosequencing, sequencing by synthesis, sequencing by ligation, ion semiconductor sequencing, or single molecule real time sequencing. In certain embodiments, the DNA sequence data is generated by any technology capable of generating 1 gigabase of nucleotide reads per 24 hour period. In certain embodiments, the DNA sequence data is obtained from a third party.

### Genomic Sequence Variants

In certain embodiments, GSVs for use with the methods, systems and media, described herein, are determined de novo during implementation of any of the methods. In certain embodiments, GSVs are determined by a third party and received by the party performing the method. In certain embodiments, determining a GSV encompasses receiving a list or file that comprises an individual's GSVs.

In certain embodiments, GSVs are determined by comparison with a reference genome. In certain embodiments, the reference genome is publicly available. In certain embodiments, the reference genome is NA12878 from the CEPH Utah reference collection. In certain embodiments, the reference genome is the GRCh38, Genome Reference Consortium human genome (build 38). In certain embodiments, the reference genome is any previous or subsequent build of the Genome Reference Consortium human genome. In certain embodiments, the reference genome is constructed from at least 1,000 human genomes. In certain embodiments, the reference genome is constructed from at least 10,000 human genomes. In certain embodiments, the reference genome is constructed from at least 100,000 human genomes. In certain embodiments, the reference genome is constructed from at least 1,000,000 human genomes. In certain embodiments, a GSV is a difference of a single nucleotide compared to a reference genome. In certain embodiments, a GSV is a difference of a plurality of contiguous nucleotides compared to a reference genome. In certain embodiments, a GSV is an insertion of one or more nucleotides compared to a reference genome. In certain embodiments, a GSV is a deletion of one or more nucleotides compared to a reference genome.

### Tolerability Score

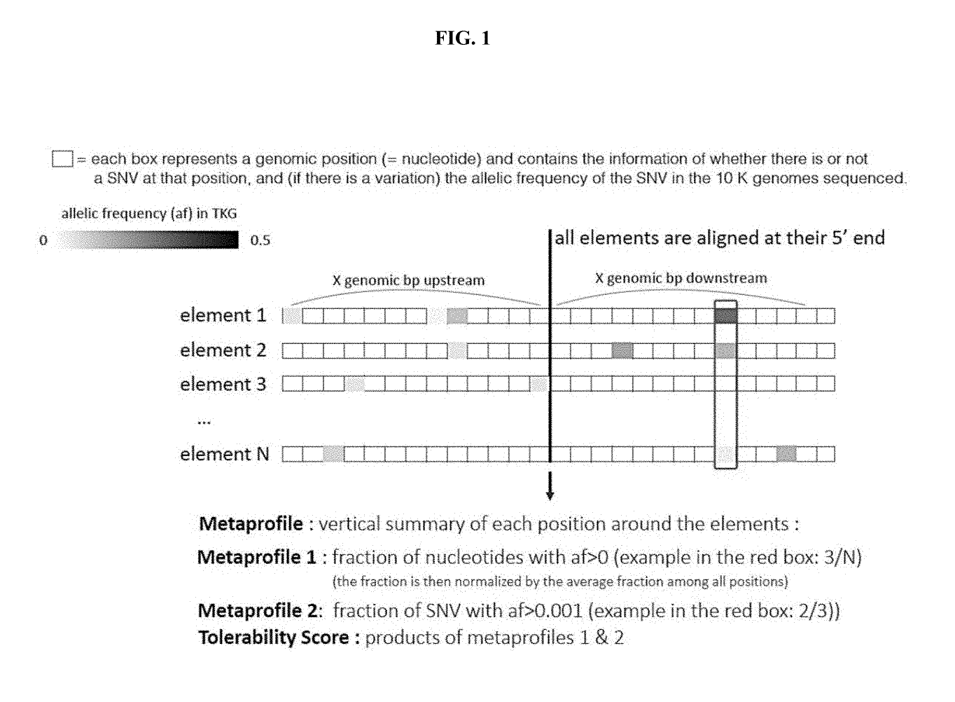

In certain embodiments, the methods, systems and media, described herein comprise determining a tolerability score for at least one GSV. In certain embodiments, the methods, systems and media, described herein comprise determining a tolerability score for a plurality of GSV. The concept of determining a tolerability score is captured in FIG. 1. A tolerability score is defined with regard to its position compared to a genetic landmark. In certain embodiments, the landmark is an arbitrary sequence or position in the genome. In certain embodiments, the landmark is a functional genetic element. In certain embodiments, the functional genetic element is a transcriptional start site, an initiation codon, an mRNA splice acceptor site, an mRNA splice donor site, a promoter element, an enhancer element, a regulatory element, a transcription factor binding site, a stop codon, a poly-adenylation site, a protein domain, a non-coding RNA or an exon-intron boundary. All landmarks that fall within a class of functional genetic elements in a plurality of genomes sequenced are then aligned at their 5 or 3 prime ends. The tendency of the genome to vary at a position x nucleotides from the land mark (the nucleotide variation score) is determined. In certain embodiments, a tolerability score is calculated from a minimum of 10 aligned genetic elements. In certain embodiments, a tolerability score is calculated from a minimum of 50 aligned genetic elements. In certain embodiments, a tolerability score is calculated from a minimum of 100 aligned genetic elements. In certain embodiments, a tolerability score is calculated from a minimum of 500 aligned genetic elements. In certain embodiments, a tolerability score is calculated from a minimum of 1,000 aligned genetic elements. In certain embodiments, a tolerability score is calculated from a minimum of 5,000 aligned genetic elements. In certain embodiments, a tolerability score is calculated from a minimum of 10,000 aligned genetic elements.

The nucleotide variation score in the plurality of genomes is determined for a position x bases upstream or downstream of the above mentioned landmark. In certain embodiments, the position is less than 10, 20, 30, 40, 50, 60, 70, 80, 90, 100, 200, 300, 400, 500, 600, 700, 800, 900, or 1,000 bases, including increments therein, upstream or downstream from the landmark. The nucleotide variation score is then normalized to the average variability for all positions within x nucleotides of the landmark or genetic element. In certain embodiments, this normalization occurs in 100 to 1500 base pairs. The nucleotide variation score is then multiplied by the fraction of all alleles at that position x bases from the landmark that exceed 0.0001 (the allele proportion score, where the maximal allelic proportion is 0.5 in a population). In certain embodiments, the tolerability score is a function of the nucleotide variation score and the fraction of all alleles at that position x bases from the landmark that exceed 0.0001.This yields the tolerability score for a position x bases from a given landmark. In certain embodiments, the allele proportion score is determined as the fraction of all alleles at a position x bases from the landmark that exceeds 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, or 0.010. If an individual possesses a GSV x bases from a landmark the tolerability sore for that position is then correlated with the GSV.

In certain embodiments, a tolerability score that is below 0.01 indicates an increase in the genomic health risk for a given GSV. In certain embodiments, a tolerability score that is below 0.02 indicates an increase in the genomic health risk for a given GSV. In certain embodiments, a tolerability score that is below 0.03 indicates an increase in the genomic health risk for a given GSV. In certain embodiments, a tolerability score that is below 0.04 indicates an increase in the genomic health risk for a given GSV. In certain embodiments, a tolerability score that is below 0.05 indicates an increase in the genomic health risk for a given GSV. In certain embodiments, a tolerability score that is below 0.06 indicates an increase in the genomic health risk for a given GSV. In certain embodiments, a tolerability score that is below 0.07 indicates an increase in the genomic health risk for a given GSV. In certain embodiments, a tolerability score that is below 0.08 indicates an increase in the genomic health risk for a given GSV. In certain embodiments, a tolerability score that is below 0.09 indicates an increase in the genomic health risk for a given GSV. In certain embodiments, a tolerability score that is below 0.10 indicates an increase in the genomic health risk for a given GSV. In certain embodiments, a tolerability score that is below 1 indicates an increase in the genomic health risk for a given GSV. In certain embodiments, a tolerability score that is below 0.12 indicates an increase in the genomic health risk for a given GSV. In certain embodiments, a tolerability score that is below 0.13 indicates an increase in the genomic health risk for a given GSV. In certain embodiments, the genomic health risk is increased by at least 20%. In certain embodiments, the genomic health risk is increased by at least 50%. In certain embodiments, the genomic health risk is increased by at least 100%. In certain embodiments, the genomic health risk is increased by at least 200%. In certain embodiments, the genomic health risk is increased by at least 300%. In certain embodiments, the genomic health risk is increased by at least 400%. In certain embodiments, the genomic health risk is increased by at least 500%. In certain embodiments, the genomic health risk is increased by at least 1000%.

### Tolerability Score Examples

Position 117587738 on chromosome 7 has a tolerance score of 0.0159 and a variation at that position has been associated with Cystic fibrosis (ClinVar entry: NM_000492.3(CFTR):c.1585-1G>A AND Cystic fibrosis).

Position 32326240 on chromosome 13 has a tolerance score of 0.0137 and a variation at that position has been associated with Breast ovarian cancer (ClinVar entry: NM_000059.3(BRCA2):c.476-2A>G AND Breast-ovarian cancer, familial 2).

Position 47480818 on chromosome 2 has a tolerance score of 0.0258 and a variation at that position has been associated with Lynch syndrome (ClinVar entry: NM_000251.2(MSH2):c.2581C>T (p.G1n861Ter) AND Lynch syndrome).

n-Variant Score

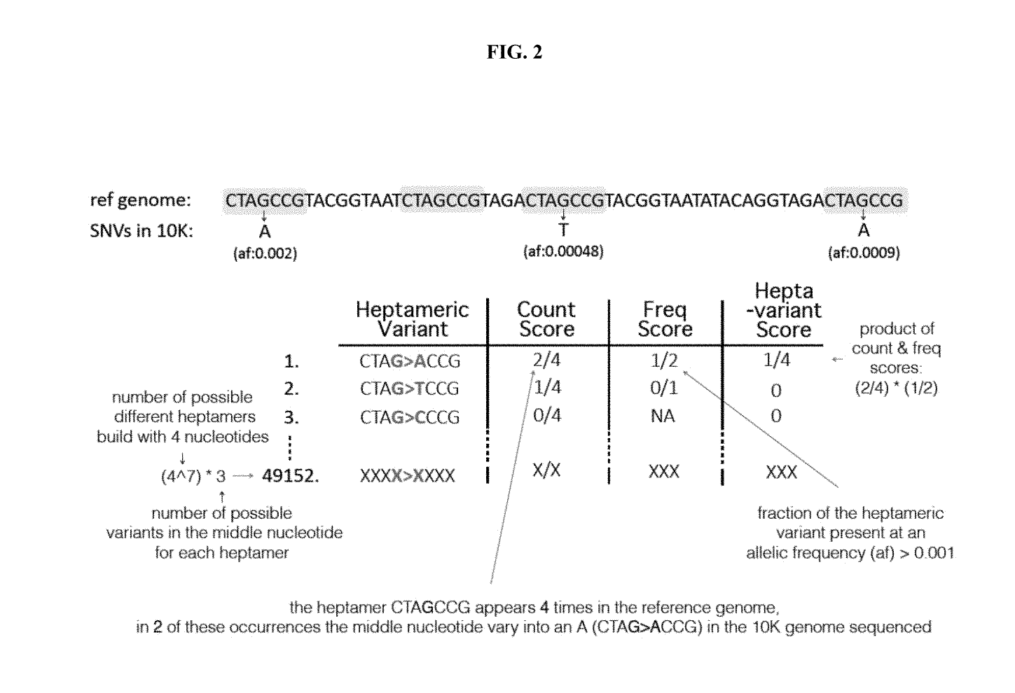

In certain embodiments, the methods, systems and media, described herein comprise determining an n-variant score for at least one GSV. In certain embodiments, the methods, systems and media, described herein comprise determining an n-variant score for a plurality of GSV. The concept of determining an n-variant score, in this case n=7, is captured in FIG. 2. Given 4 different nucleotides there are 4⁷ (16,384) different 7-mers (heptamers) possible. Every GSV will be situated, in this case, in the middle, of at least one of these 16,384 different heptamers, thus each GSV will create a heptameric variant from an existing heptamer. Since the variation at that GSV could theoretically be any of three different bases, the total variant heptamers possible are 16,384×3=49,152. Unexpectedly, not all variant heptamers are equally possible. First, a count score is determined, the count score comprises the number of instances a certain heptamer variant occurs in a plurality of genomes sequenced divided by the number of instances the non-mutated heptamer appears in the reference genome. This count score is then multiplied by the proportion of the specific GSV that gave rise to the variant heptamer that were present at an allelic frequency of more than 1 in a 1000. Since every nucleotide is a part of an n-mer, an n-variant score can be calculated for each nucleotide in a haploid genome. In certain embodiments, n can be any number. In certain embodiments, n is equal to 3, 4, 5, 6, 7, 8, 9, 10, 11, 12, 13, 14, 15, 16, 17, 18, 19, or 20. In certain embodiments, the GSV occurs in the center of the n-mer. In certain embodiments, the GSV occurs at a position that is not the center of the n-mer. In certain embodiments, the GSV occurs at the 5 prime end of the n-mer. In certain embodiments, the GSV occurs at the three prime end of the n-mer.

In certain embodiments, an n-variant score that is below 0.001 indicates an increase in the genomic health risk for a given GSV. In certain embodiments, an n-variant score that is below 0.002 indicates an increase in the genomic health risk for a given GSV. In certain embodiments, an n-variant score that is below 0.003 indicates an increase in the genomic health risk for a given GSV. In certain embodiments, an n-variant score that is below 0.004 indicates an increase in the genomic health risk for a given GSV. In certain embodiments, an n-variant score that is below 0.005 indicates an increase in the genomic health risk for a given GSV. In certain embodiments, an n-variant score that is below 0.006 indicates an increase in the genomic health risk for a given GSV. In certain embodiments, an n-variant score that is below 0.007 indicates an increase in the genomic health risk for a given GSV. In certain embodiments, an n-variant score that is below 0.08 indicates an increase in the genomic health risk for a given GSV. In certain embodiments, n-variant score that is below 0.009 indicates an increase in the genomic health risk for a given GSV. In certain embodiments, n-variant score that is below 0.010 indicates an increase in the genomic health risk for a given GSV. In certain embodiments, n-variant score that is below 0.011 indicates an increase in the genomic health risk for a given GSV. In certain embodiments, n-variant score that is below 0.012 indicates an increase in the genomic health risk for a given GSV. In certain embodiments, n-variant score that is below 0.013 indicates an increase in the genomic health risk for a given GSV. In certain embodiments, the genomic health risk is increased by at least 20%. In certain embodiments, the genomic health risk is increased by at least 50%. In certain embodiments, the genomic health risk is increased by at least 100%. In certain embodiments, the genomic health risk is increased by at least 200%. In certain embodiments, the genomic health risk is increased by at least 300%. In certain embodiments, the genomic health risk is increased by at least 400%. In certain embodiments, the genomic health risk is increased by at least 500%. In certain embodiments, the genomic health risk is increased by at least 1000%. In certain embodiments, the n-variant score allows the identification of pathogenic variants (health risk associated) without the need for annotation.

n-Variant Score Examples

Position 43115730 on chromosome 17 has an heptamer tolerability score of 0.000397 for the variant T>A and this variant has been associated with Breast ovarian cancer (ClinVar entry: NM_007294.3(BRCA1):c.130T>A (p.Cys44Ser) AND Breast-ovarian cancer, familial 1).

Position 37028836 on chromosome 3 has an heptamer tolerability score of 0.000393 for the variant A>T and this variant has been associated with Lynch syndrome (ClinVar entry: NM_000249.3(MLH1):c.1462A>T (p.Lys488Ter) AND Lynch syndrome).

Position 108335959 on chromosome 11 has an heptamer tolerability score of 0.000388 for the variant A>T and this variant has been associated with Hereditary cancer-predisposing syndrome (ClinVar entry: NM_000051.3(ATM):c.8266A>T (p.Lys2756Ter) AND Hereditary cancer-predisposing syndrome).

### Context Dependent Tolerance Score

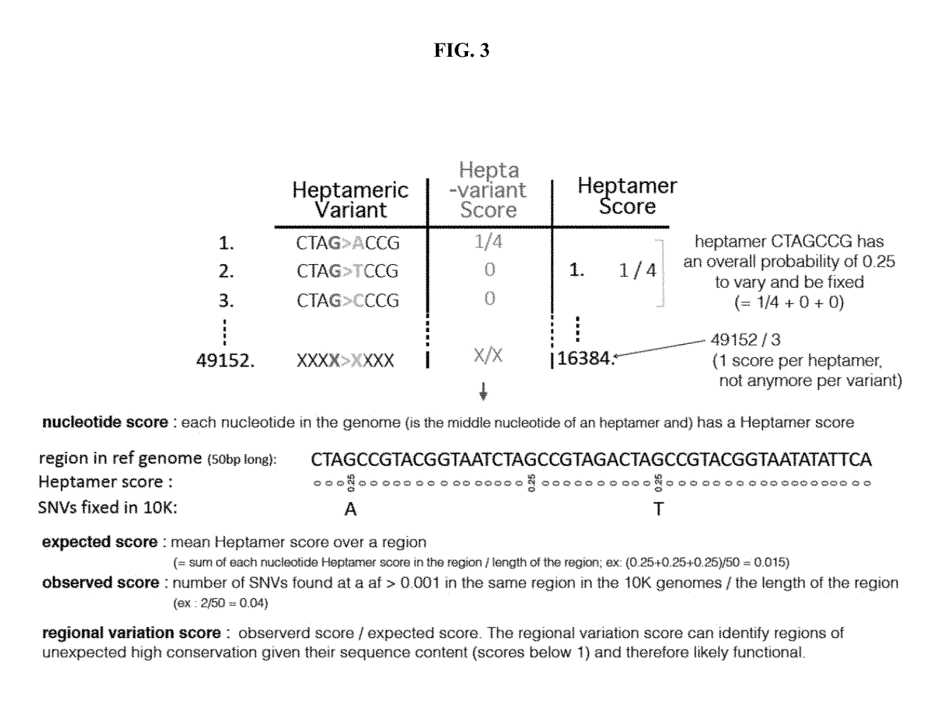

In certain embodiments, the methods, systems and media, described herein comprise determining a context dependent tolerance score (regional variation score) for the region in which at least one GSV occurs. In certain embodiments, the methods, systems and media, described herein comprise determining a context dependent tolerance score for the region in which at least one GSV occurs. As noted previously an n-variant score can be determined for each nucleotide in the genome. In FIG. 3, the context dependent tolerance score is determined as an expected variation in a region of the genome versus the observed variation for that genome. Any given n-mer will have an overall probability to vary. In the case of a heptamer, there are 16,384 different possible heptamers. A variant at a given position in the heptamer will vary at a given frequency in a reference genome this is the global probability to vary. This global probability to vary is summed over the entire length of the region and divided by the length of the region, measured in nucleotides, giving the expected context dependent tolerance score. This number is then compared to the observed context dependent tolerance score, which is given by the number of single nucleotide variations in the plurality of genomes divide by the length of the region measured in nucleotides. The lower the context dependent tolerance (observed variation lower than expected variation) score the less tolerant the region is to variation and the greater the likelihood that a GSV located in this region will be deleterious. One of skill in the art will appreciate that the context dependent tolerance score is a function of the expected context dependent tolerance score and the observed context dependent tolerability score. By way of non-limiting example, the observed context dependent tolerance score may be divided by the expected context dependent tolerance score; the expected context dependent tolerance score may be subtracted from the observed context dependent tolerance score, the observed context dependent tolerance score may be subtracted from the expected context dependent tolerance score; the observed context dependent tolerance score may be added to the expected context dependent tolerance score.

In certain embodiments, the region for which the global probability to vary is between 10 and 10,000 nucleotides in length. In certain embodiments, the region is between 10 and 1,000 nucleotides in length. In certain embodiments, the region is between 10 and 500 nucleotides in length. In certain embodiments, the region is between 10 and 100 nucleotides in length. In certain embodiments, the region is between 100 and 200 nucleotides in length. In certain embodiments, the region is between 120 and 180 nucleotides in length. In certain embodiments, the region is between 140 and 160 nucleotides in length. In certain embodiments, the region is between 300 and 700 nucleotides in length. In certain embodiments, the region is between 400 and 600 nucleotides in length. The region can be any length that is able to be practically analyzed using computer aided means including lengths in excess of 1,000; 5,000; 10,000; 50,000; or 100,000 nucleotides.

In certain exemplary embodiments, if the context dependent tolerance score is represented as an observed context dependent tolerance score divided by the expected context dependent tolerance score a context dependent tolerance score below 1 increases the genomic health risk of a given GSV. In certain embodiments, a GSV that occurs in a region with a context dependent tolerance score below 0.9 increases the genomic health risk of a given GSV. In certain embodiments, a GSV that occurs in a region with a context dependent tolerance score below 0.8 increases the genomic health risk of a given GSV. In certain embodiments, a GSV that occurs in a region with a context dependent tolerance score below 0.7 increases the genomic health risk of a given GSV. In certain embodiments, a GSV that occurs in a region with a context dependent tolerance score below 0.6 increases the genomic health risk of a given GSV. In certain embodiments, a GSV that occurs in a region with a context dependent tolerance score below 0.5 increases the genomic health risk of a given GSV. In certain embodiments, a GSV that occurs in a region with a context dependent tolerance score below 0.4 increases the genomic health risk of a given GSV. In certain embodiments, a GSV that occurs in a region with a context dependent tolerance score below 0.3 increases the genomic health risk of a given GSV. In certain embodiments, a GSV that occurs in a region with a context dependent tolerance score below 0.2 increases the genomic health risk of a given GSV. In certain embodiments, a GSV that occurs in a region with a context dependent tolerance score below 0.1 increases the genomic health risk of a given GSV. In certain embodiments, the genomic health risk is increased by at least 20%. In certain embodiments, the genomic health risk is increased by at least 50%. In certain embodiments, the genomic health risk is increased by at least 100%. In certain embodiments, the genomic health risk is increased by at least 200%. In certain embodiments, the genomic health risk is increased by at least 300%. In certain embodiments, the genomic health risk is increased by at least 400%. In certain embodiments, the genomic health risk is increased by at least 500%. In certain embodiments, the genomic health risk is increased by at least 1000%.

The context dependent tolerance score is able to identify potentially pathogenic genomic sequence variants without any a priori knowledge about the genomic location of the sequence variant. In certain embodiments, the context dependent variation score allows the identification of pathogenic (health risk associated) variants without the need for annotation. In certain embodiments, the context dependent variation score allows the identification of pathogenic (health risk associated) variants without the need for functional annotation.

In certain embodiments, the genomic health risk of a particular variant is defined as pathogenic if it falls in a region of the genome in the top 10% of conserved regions. In certain embodiments, the genomic health risk of a particular variant is defined as pathogenic if it falls in a region of the genome in the top 5% of conserved regions. In certain embodiments, the genomic health risk of a particular variant is defined as pathogenic if it falls in a region of the genome in the top 2% of conserved regions. In certain embodiments, the genomic health risk of a particular variant is defined as pathogenic if it falls in a region of the genome in the top 1% of conserved regions.

In certain embodiments, the genomic health risk of a particular variant is defined as pathogenic if it in the top 10% of conserved genomic loci. In certain embodiments, the genomic health risk of a particular variant is defined as pathogenic if it falls in a region of the genome in the top 5% of genomic loci. In certain embodiments, the genomic health risk of a particular variant is defined as pathogenic if it falls in a region of the genome in the top 2% of genomic loci. In certain embodiments, the genomic health risk of a particular variant is defined as pathogenic if it falls in a region of the genome in the top 1% of genomic loci.

### Context Dependent Variation Score Examples

In these examples, the expected context dependent tolerance score (CDTS) is subtracted from the observed context dependent tolerance score to yield the context dependent tolerability score. In this case the more negative the score the more potentially pathogenic the variant. In general, when the CDTS is a subtraction function, a number less than zero indicates an increased health risk of a given variant. In certain embodiments, a CDTS of less than 0, −1, −2, −3, −4, −5, −6, −7, −8, −9, −10, −11, or −12 indicates an increased health risk.

ClinVar pathogenic variant (entry NM_000249.3(MLH1):c.2T>A (p.Met1Lys) AND Lynch syndrome), position 36993549 on chromosome 3 is associated with Lynch syndrome and has a context dependent tolerance score of −12.0987.

ClinVar pathogenic variant (entry NM_000492.3(CFTR):c.350G>A (p.Arg117His) AND Cystic fibrosis), position 117530975 on chromosome 7 is associated with Cystic fibrosis and has a context dependent tolerance score of −4.16129

ClinVar pathogenic variant (entry NM_006516.2(SLC2A1):c.377G>A (p.Arg126His) AND Glucose transporter type 1 deficiency syndrome), position 42930765 on chromosome 1 is associated with Glucose transporter type 1 deficiency syndrome and has a context dependent tolerance score of −9.09988.

### Protein Tolerability Score

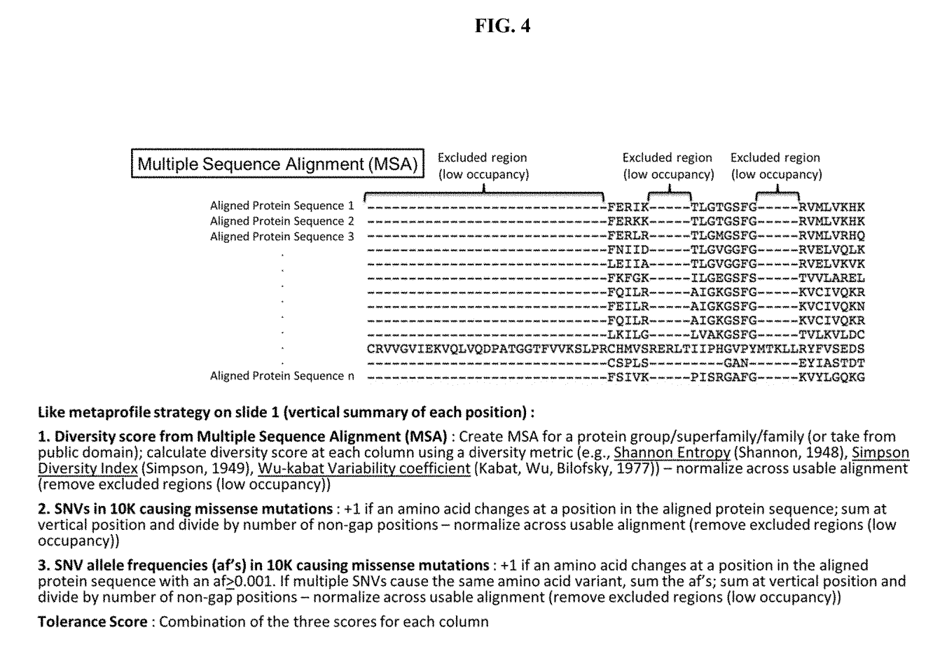

In certain embodiments, the methods, systems and media, described herein comprise determining a protein tolerability score for at least one GSV. In certain embodiments, the methods, systems and media, described herein comprise determining a protein tolerability score for a plurality of GSV. The concept of determining a protein tolerability score is captured in FIG. 4. The protein tolerability score is analogous to the tolerability score except that it accounts for conservation among proteins and not necessarily nucleotides. For the protein tolerability score a multiple sequence alignment is used to align proteins from a certain class or family. A diversity score is assigned to each vertically aligned amino acid column. In certain embodiments, the diversity score is calculated using the Shannon-Entropy, Simpson diversity index, WU-Kabat score, or any other amino acid diversity scoring algorithm. A missense score is determined. The missense score is determined by the variance observed in a plurality of genomes at the corresponding position, which leads to an amino acid mutation. Finally, a protein allele frequency score is determined. In certain embodiments, the protein tolerability score is the arithmetic product of the diversity score, the missense score and the protein allele frequency score. In certain embodiments, the protein tolerability score is an average of the diversity score, the missense score and the protein allele frequency score. In certain embodiments, the protein tolerability score is a weighted average of the diversity score, the missense score and the protein allele frequency score.

In certain embodiments, the protein family is any family of proteins that exhibit an evolutionary relationship, such as kinases. In certain embodiments, the protein family is any family of proteins that exhibit an evolutionary relationship and possess at least 95% similarity. In certain embodiments, the protein family is any family of proteins that exhibit an evolutionary relationship and possess at least 90% similarity. In certain embodiments, the protein family is any family of proteins that exhibit an evolutionary relationship and possess at least 85% similarity. In certain embodiments, the protein family is any family of proteins that exhibit an evolutionary relationship and possess at least 80% similarity. In certain embodiments, the protein family is any family of proteins that exhibit an evolutionary relationship and possess at least 75% similarity. In certain embodiments, the protein family is any family of proteins that exhibit an evolutionary relationship and possess at least 70% similarity. In certain embodiments, a protein tolerability score that is below 0.1 indicates an increase in the genomic health risk for a given GSV. In certain embodiments, a protein tolerability score that is below 0.05 indicates an increase in the genomic health risk for a given GSV. In certain embodiments, a protein tolerability score that is below 0.01 indicates an increase in the genomic health risk for a given GSV. In certain embodiments, a protein tolerability score that is below 0.005 indicates an increase in the genomic health risk for a given GSV.

### Functional Genomic Application for Tolerability and Variation Metrics

There is an established relationship between functional units and sequence conservation. Regions that are both functional and conserved are deemed essential for biology. Disclosed herein, are methods of using the regional score to enable the identification, and targeting for analysis and sequencing, of those parts of the human genome that are most functionally relevant, and, thus, most relevant for health.

The functional genome comprises regions that are known to have a biological role and share properties that assimilate them to probable functional units, despite being poorly annotated.

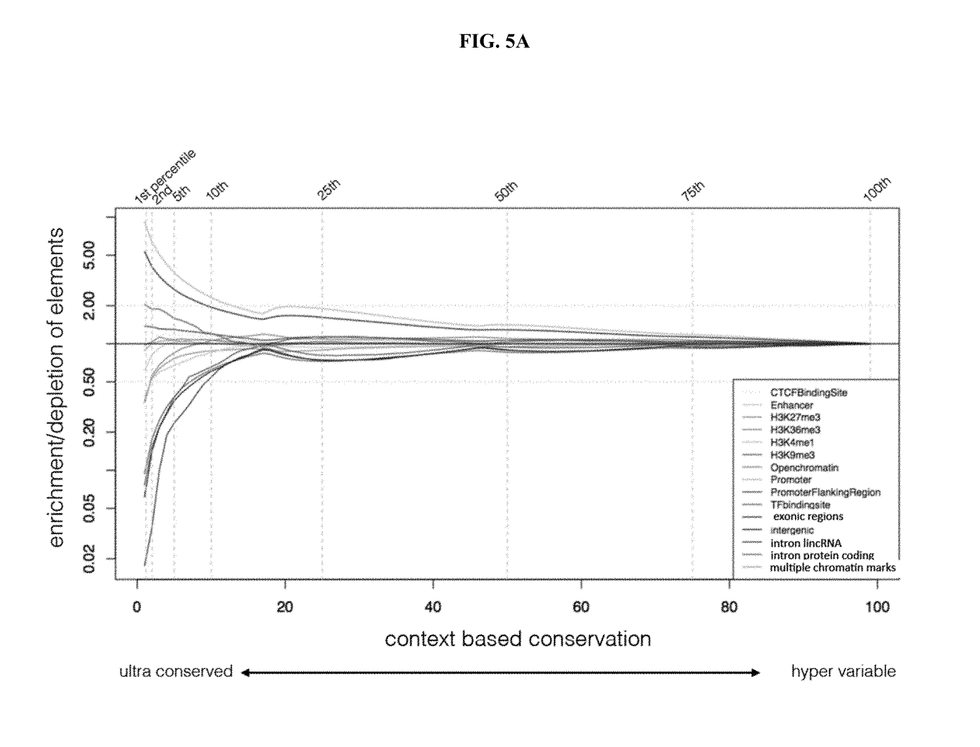

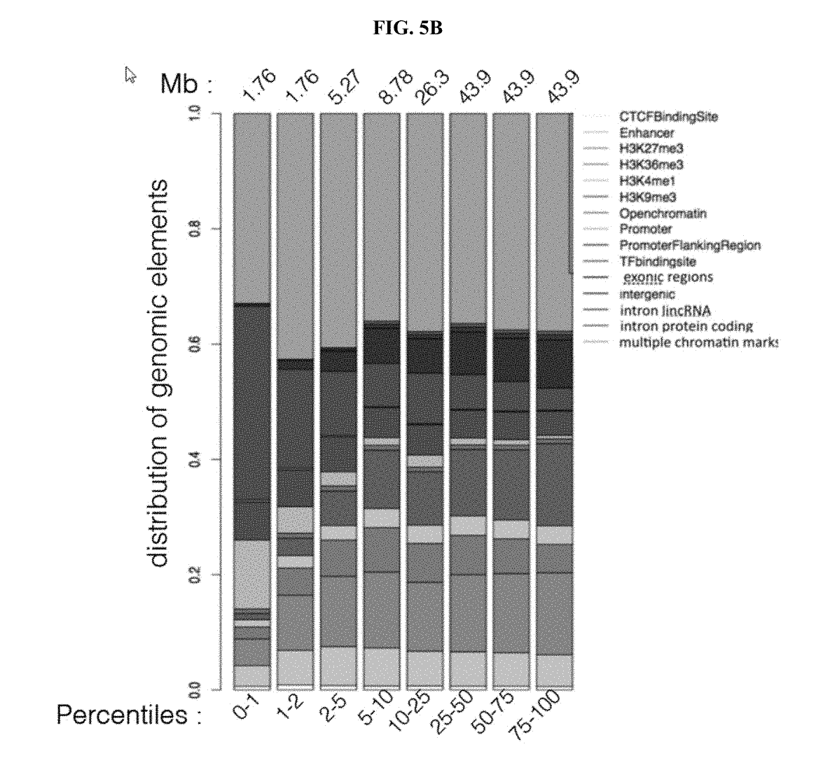

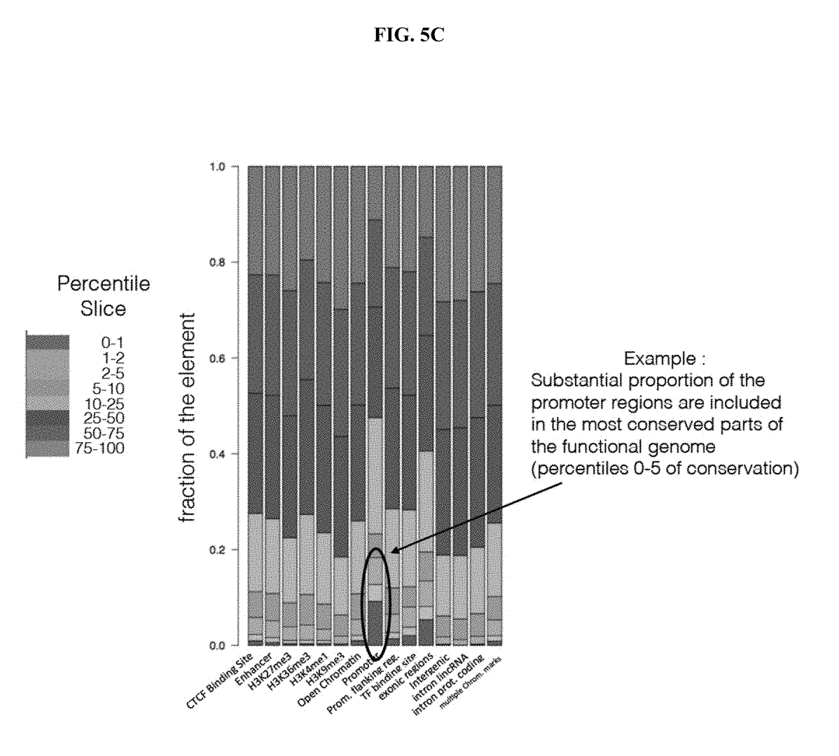

Referring to FIG. 5A, presented is the pattern of enrichment and depletion of genomic elements in regions with marked context-based conservation (lowest regional score). Specifically, in the 1^{st} percentile of regional scores (most conserved) we observe an enrichment of up to 10-fold in promoter sequences, and 5-fold in exonic sequences. In parallel, at the 1st percentile of regional score, there is up to 10 to 50-fold depletion in intronic and intergenic sequences.

Referring to FIG. 5B, the analysis of pattern of enrichment allowed the detailed inspection of the genomic content for different levels of regional scores. For all genome elements, there are subsets of context-based conserved elements (lower range of regional score). For example, in the 1.76 Mb of sequence in the 1^{st} percentile 0.6 Mb of sequence represents conserved exonic sequences, and over 1.1 Mb contain other important genomics elements. Discovery is facilitated—as illustrated by the identification of 8 Kb of intergenic region with features of profound context-based conservation.

Referring to FIG. 5C, the most context-based conserved region is of particular interest for targeted analysis and detailed annotation. FIG. 5C highlights the proportion of each genomic element that can be classified as functionally constrained at different percentiles of context-based conservation. For example, the 5^{th} percentile contains 18% of the promoters, 13% of the exonic regions, and decreasing proportions of other genomic elements.

Referring to FIGS. 5A-5C, any of the methods of this disclosure can be used in a method to identify functional genomic regions of the genome. These regions can be prioritized for sequence analysis or targeted sequencing. In certain embodiments any one or more of a tolerability score, an n-variant score, a context dependent tolerance score, and a protein tolerability score can be used prioritize a part of the genome using a functional genomic approach.

The methods of this disclosure can be used to develop a functional genomic assay. This functional genomic assay can integrate any of the methods described herein, including a context dependent tolerance score. The functional genomic assay comprises a step of obtaining a nucleic acid sequence from a biological sample from an individual; and determining a presence of at least one genomic sequence variant in a region that is highly conserved; wherein the region that is highly conserved is a region wherein an observed context dependent tolerance score is greater than an expected context dependent tolerance score, wherein the expected context dependent tolerance score is the overall probability to vary of a unique sequence of n-nucleotides in length in a certain region of x nucleotides in length in a plurality of genomes, and the observed context dependent tolerance score is a number of genomic sequence variants in a certain region of x nucleotides in length actually observed and fixed in the plurality of genomes as a function of a length of the region. In a certain instance, the at least one genomic sequence variant is in a non-coding region.

Suitable biological samples can comprise oral swabs, whole-blood samples, peripheral blood mononuclear cells obtained from whole blood, plasma samples, serum samples, biopsy samples (both normal and malignant tissue), semen samples, fecal/stool samples. Nucleic acids can be isolated in these samples using methods well known in the art and appropriate nucleotides for determining genomic sequence variants, can comprise RNA, mRNA, genomic DNA (including circulating cell-free DNA derived from nuclear DNA). In certain instances, the DNA does not comprise mitochondrial DNA or DNA derived from sex-chromosomes.

The step of the determining a presence of at least one genomic sequence variant in a region that is highly conserved can be greatly expanded. In some cases. greater than 10, 20, 30, 40, 50, 60, 70, 80, 90, 100, 200, 300, 400, 500, 600, 700, 800, 900, or 1,000 genomic sequence variants can be determined in greater than 2, 3, 4, 5, 6, 7, 8, 9, 10, 20, 30, 40, 50, 60, 70, 80, 90, 100, 200, 300, 400, 500, 600, 700, 800, 900, or 1000 highly conserved regions. In some cases genomic sequence variants can be determined in greater than 10,000; 20,000; 30,000; 40,000; 50,000; 60,000,; 70,000; 80,000; 90,000 or 100,000 highly conserved regions. In some cases genomic sequence variants can be determined in the most highly conserved 0.1%, 1%, 2%, 3%, 4%, 5%, 6%, 7%, 8%, 9%, 10% regions of the genome as determined by the method herein or the context dependent tolerability score. A list of exemplar highly conserved regions corresponding to the most conserved 1% of genomic regions is shown in Table 5 (49523-703-201-TABLES.txt) submitted in text format with the instant application. Listed is the human chromosome number and the range of coordinates from X to X (e.g., chr1 902440 903230). Coordinates given are with regard to the Genome Reference Consortium GRCh38 build. Any one or more of these genomic regions are considered highly conserved for the purposes of functional genomic assay detailed herein.

The sequences can be determined using any method known inn the art that is sufficiently high throughput to enrich and identify a plurality of genomic sequence variants, such as, for example, next-generation sequencing (e.g., sequencing by synthesis, ion-semiconductor sequencing, or single molecule real-time sequencing) nucleotide array, massively-multiplex PCR, molecular inversion probes, padlock probes, or connector inversion probes. In certain instances the step of obtaining a nucleic acid sequence from a biological sample comprises receiving nucleotide sequence data from a third-party including commercial third parties such as 23andme. Additionally, the sequences may be received as raw data or as pre-called variants in a variant call format (.vcf) file. In certain instances greater than 10; 100; 1,000; 10,000; 100,000; 1,000,000; 2,000,000; or 3,000,000 GSVs, including increments therein, can be determined.

The genomic sequence variants (GSVs) determined include both germline and somatic mutations. For example, determining somatic GSVs from a biopsy sample, when compared to a normal germline control sample, can help to identify regions that are causative and contribute to an individual's malignancy allowing for rational selection of a treatment option. This treatment option can comprise specific drugs that target specific pathways or modalities that are associated with particular genomic mutations. The advantage of this functional genomic assay is that no previous knowledge concerning the potential pathogenicity of a particular locus is needed. The genomic sequence variant can include SNPS, indels, translocations, repetitions, or copy number variations.

The pathogenicity of a GSV can be determined with respect to a candidate or known disease associated gene. In certain aspect the GSV can be within 2 megabases, 1 megabase, 1 kilobase, 200 base pairs, or 100 base pairs of a genomic feature of a known disease associated gene, such as a spice acceptor site, splice donor site, transcriptional start site, or promoter or enhance region.

Additional advantages of the functional genomic assay are that it is amenable to simultaneous analysis of GSVs without any pre-annotation. In certain instances greater than 10; 100; 1,000; 10,000; 100,000; 1,000,000; 2,000,000; or 3,000,000, including increments therein, can be analyzed without any appreciable additional cost from computing sources used.

For the described functional genomic assay, the unique sequence of n-nucleotides in length can be any number larger than 2 and smaller than 20. In certain embodiments, n is equal to 3, 4, 5, 6, 7, 8, 9, 10, 11, 12, 13, 14, 15, 16, 17, 18, 19, or 20.

For the described functional genomic assay, the certain region of x nucleotides in length can be greater than 10, 20, 20, 100, 200, 300, 400, 500, 600, 700, 800, 900, or 1,000 base pairs, including increments therein. The certain region of x nucleotides in length can be less than, 20, 20, 100, 200, 300, 400, 500, 600, 700, 800, 900, or 1,000 base pairs, including increments therein. In certain embodiments, the certain region of x nucleotides in length can be between 10 and 10,000 nucleotides in length; between 10 and 1,000 nucleotides in length; between 10 and 500 nucleotides in length; between 10 and 100 nucleotides in length; between 100 and 200 nucleotides in length; between 120 and 180 nucleotides in length; between 140 and 160; between 300 and 700; and between 400 and 600 nucleotides in length. The region can be any length that is able to be practically analyzed using computer aided means including lengths in excess of 1,000; 5,000; 10,000; 50,000; or 100,000 nucleotides, including increments therein.

The probability to vary is calculated from a plurality of genomes in some instance the plurality of genomes is greater than 10,000, 20,000; 30,000; 40,000; 50,000; 60,000; 70,000; 80,000; 90,000; 100,000; 200,000, 300,000; 400,000; 500,000; 600,000; 700,000; 800,000; 900,000; or 1,000,000 individual genomes, including increments therein. The probability to vary can be calculated from the allele frequency of all known alleles located in a certain region of x nucleotides in length, and optionally normalized to the length of the certain region of x nucleotides in length.

In certain instances, the functional genomic assay comprises determining the presence of genomic sequence variant of any 1, 2, 3, 4, 5, 6, 7, 8, 9, 10, 11, 20, 30, 40, 50, 60, 70, 80, 90, 100, 200, 300, 400, 500, 600, 700, 800, 900 or more variants, including increments therein, in an individual given in Table 1. In certain instances, the functional genomic assay comprises determining the presence of genomic sequence variant of all variants given in Table 1. In certain instances, the functional genomic assay comprises determining the presence of a genomic sequence variant of any 1, 2, 3, 4, 5, 6, 7, 8, 9, 10, 11, 20, 30, 40, 50, 60, 70, 80, 90, 100, 200 or more variants, including increments therein, in an individual given in Table 2. In certain instances, the functional genomic assay comprises determining the presence of genomic sequence variant of all variants given in Table 2. In certain instances, the functional genomic assay comprises determining the presence of genomic sequence variant of any 1, 2, 3, 4, 5, 6, 7, 8, 9, 10, 11, 20, 30, 40, 50, 60, 70, 80, 90, 100, 110 or more variants, including increments therein, in an individual given in Table 3. In certain instances, the functional genomic assay comprises determining the presence of genomic sequence variant of all variants given in Table 3. In certain instances, the functional genomic assay comprises determining the presence of genomic sequence variant of any of 1, 2, 3, 4, 5, 6, 7, 8, 9, 10, 11, 20, 30, 40 or more variants, including increments therein, in an individual given in Table 4. In certain instances, the functional genomic assay comprises determining the presence of genomic sequence variant of any of 1, 2, 3, 4, 5, 6, 7, 8, 9, 10, 11, 20, 30, 40 or more variants, including increments therein, in an individual given in Table 4.

The functional genomic assay described is useful for determining a likelihood of a subsymptomatic disease, such as, a cancer, a metabolic disorder, a physiological disorder, or an autoimmune or inflammatory disorder. In addition, the assay is useful as a predictive measure to determine likelihood of developing a disease, such as, a cancer, a metabolic disorder, a physiological disorder, or an autoimmune or inflammatory disorder. This functional genomic assay can be used as a prognostic indicator for treatment and be performed multiple times on the same individual to guide treatment. These methods can be applied to a biopsy or a cell-free nucleic acid isolated from the plasma, for example, determine a prognosis of a cancer or to determine the malignant potential of a biopsy. In a certain aspect, the cell-free nucleic acid is an mRNA or DNA. The DNA can be derived from a linear chromosome in the nucleus of a cell and in certain aspects is not derived from mitochondria or a sex-chromosome. The functional genomic assay can assign a certain GSV as high risk when the observed context dependent tolerance score is 5%, 10%, 20%, 25%, 30%, 35%, 40%, 45%, 50%, 60%, 70%, 80%, 90%, 100%, 150%, or 200%, including increments therein, greater than an expected context dependent tolerance score for that GSV. In addition the functional genomic assay can determine a risk for a plurality of GSVs in some cases greater than 3, 4, 5, 6, 7, 8, 9, 10, 20, 30, 40, 50, 60, 70, 80, 90, 100, 200, 300, 400, 500, 600, 700, 800, 900, or 1000, including increments therein. The risk can be averaged or summed for the specific GSVs. The GSV can be in a certain part of the genome within 100 bp, 500 bp, 1 kb, 5 kb, or 10 kb, including increments therein, of a functional motif such as a splice acceptor site, splice donor site, transcriptional start site, a promoter, or enhancer element. In certain cases these, functional motifs are associated with a gene known to play a role in cancer, such as, a rector tyrosine kinase (e.g., epidermal growth factor receptor (EGFR), platelet-derived growth factor receptor (PDGFR), and vascular endothelial growth factor receptor (VEGFR), HER2/neu, ROR1); cytoplasmic tyrosine kinases (e.g., Src-family, Syk-ZAP-70 family, and BTK family of tyrosine kinases, BCR/ABL); cytoplasmic Serine/threonine kinases and their regulatory subunits (e.g., Raf kinase, and cyclin-dependent kinases); a regulatory GTPase (e.g., a Ras gene); a transcription factor (e.g., myc), or a tumor suppressor gene (e.g., p53, BRCA1, BRCA2, RB, PTEN, or pVHL, APC, CD95, ST5, YPEL3, ST7, and ST14).

### Data Structures

In certain embodiments, any of a tolerability score, an n-variant score, a context dependent tolerance score, and a protein tolerability score can be pre-determined. In certain embodiments, a health care professional compares any one or more GSVs to a list, a spreadsheet or file with pre-determined health metrics. In certain embodiments, any of the health metrics are pre-determined for each nucleotide in the genome and accessible through a software program, on-line service or portal.

### Systems

In certain embodiments, described herein, are systems to identify the relative genomic health risk of a genomic sequence variant of an individual comprising: a DNA sequence for the individual; a system to determine at least one genomic sequence variant in the DNA sequence of the individual; wherein the genomic sequence variant is a difference of at least one nucleotide in the individual when compared to a corresponding position in a reference genome; and a system to compare the at least one genomic sequence variant of the individual to a tolerability score at a corresponding position within x-nucleotides of a genetic element, wherein the tolerability score comprises a function of a nucleotide variation score and an allele proportion score, wherein the nucleotide variation score is the variance observed in a plurality of genomes at the corresponding position, and the allele proportion score is the proportion of genomic variants that exceeds an incidence of 0.0001 in the plurality of genomes at the corresponding position.

In certain embodiments, described herein, are systems to identify the relative genomic health risk of a genomic sequence variant of an individual comprising: a DNA sequence for the individual; a system to determine at least one genomic sequence variant in the DNA sequence of the individual; wherein the genomic sequence variant is a difference of at least one nucleotide in the individual when compared to a corresponding position in a reference genome in a unique sequence of n nucleotides in length; and a system to determine an n-variant score for the at least one genomic sequence variant, wherein the n-variant score is comprises a function of a count score and an allele frequency score, wherein the count score is the ratio of the number of times any genomic sequence variant occurs in a unique sequence of n-nucleotides in length in the plurality of genomes to the number of times that the unique sequence of n-nucleotides in length occurs in the reference genome, and the allele frequency score is the frequency of the proportion of genomic sequence variants that are fixed in the population, at an allele frequency greater than 0.0001 in the plurality of genomes.

In certain embodiments, described herein, are systems to identify the relative genomic health risk of a genomic sequence variant of an individual comprising: a DNA sequence for the individual; a system to determine at least one genomic sequence variant in a DNA sequence of the individual; wherein the genomic sequence variant is a difference of at least one nucleotide in the individual when compared to a corresponding position in a reference genome; and a system to determine if the at least one genomic sequence variant occurs within a region with a low context dependent tolerance score, wherein the context dependent tolerance score comprises a function of an observed context dependent tolerance score and an expected context dependent tolerance score, wherein the expected context dependent tolerance score is the overall probability to vary of a unique sequence of n-nucleotides in length in a certain region of x nucleotides in length actually observed and fixed in a plurality of genomes, and the observed context dependent tolerance score is a number of genomic sequence variants in a certain region of x nucleotides in length actually observed in the plurality of genomes.

In certain embodiments, described herein, are systems to identify the relative genomic health risk of a genomic sequence variant of an individual comprising: a DNA sequence for the individual; a system to determine at least one genomic sequence variant in a DNA sequence of the individual; wherein the genomic sequence variant is a difference of at least one nucleotide in the individual when compared to a corresponding position in a reference genome; a system to determine if the at least one genomic sequence variant causes an amino acid variant in an expressed protein, wherein the amino acid variant is a difference of at least one amino acid when compared to a reference genome; and a system to compare the amino acid variant to a protein tolerability score at a corresponding position within a defined protein class, wherein the protein tolerability score comprises a diversity score, missense score, and a protein allele frequency score, wherein the diversity score is a normalized diversity metric, the missense score is the variance observed in a plurality of genomes at the corresponding position which leads to an amino acid mutation, and the protein allele frequency score is the proportion of genomic variants that leads to an amino acid variant that exceeds an incidence of 0.0001 in the plurality of genomes at the corresponding position.

### EXAMPLES

The following examples are illustrative and not meant to limit this disclosure in any way.

### High Quality Sequencing of 10,000 Genomes

In an effort to evaluate the capabilities of whole human genome sequencing on the HiseqX platform, we first measured accuracy and generated quality standards by replica analyses of the reference genome NA12878 from the CEPH Utah reference collection (also known as “Genome-In-A-Bottle”, GiaB). We then assessed these quality standards across 10,545 human genomes sequenced to high depth. This allowed for the development of a reliable representation of human single nucleotide variation, and the reporting of clinically relevant single nucleotide variants (SNV) using new high throughput sequencing technology.

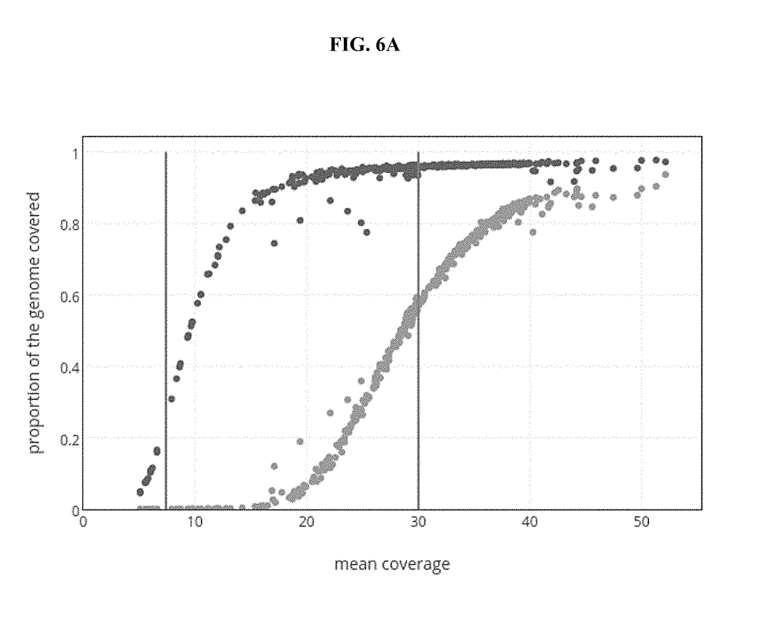

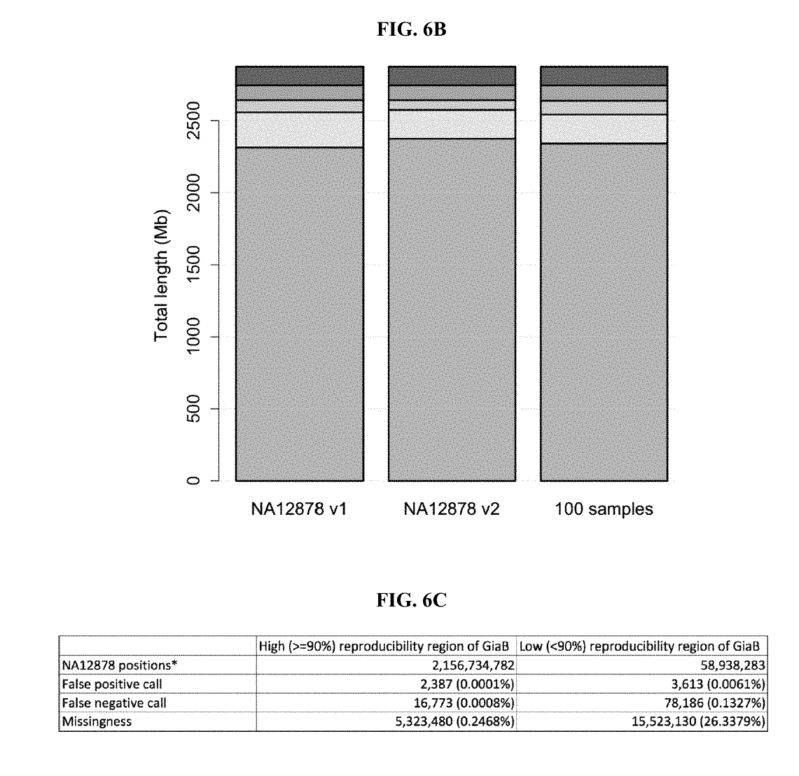

We first assessed the extent of genome coverage and representation using the data from 325 technical replicates of NA12878 at different depth of read coverage. We evaluated the accuracy and precision of the laboratory and computational processes to define quality metrics that might be applied to other samples to ensure consistent data quality. At the target mean coverage of 30×, 95% of the NA12878 genome is covered at least at 10×. In contrast, FIG. 6A shows that at a target mean coverage of 7× used by several genome projects, only 23% of NA12878 is sequenced at an effective 10×.

We next assessed reproducibility on variant calling for the whole genome by restricting the analysis to a set of 200 samples of NA12878 that were sequenced at a mean coverage of 30× to 40×. Due to manufacturer's changes in clustering reagents, we analyzed 100 samples prepared with v1 (original kit) and 100 with v2. In FIG. 6B, after applying quality filters, passing genotypes (i.e., those with a PASS call in the variant call format [VCF] file) were compared for consistency. For v2 chemistry, 2.51 billion positions passed, and were called with 100% reproducibility in all replicates. Similarly, 2.44 billion positions passed for v1. An additional 210 Mb of genome positions yielded passing reproducible genotypes in more than 90% of samples for v2 chemistry and 258 Mb for v1 chemistry. Only 184 Mb of genome positions were sequenced with lower reproducibility (<90%). The analysis of 100 unrelated genomes (25 individuals for each of the three main populations, African, Asian, European, and 25 admixed individuals) confirmed the consistency of calls across the genome.

The canonical NA12878 Genome-In-A-Bottle call set (GiaB v2.19) defines a set of high confidence regions that corresponds to approximately 70% of the total genome. The data for this GiaB high confidence region are derived from 11 technologies: BioNano Genomics, Complete Genomics, Ion Proton, Oxford Nanopore, Pacific Biosciences, SOLiD, 10× Genomics GemCode WGS, and Illumina paired-end, mate-pair, and synthetic long reads. Regions of low complexity (e.g., centromeres, telomeres and repetitive regions) as well as other regions that have proven challenging for sequencing, alignment and variant calling methods are excluded from the GiaB high confidence region. The above analysis of reproducibility addressed the whole genome of NA12878—both in the GiaB high confidence region, and beyond those boundaries. We thus used the reproducibility metrics to define regions within GiaB with high (≧90%) versus low (<90%) reproducibility at each position. The reproducibility metrics include the concordance in calls and missingness (defined in this disclosure as a measure of no-PASS calls). FIG. 6C shows that a precise assessment of missingness is achieved by using a genomic variant call format file gVCF that informs every position in the genome regardless of whether a variant was identified at any given site or not. A total of 2,157 Mb (97.3%) of the GiaB high confidence region could be sequenced with high reproducibility, while 59 Mb (2.7%) were classified as less reliable. False positive, false negative and missingness rates were considerably lower in the GiaB region sequenced with high reproducibility. This suggests that, by defining high reproducibility sites, the false discovery rate is kept very low (FDR=0.0025, or 0.25%). Other relevant metrics included a Precision of 0.998, Recall of 0.980 and a F-measure of 0.989. Overall, these first analyses indicate that the current technology and sequencing conditions generate highly accurate sequence data over a large proportion of the genome.

### Defining High Confidence Regions for Analysis

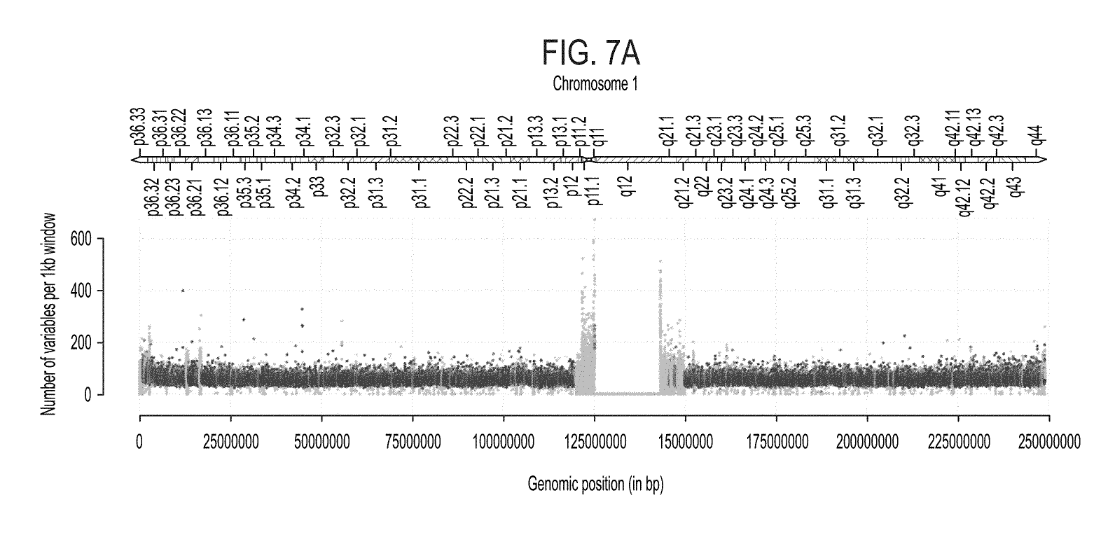

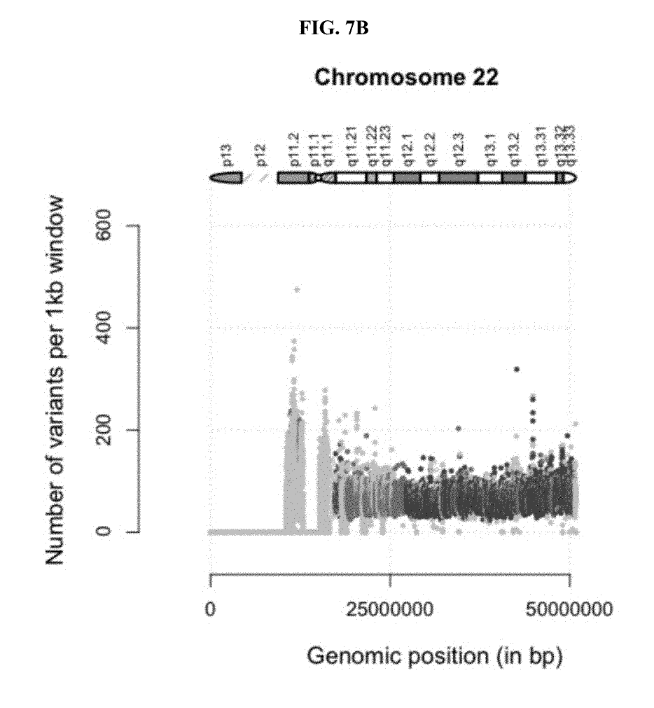

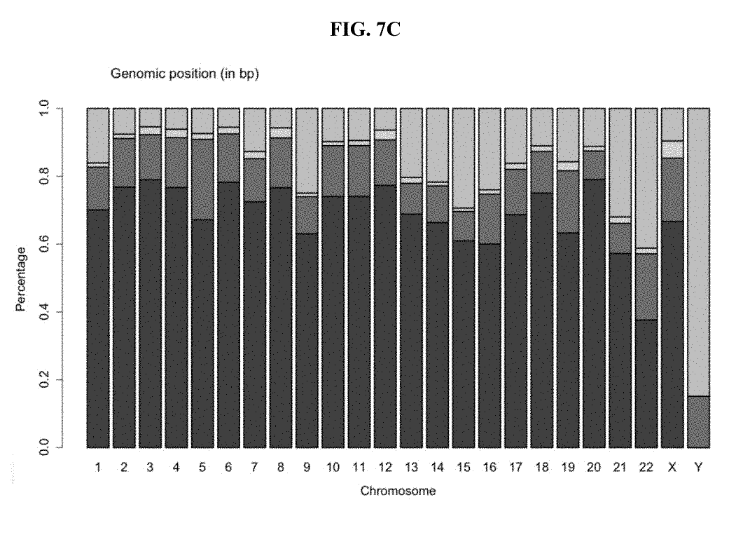

We next defined an extended confidence region (ECR) that includes the high confidence GiaB regions and the highly reproducible regions extending beyond the boundaries of GiaB. We also defined a low confidence region to include the regions within and beyond the boundaries of GiaB that could not be sequenced reliably with the technology in use. FIGS. 7A and 7B illustrate the noise we observed outside of the GiaB regions, both in terms of spurious variant calls and of apparent conservation. Of 3,088 Mb of sequence (autosomal, X- and Y-chromosomes), in FIG. 7C the overlap of GiaB high confidence and highly reproducible regions represented 69.8% of the analyzed positions. FIG. 7C shows the non-GiaB regions with high variant call reproducibility covered an additional 14.1% of the genome. Therefore, the newly defined ECR encompasses 83.9% of the human genome, and it includes 91.5% of the human exome sequence (Gencode, 96 Mb), which is consistent with recent reports on coverage of the human exome in whole genome analyses. We also examined the relevance for clinical variant calls: 28,831 of 30,288 (95.2%) unique ClinVar and HGMD pathogenic variant positions are found in the ECR.

Creating Metaprofiles that Capture Human Variation

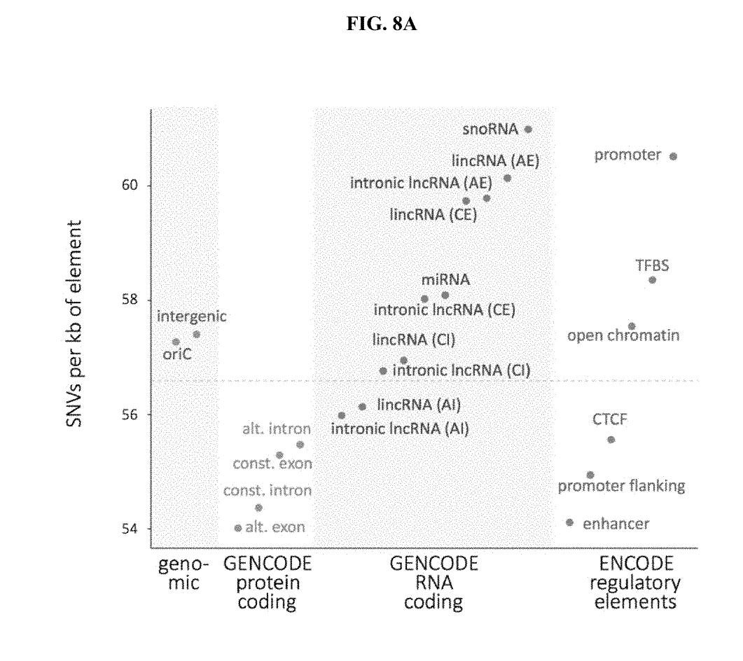

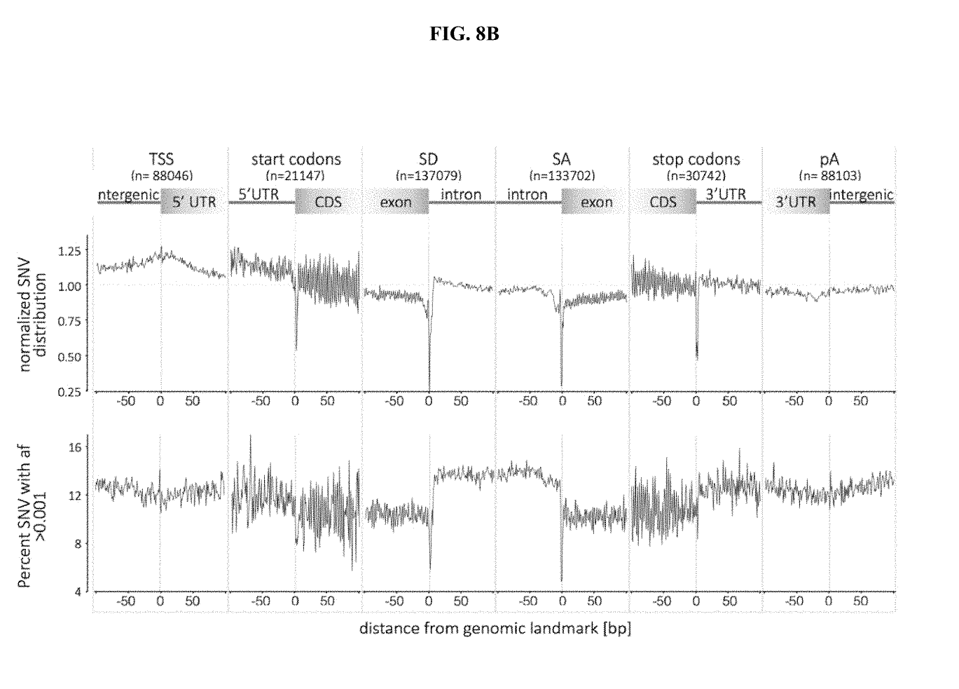

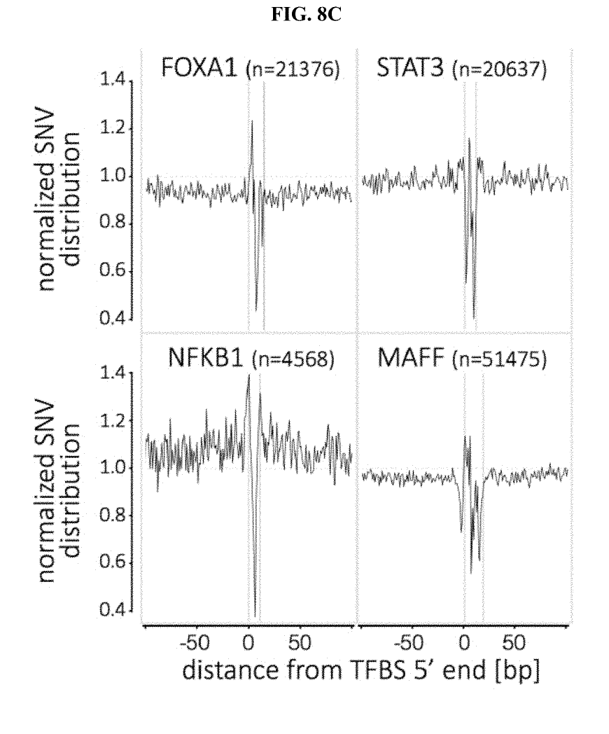

The volume of data presented here provides unprecedented detail on the pattern of sequence conservation and SNVs across the human genome. In FIG. 8A, we compared the rates of diversity in protein-coding, RNA coding, and regulatory elements. All protein-coding elements are more conserved than intergenic regions; as previously reported, alternative exons are the least variable. Alternative introns of lncRNAs are the most conserved and snoRNA the most variable of RNA coding elements. FIG. 8A shows that among the analyzed DNA regulatory elements, repressed chromatin are the most conserved, and transcription start site loci are the least conserved.

In order to explore the pattern of variation in the human genome in depth, we built “SNV metaprofiles” by collapsing all members of a family of genomic elements into a single alignment. Metaprofiles of protein-coding genes used GENCODE annotated TSS (n=88,046), start codons (n=21,147), splice donor and acceptor sites (n=137,079 and 133,702, respectively), stop codons (n=37,742) and polyadenylation sites (n=88,103). FIG. 8B shows that for each nucleotide aligned against these landmark positions, all of the genomes in this dataset (n=10,545) were used to generate a precise representation of the pattern of conservation, and allele spectra. The pattern is built by incorporating up to 1.4 billion data points (number of aligned elements×10,545 samples) per genomic position. For example, FIG. 8B shows the analysis captures the decrease in variant allele frequency in exons, with the maximum drop occurring at the splice donor site. In addition, the metaprofiles reveal emerging patterns, including with great precision the periodicity of conservation in coding regions due to the degeneracy of the third nucleotide in the codon in every exon window.

A second example of functional inference from patterns of variation is provided in FIG. 8C. Here we highlight the unique SNV metaprofiles at transcription factor binding sites. For this analysis, we use the binding site core motifs for landmarking. FIG. 8C shows metaprofile identify signatures that include both variation-intolerant and hyper-tolerant positions at the binding site. Positions that do not tolerate human variation can be interpreted as essential and possibly linked to embryonic lethality. While the identification of conserved, intolerant sites is expected, the biology behind unique hypertolerant positions at those sites remains to be investigated. Metaprofiles also register positions and domains that, while tolerant to rare variation, show limited possibility for fixation (allele frequencies are kept extremely low). We speculate that rare human variants in such domains carry a greater fitness cost, associate with greater phenotypic consequences and can be prioritized for clinical assessment.

### Example Validation of Tolerability Score for Predicting Harmful Genomic Sequence Variants

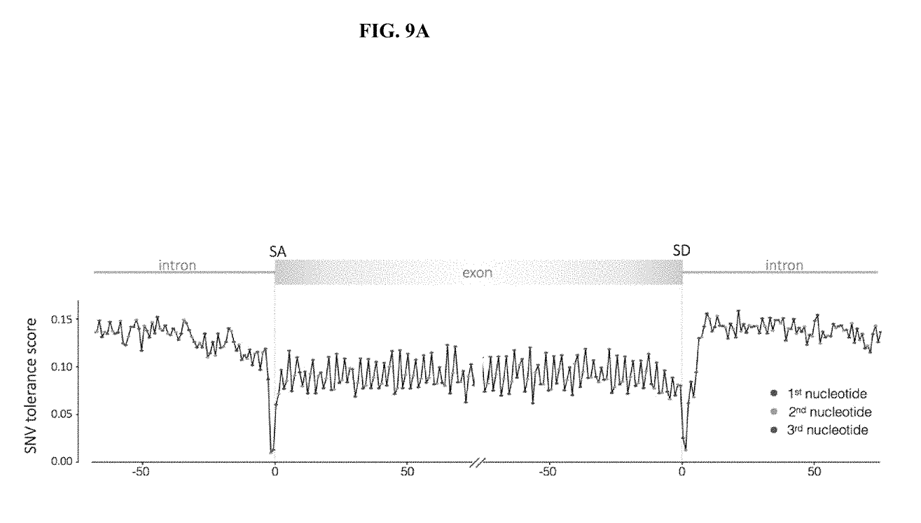

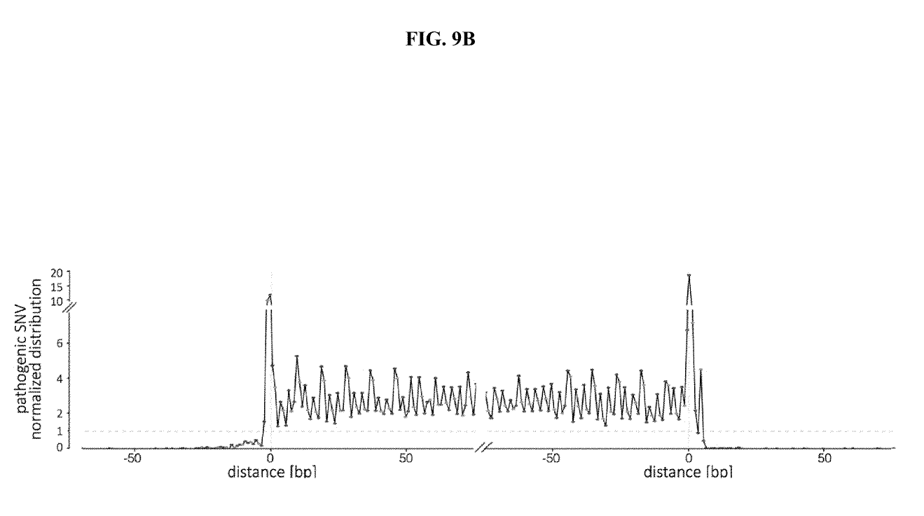

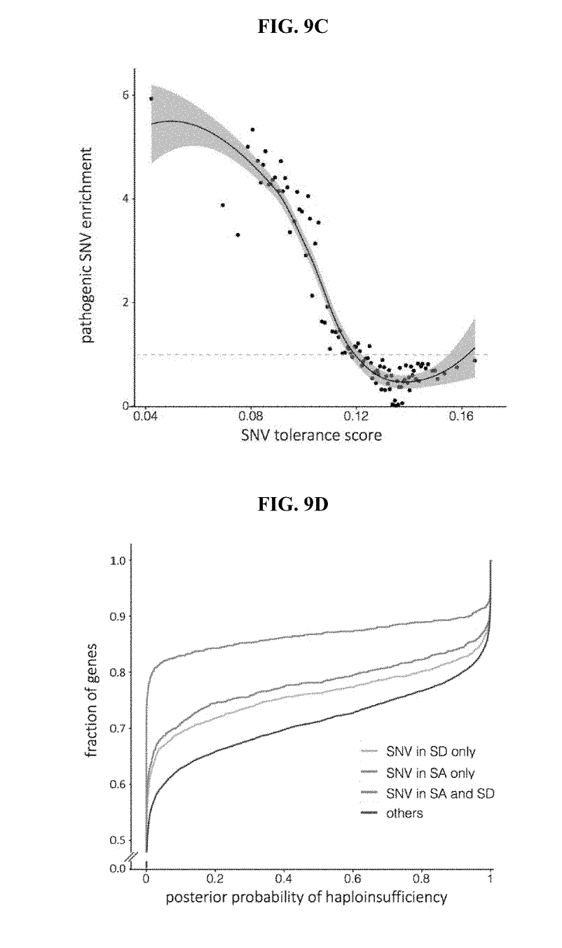

To assess the value of a tolerability score for scoring of functional severity of GSV, we established a tolerance score FIG. 9A that summarizes the rates and frequency of variation at a given position and for a given landmark. Using this approach, FIG. 9B illustrates the accumulation of pathogenic variant calls at sites with the lowest metaprofile tolerance scores. To formalize this analysis, FIG. 9C shows the tolerance score at 1,200 positions aligned to particular coding region landmarks: 100 positions upstream and downstream of the TSS, start codon, splice donor and acceptor, stop codon and polyadenylation site. At the lowest tolerance score, we observed up to 6-fold enrichment for pathogenic variants.

However, the assignment of pathogenicity or functional severity can be significantly biased by ascertainment (e.g., “it is at a splice site, it should then be a pathogenic variant”). In addition, variants are still observed at sites with very low metaprofile tolerance scores. In FIG. 9D, to understand the characteristics of genes that tolerate variants at those privileged sites we used an orthogonal assessment of gene essentiality. See Bartha et al., The Characteristics of Heterozygous Protein Truncating Variants in the Human Genome. PLoS Comput Biol 11, e1004647 (2015). The set of essential genes includes highly conserved genes that have fewer paralogs, and are part of larger protein complexes. Essential genes also display a higher probability of CRISPR Cas9 editing compromising cell viability, and knockouts in the mouse model are associated with increased mortality. FIGS. 9A-9D illustrate the concept that genes that tolerate variation at sites with low tolerance scores are less essential.

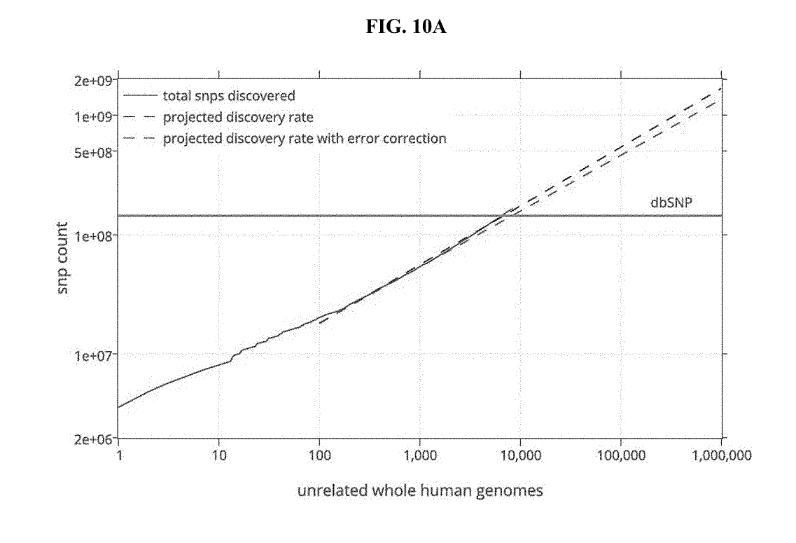

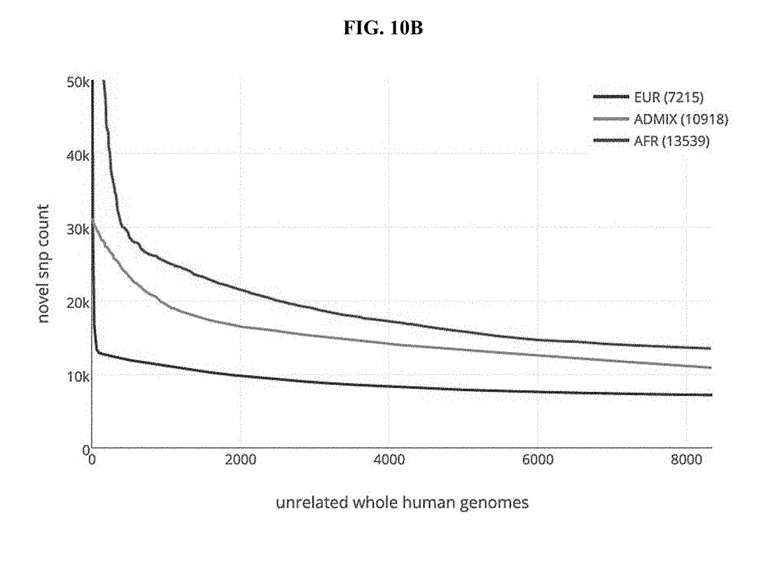

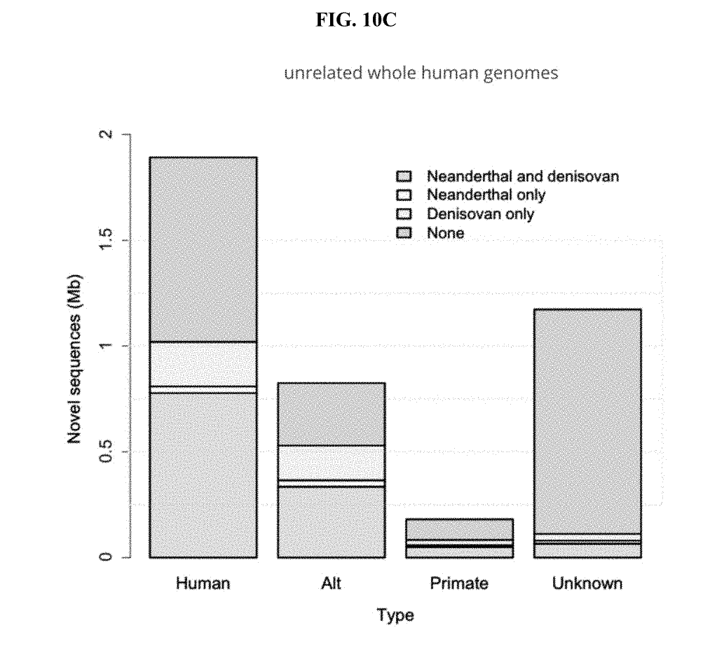

FIG. 10A shows that a large number of genomes, and a broad coverage of human populations served to describe the rate of newly observed, unshared SNVs for each additional sequenced genome. We restricted the analysis to the 8,137 unrelated individuals among the 10,545 genomes—as defined by an estimated kinship coefficient to exclude first degree relatives. In the absence of an earlier saturation of sites due to biological and fitness constrains, there is an expectation of 500 million variants identified after sequencing the genomes of 100,000 individuals.

In FIG. 10B, unrelated individuals were assigned to five superpopulations as described by The 1000 Genomes Project, or to an admixed or “other” population group on the basis of genetic ancestry (EUR, n=5,596; AFR, n=962; SAS, n=62; EAS, n=148; AMR, n=12; ADMIX, n=1,288; other, n=57). FIG. 10B shows that each subsequently sequenced genome contributes on average 8,579 novel variants. For the three populations represented by >900 individuals, the number of newly observed unshared variants per sample varied from 7,214 in Europeans and 10,978 in admixed, to 13,530 in individuals of African ancestry This reflects the current understanding of Africa as the most genetically diverse region in the world. Of the 150 million SNVs observed in the ECR, 82 million (54.7%) have not been reported in db SNP of the National Center for Biotechnology Information.

Much of the non-reference sequence is shared with hominins. In FIG. 10C, the unmapped contigs were compared to Neanderthal and Denisovan sequencing reads that did not map to hg38. There were 809 contigs (0.96 Mb) covered by Neanderthal reads and 999 contigs (1.18 Mb) covered by Denisovan reads. In addition, we identified 608 contigs (0.82 Mb) that are not in hg38 primary assembly, but in the “alt” sequences or subsequent patches. Those contigs are not included in the above estimates of non-reference sequence. Collectively, we observed over 3Mb of sequence that is not represented in the main hg38 build and “alt” sequences.

CDTS Defines Pathogenic Sequence Variance Better than Methods that Use Inter Species Conservation

Traditionally, conservation in the genome has been identified through the comparison among species: if a segment of genome is conserved across many species, then it is assumed that it is important. Therefore, to compare the conserved human genomics regions as defined by a context dependent tolerability score (CDTS) with findings in the larger context of interspecies conservation, we assessed the extent of overlap of conserved regions assessed with CDTS (i.e., context-dependent conservation in the current human population) and Genomic Evolutionary Rate Profiling (GERP) across 34 mammalian species (i.e., interspecies conservation). From the 1^{st} to 10^{th} percentile levels, the overlap between both scores is limited and heavily enriched for protein-coding regions. FIGS. 11A and 11B show results from these experiments. FIG. 11A shows the composition in the first percentile regions by CDTS (the bar labelled as “CTDS 1^{st}”), GERP (“GERP 1^{st}”) and the overlap region of CDTS and GERP (“Intersection”), as defined by functional genomic elements. The data shows that there is little overlap between highly conserved regions as defined by CDTS and GERP, outside of protein-coding exons. FIGS. 11C and 11D show that the overall length of the genome that falls into the 1^{st} percentile by CDTS and GERD overwhelming indicates that there is very little overlap between the two methods in identifying highly conserved sequences outside of protein-coding exons. FIG. 11C shows an analysis as in FIG. 11A except the 1^{st} to the 10^{th} percentile is analyzed. FIG. 11D shows an analysis as in FIG. 11B except the 1^{st} to the 10^{th} percentile is analyzed. Surprisingly, these results suggest that the least variable non-coding regions in human populations are primarily revealed by CDTS and not by an interspecies evolutionary relationship.

### Genomes

The analysis used deep sequence genome data of 11,257 individuals. Analysis was limited to the high confidence region of the genome (as defined in Telenti, A. et al. “Deep sequencing of 10,000 human genomes,” Proc Natl Acad Sci USA) a region covering approximately 84% of the genome and closely overlapping with the high confidence region as described in the most recent release of Genome in a Bottle (GiaB v3.2).

### Metaprofiles

Metaprofiles comprise the massive alignment of elements of the same nature in the genome. These genomic elements can be chosen based on their structure (e.g., exonic, intronic, intergenic, etc.), function (e.g., transcription factor binding sites, protein domains, etc.) or sequence composition (k-mers). Genetic diversity is assessed at each nucleotide position of the alignment of genomic elements, by monitoring both the occurrence of variation in the population (reported as a binary—presence or absence) and the allelic frequency. More specifically, 3 metrics are computed at each position: (i) the percent of elements with SNVs,(ii) the percent of SNVs with an allelic frequency higher than 0.001 or 0.0001, and (iii) the product of both scores. Each score is calculated using between 10⁶ and 10¹⁰ values, a value provided by the number of elements present in the genome and aligned multiplied by the number of genomes sequenced; therefore, the metaprofile strategy massively increases the power to compute variation rate at nucleotide resolution with high precision. A priori knowledge of genomic landmarks is required for constructing metaprofiles based on similarity in structure or function. In order to remove potential biases through the use of this a priori knowledge, we developed a strategy to construct metaprofiles based on all possible heptameric sequences found in the genome (4⁷=16384) and scored the middle nucleotide for each of these sequences as described above. As every nucleotide in the genome is part of an heptamer, every single position can be attributed to the corresponding genome-wide computed scores. Scores are computed separately for autosomes and chromosome X. To account for the difference in effective population size over history for chromosome X, the allelic frequency threshold is adjusted by a factor of 0.75. In a certain aspect, indels are not used to compute the score. When testing the score on smaller study populations the allelic frequency threshold was adjusted to retain only non-singleton positions.

### Expected Versus Observed

The variation rates computed through heptamer metaprofiles reflect the chemical propensity of a nucleotide to vary depending on its surrounding context and can be interpreted as an expectation of variation. We rationalized that functional regions would vary significantly less than they would be expected to, as assessed genome-wide through the heptamer tolerance score. To evaluate the departure from expectation, we compared the observed and expected tolerance score obtained in defined genomic regions.

The observed regional tolerance score is the number of SNVs present at an allelic frequency higher than 0.001 in the studied population in a defined region. The expected regional tolerance score is the sum of the heptamer tolerance scores in the same region.

The difference between the observed and expected scores is further referred to as context-dependent tolerance score (CDTS). The regions are then ranked based on their CDTS. The regions with the lowest rank are the regions with the lowest context-dependent tolerance to variability and the regions with the highest rank are the regions with the highest context-dependent tolerance to variability. Genomic regions are ranked based on their CDTS. Regions with the lowest rank (1^{st} percentile) have the lowest context-dependent tolerance to variation. Regions with the highest rank (100^{th} percentile) have the highest context-dependent tolerance to variation.

### Region Definition and Annotation

To avoid any use of a priori knowledge and any biases due to the differing size of the regions (i.e., more power to detect difference between observation and expectation in longer elements), the genome was chopped irrespective of genomic annotations into sliding windows of the same size. The window size was 1050 bp sliding every 50 bp and the calculated CDTS across the 1050 bp window was attributed to the middle 50 bp bin. Only regions with at least 90% of the nucleotides in the 1050 bp window present in high confidence regions were used. To evaluate the element distribution across those size defined windows, we built a new annotation model by combining sources of annotation from GenCode (v.23) and ENCODE (annotated features and multicell regulatory elements, Ensembl v84 Regulatory Build). In order to avoid conflicting and overlapping annotations from the two different sources and thereby use the score of the same region multiple times, we prioritized element annotation as follows, such that only the highest order element would be used: exonic, then multicell, then intronic and then annotated features. We assessed the element composition of the different percentiles, using the above mentioned combined GenCode/ENCODE annotation, by computing the number of nucleotides of an element in each percentile. The following categories were used: “Exon—protein coding”, referring to nucleotides in exonic regions contained in protein-coding genes (including UTR) as annotated in GenCode; “Exon—non-coding”, referring to nucleotides in exonic regions contained in non-coding RNAs (e.g., snRNA, snoRNA, lincRNA, etc.) as annotated in GenCode; “Intron”, referring to nucleotides in intronic regions contained in either protein-coding or non-coding genes as annotated in GenCode; “Promoter”, “Promoter Flanking” and “Enhancer”, referring to the nucleotides contained in the respective elements as annotated in ENCODE multicell regulatory elements; “H3K9me3” and “H3K27me3”, referring to the nucleotides overlapping with (and only) the respective elements as annotated in ENCODE annotated features; “Multiple Histone marks”, referring to the nucleotides overlapping with a combination of histone marks, as annotated in ENCODE annotated features; “Others”, referring to the remaining nucleotides with ENCODE annotated features that did not cover a substantial part of the genome individually, which notably encompasses transcription factor binding sites as well as other regulatory element combinations (e.g., nucleotides annotated as both Promoter and Enhancer); and “Unannotated”, referring to nucleotides in regions that had no annotated features in either GenCode or ENCODE.

### Essentiality and CDTS Coordination

We used gene essentiality (pLI score from ExAC²) as an orthogonal proxy for functionality to assess whether genomic bins, annotated with the same genomic element, have different biological importance depending on their CDTS ranking. Each genomic bin present within 10 kb of a gene is attributed the essentiality score of its closest or overlapping gene, with the exception of genomic bins annotated as “Promoters,” that have the mandatory constraint of being upstream of the closest gene. The median essentiality score is then assessed per genomic element annotation and per percentile slice. To assess distal CDTS coordination, we used an external chromatin loop dataset. The loop and anchor coordinates were extracted from previous Hi-C experiment. The median CDTS percentile is computed for every anchor region. To pair distal enhancers with their hypothetically associated genes, for each loop we extracted the genes and enhancers that were the closest to both loop-anchor points. We then kept only meaningful pairs, where an enhancer was annotated in the upstream anchor and a gene in the downstream anchor, or vice versa. In addition, the 5 prime end of the gene had to be facing the loop. A maximum of one pair per gene was retained; in the cases of several possible pairs, the pair was kept that had the smallest total distance between the enhancer to the gene after subtracting the loop size. We computed the median CDTS of the enhancers associated in such a distal gene-enhancer pair and compared it to the essentiality score of the associated gene.

### Interspecies Conservation

We used Genomic Evolutionary Rate Profiling (GERP++) to capture the interspecies conservation. GERP++ provides conservation scores through the quantification of position specific constraint in multiple species alignments. We calculated and attributed the mean GERP scores to the same set of 50 bp bins as mentioned in the section “Region definition and annotation.” Bins were ranked based on the GERP score from the most (percentile 1) to the least conserved (percentile 100). Bins without GERP score, due to insufficient multiple species alignments in the region, were not considered in the ranking process.

### CDTS Reveals a Previously Unknown Additional Novel Level of Conservation in the Human Genome

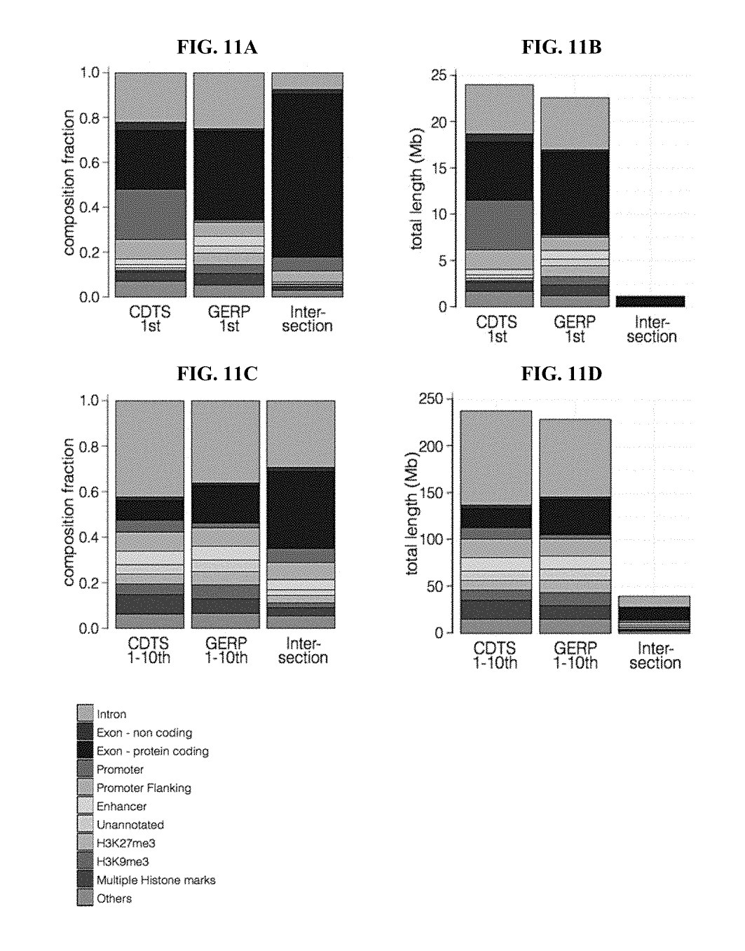

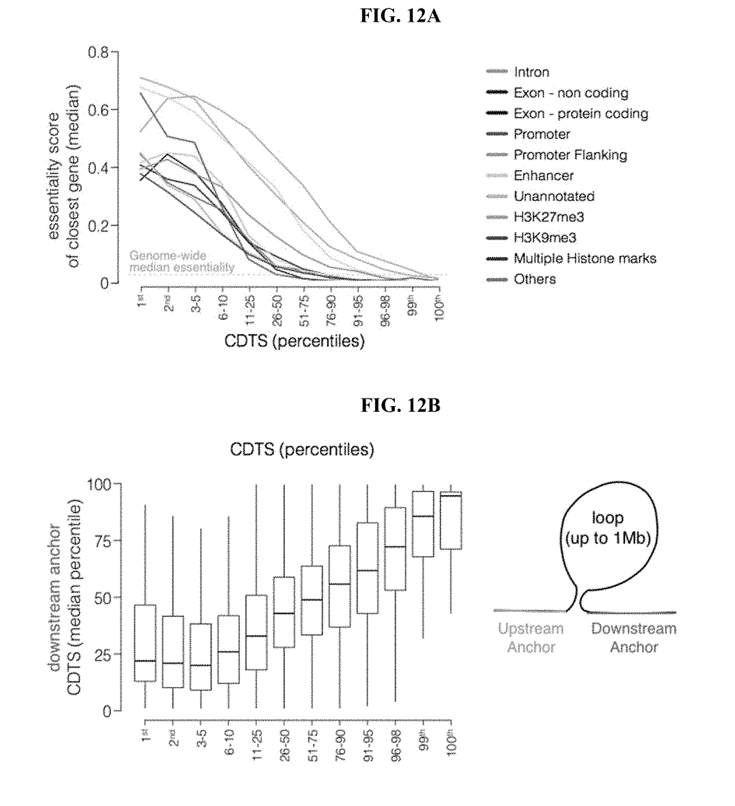

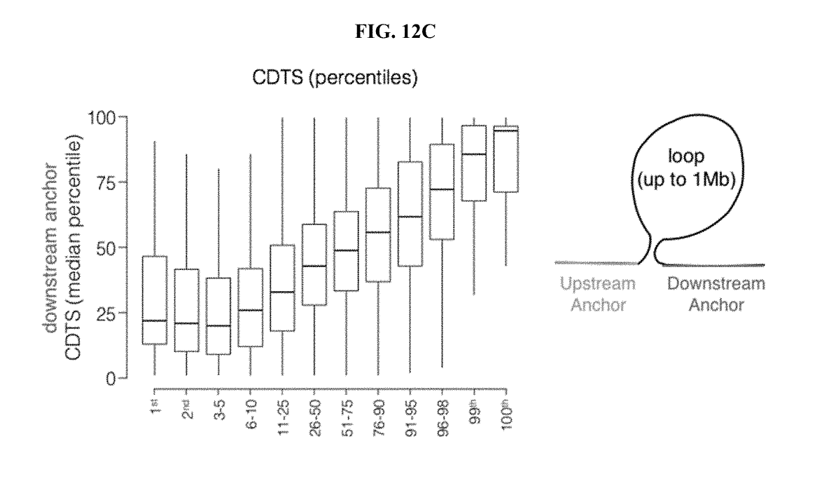

A surprising result emerges from the mapping of all human conserved regions as represented by CDTS. The genome structure that is revealed is one of coordination of genes with the respective regulatory regions. For example, a very important gene (“essential gene”) will use a very conserved promoter, cis enhancer, distal regulatory elements and other regulatory signals. This new data provides enhanced ability to pair the genes with the generally under- or un-recognized regulatory units, which is key to understanding function in health and disease. This also allows for using CDTS to identify pathogenic variants, and to build a targeted sequencing and genotyping array for diagnostics. As expected, FIG. 12A shows exons in essential genes were enriched in the conserved regions of the genome as defined by CDTS. We first assigned the essentiality score of the gene to the corresponding upstream promoter. This analysis confirmed that promoters in the conserved part of the genome associate with essential genes. We then observed that cis enhancer regions also shared sequence conservation with genes (within 10 kb) that were putatively regulated by those elements as shown in FIG. 12A. Next, we searched for evidence that functional constraints could be shared over greater distances. Topological associated domains were defined using information from Hi-C and 3D genome structure data. We observed that the regions brought together through these long-distance interactions shared similar levels of conservation as reflected by the CDTS values. FIG. 12B shows that this this coordination was maintained at distances as long as one megabase. In addition, and despite the complexity to associate distant regulatory regions with a particular gene, FIG. 12C shows that we observed a correlation between conservation of the distal enhancer, and the essentiality of the putative target gene. Finally, we assessed other cis non-coding elements (e.g., chromatin histone marks, transcription factor binding sites), and unannotated and intronic regions, and consistently identified a pattern of correlation between conservation scores of non-coding or regulatory regions with gene essentiality. Strikingly, FIG. 12A confirms that even genomic elements that were depleted in the most conserved part of the genome (e.g., H3K9me3 and H3K27me3) are associated with essential genes when present in the lower CDTS percentiles. More generally, regions of low CDTS appear clustered in the genome. Overall, the data support the concept of conserved and coordinated regulatory and coding units in the genome over large genome distances.

### Distribution of Pathogenic Variants Across the Genome

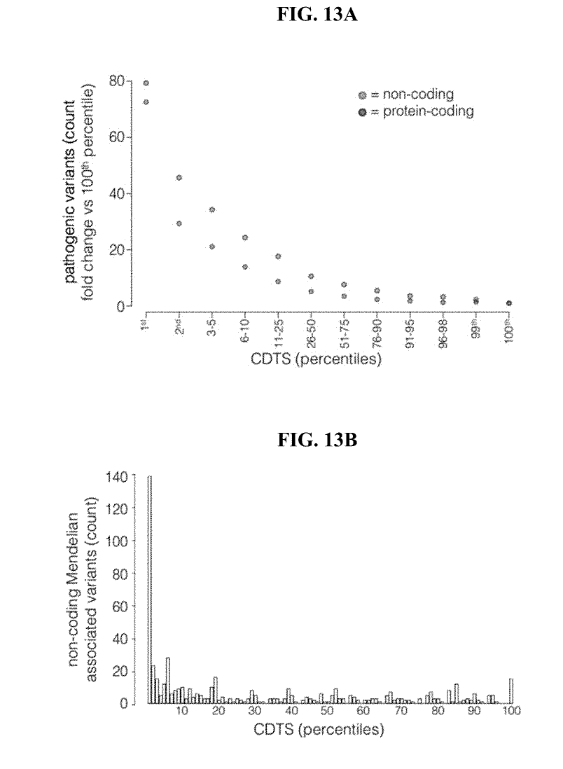

The description of the conserved genome raises the issue of its relevance to human disease. We assessed whether CDTS ranking was a good proxy to score functional constraint and the consequences of mutations. For this purpose, we investigated the distribution of annotated pathogenic variants across the genome. FIG. 13A shows that the pattern of enrichment was marked for pathogenic variants in the 1^{st} versus the 100^{th} percentile for both protein-coding (73-fold) and, more importantly, for non-coding (79-fold) pathogenic variants. Of note, the enrichment of non-coding pathogenic variants is even more striking after accounting for the size of the non-coding territory covered in each percentile slice and reaches >100-fold enrichment. To confirm these findings, we further investigated 550 manually curated non-coding variants associated with 118 Mendelian disorders. We confirmed that Mendelian non-coding variants are highly enriched in the regions with the lowest CDTS values as shown in FIG. 13B. Table 1 lists the 1,000 lowest percentile (most conserved) non protein-coding variants by genomic position as defined by CDTS. Table 2 lists the lowest percentile (most conserved) non protein-coding known SNPs by genomic position as defined by CDTS.

### Pathogenic Variants

We assessed the distribution of known annotated pathogenic variants, defined as either HGMD high DM 14 (Version: HGMD_2016_R1) or ClinVar variants consistently annotated as pathogenic or likely pathogenic and with at least 1 entry with star 1 or more15,16 (Version: ClinVarFullRelease_2016-07.xml.gz) for a total N=130,767, by counting the number of variants present in each percentile of the genome. For variants in indel regions, the left most coordinate was used to establish in which genomic bin they fell. Pathogenic variants with conflicting annotations were removed, defined here as variants having a high DM in HGMD and a consistent annotation of benign or likely benign with at least 1 entry being star 1 or more in ClinVar. The non-coding variants associated with Mendelian traits were extracted from ClinVar (copy number variants were excluded from analysis) and manually curated with a filter of >5bp from any splice acceptor or splice donor site, and additional variants were collected by literature review 17-20.

### CDTS Identifies Pathological Variants

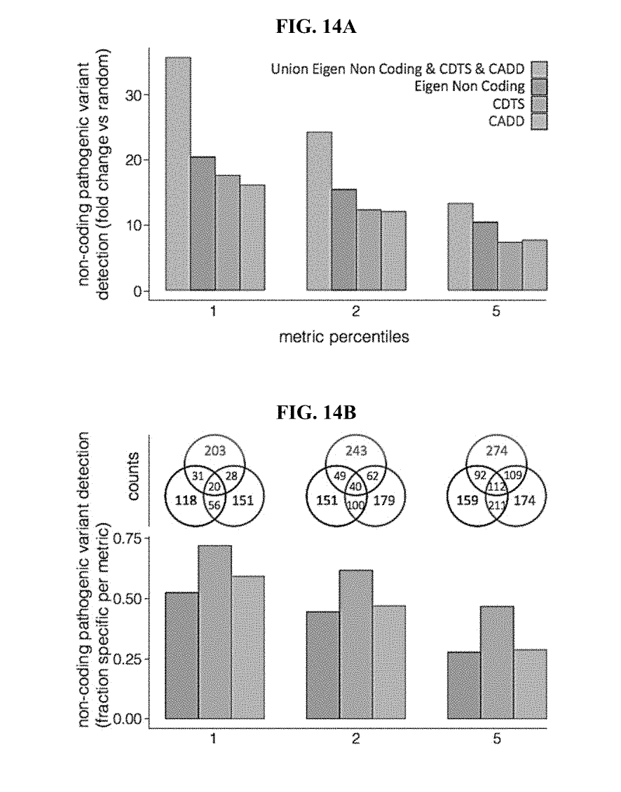

We explored how CDTS compared to other functional predictive scores used to prioritize variants, such as CADD and Eigen. We focused on the performance of these metrics on the non-coding genome. The combination of the three metrics provides the best detection, while the three metrics used alone provide similar ranges of detection as shown in FIG. 14A. As shown in FIG. 14B shows that CDTS is the functional predictive score that has the highest fraction of specific variant detection at any percentile threshold (barplot) providing high complementarity to the other metrics, while Eigen and CADD capture more redundant information (Venn diagrams). In addition, CDTS is the functional predictive score that detects the highest number of pathogenic variants, as the scores are computed for the whole genome, including sex chromosomes, and can be used for both SNVs and indels. Overall, CDTS requires no prior knowledge such as annotation or training sets, and captures a very specific set of pathogenic variants that are not detected by other metrics. Thus, CDTS complements other functional predictive scores in the analysis of the non-coding genome. Table 3 lists genomic positions that fall within the lowest 1^{st} percentile (most conserved) as defined by CDTS, and are unique to the CDTS method. Table 4 lists known SNPS that fall within the lowest 1^{st} (most conserved) percentile as defined by CDTS, and are unique to the CDTS method.

### Functional Predictive Scores

The CDTS metric was compared to the most widely used metrics for variant prioritization: CADD (Kircher, M. et al. A general framework for estimating the relative pathogenicity of human genetic variants. Nat Genet 46, 310-5 (2014)) and Eigen (Ionita-Laza, I., McCallum, K., Xu, B. & Buxbaum, J. D. A spectral approach integrating functional genomic annotations for coding and non-coding variants. Nat Genet 48, 214-20 (2016)). A “control” set of variants relative to the previously defined pathogenic variants was created using variants from dbSNP (June 2015 release). A control variant was defined as having the “COMMON” and “GSA” tag (>5% minor allele frequency in each population and all populations overall) and, similar to the tested pathogenic variant set, not be present in an exonic region and appear more than 5 bp from any splice site. The remaining working set of non-coding pathogenic and control variants were ranked according to their CDTS, CADD or Eigen non-coding scores and the ranking was normalized from 0 to 100 (for CADD and Eigen, the PHRED scores were converted into probabilities before this step, so that for all metrics the lower the ranking the more likely pathogenic a variant would be). To compare the different metrics, the precision (TP/(TP+FP)) was computed at each step of the new ranking. TP are the true positives, in this case the number of pathogenic variants with a ranking ≦threshold, and FP are the false positives, in this case the number of control variants with rank ≦threshold; where threshold can be any step in the new ranking (from 0 to 100). The precision was further normalized by the general prevalence of pathogenic variant in the set studied (Σ pathogenic/(Σpathogenic+Σcontrol)). This step was done in order to account for the fact that not all variants were scored by the other metrics (e.g., no scores on chromosome X for Eigen, conversion conflicts from hg19 to hg38, not all indel have a CADD score, etc.). The prevalence normalized precision provides the enrichment of a metric pathogenic variant detection compared to random.

### CDTS Identifies Unique Pathological Variants Compared to Other Metrics for Determining Pathogenicity

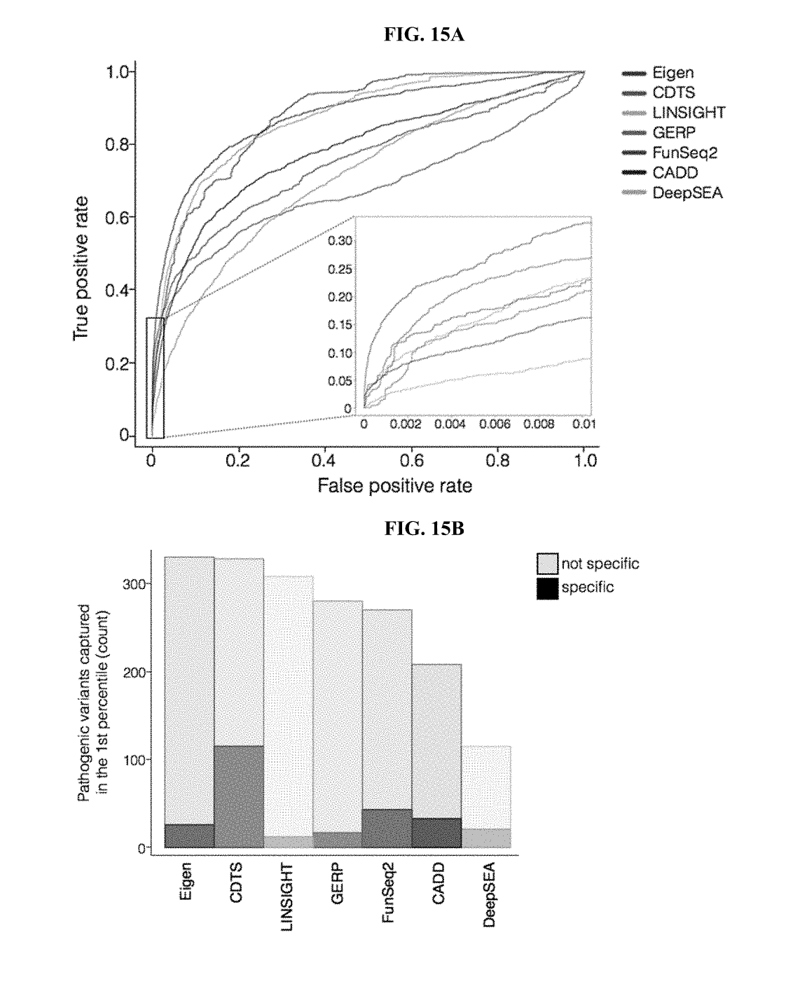

We explored how CDTS compared to other functional predictive scores used to prioritize variants in the non-coding genome, CDTS, Eigen, CADD, DeepSEA, GERP, funseq2, and LINSIGHT. To avoid the contribution of pathogenic variants in the proximity of exons, we focused the analysis to the stringent set of 1,369 non-coding pathogenic variants that were further than 10 bp from any splice site. Eigen and CDTS had the best performance of the metrics as represented by ROC curves as sown in FIG. 15A. Of the set of 1,369 non-coding pathogenic variants, 713 were identified by at least one of the metrics as being in their top 1st percentile score as sown in FIG. 15B. CDTS captures the highest proportion of variants only detected by a single metric (FIG. 15B). Other metrics capture more redundant information because they were developed or trained on similar datasets. In contrast, CDTS requires no prior knowledge such as annotation or training sets, and thus captures a very specific set of pathogenic variants.

### Methods

The CDTS metric was compared to other metrics used for variant prioritization: CADD, Eigen, GERP, DeepSEA, LINSIGHT and FunSeq2. A control set of variants relative to the previously defined pathogenic variants (N=1,369, detailed in the above paragraph) was created using variants from dbSNP ³′ (June 2015 release). The control variants were defined as having the “COMMON” and “G5A” tag (>5% minor allele frequency in each population and all populations overall, as well as in our own study population), being in high confidence region¹ and, similar to the tested pathogenic variant set, not be present in an exonic region and more than 10 bp from any splice site. The remaining working set of non-coding pathogenic and control variants were ranked according to their CDTS, CADD, Eigen, GERP, DeepSEA, LINSIGHT or FunSeq2 scores and the ranking was normalized from 0 to 100 (the direction of values of the scores were modified so that, for all metrics, the lower the rank would represent the pathogenic state. Of note, the CDTS ranking might differ slightly as only variant positions (control+pathogenic) are used here. To compare the different metrics, the true positive rate (TP/(TP+FN)) and false positive rate (FP/(FP+TN)) was computed at each step of the new ranking. TP are the true positives, in this case the number of pathogenic variants with a ranking ≦threshold; FP are the false positives, in this case the number of control variants with rank ≦threshold; FN are the false negatives, in this case the number of pathogenic variants with a ranking >threshold; TN are the true negatives, in this case the number of control variants with rank >threshold; where threshold can be any step in the new ranking (from 0 to 100). Given the fact that the control set of variants (N>5 mio) is order of magnitudes bigger than the pathogenic set (N=1,369), a false positive rate of 0.01 (threshold used in FIG. 15A for the zoom in view) corresponds approximately to the 1^{st} percentile of the data. Of note, not all variants were scored by all the metrics (e.g., no scores on chromosome X, conversion conflicts from hg19 to hg38, indels are not scored by all metrics, not in high confidence region, etc.). The number of non-coding pathogenic variants scored per metric are the following: CDTS (N=1,226), Eigen (N=1,000), CADD (N=1,283), DeepSEA (N=1,324), LINSIGHT (N=1,350), GERP (N=1,354) and FunSeq2 (N=1,203).

### CDTS Identifies Misidentified Genomic Features

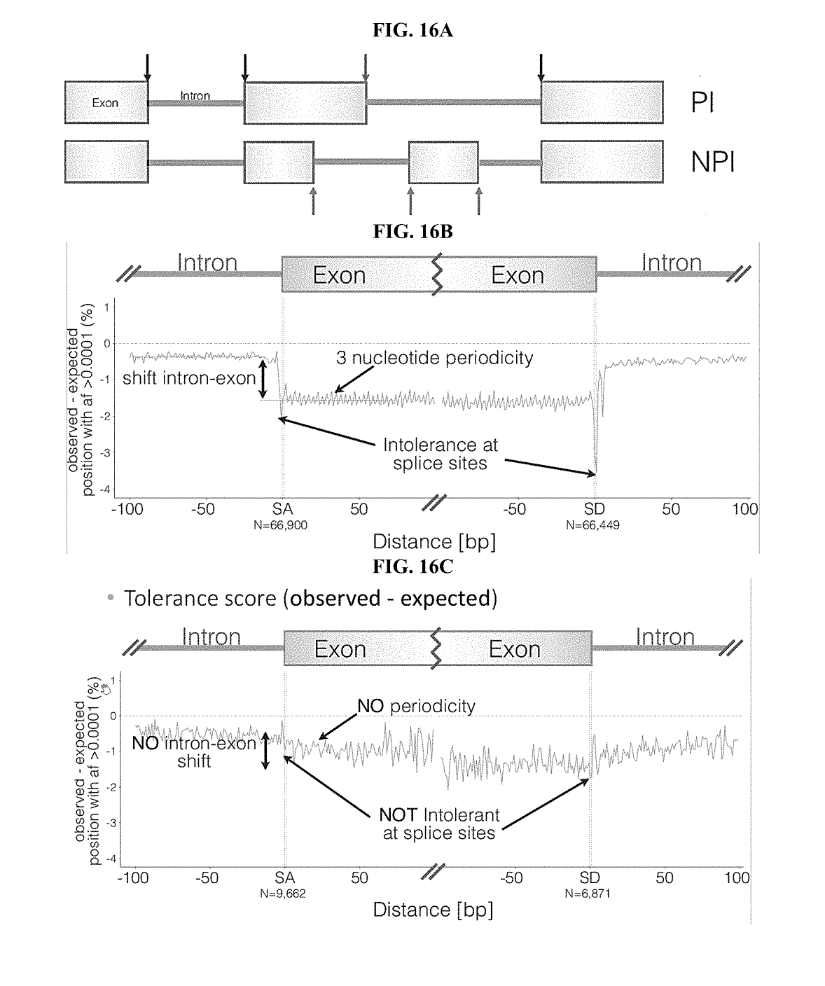

This example shows how metaprofiles and heptamer content analysis identifies new genomic elements that were misannotated so far. In short, we investigated 3 sets of splice sites described in FIG. 16A: (1) sites used only by the principal isoforms; (2) sites used by both principal (PI) and non-principal isoforms (NPI); and (3) sites used only by non principal isoforms We used CTDS tools to investigate whether the 3 groups behave differently (in reality represent different genomic elements)

Results: While the 2 first sets (present in the principal isoforms) behave similarly, the set of sites that are present only in non-principal isoforms do not show the characteristics of exon-intron junctions in terms of tolerance to variation as assessed by metaprofiling (FIG. 16B principal isoforms and FIG. 16C non-principal isoforms). In addition, the 3′UTR of the non-principal isoform, as well as their intronic region adjacent to the splice donors seem to display a different heptameric content than the respective regions in principal isoforms. Compared to other genomic features, the closest elements (in terms of heptamer content) to the 3′UTR of not-principal isoforms are long non-coding RNAs (lncRNAs). This could indicate that genome wide, there might be thousands of unannotated lncRNAs.

### CDTS Identifies Novel Pathogenic Variants

We assessed 6 candidate genes (POMC, LEP, LEPR, SIM1, MC4R, and PCSK1) that have previously been associated with early onset of obesity due to deficiency in the MC4R pathway, based on existing literature. To identify new pathogenic SNVs, we started by extracting all variants from a population of unrelated individuals (N=7794) that were found in the genes or vicinity (15 kb upstream and downstream) as well as in distal regulatory elements, as assessed by Hi-C and promoter-capture Hi-C. The criteria for an SNV to be candidate were the following: (i) the minimum BMI of the individual(s) carrying the alternative allele must be >=35; (ii) when applicable, individual(s) homozygous for the alternative allele must have a median Body mass index (BMI) higher than the median BMI of individual(s) heterozygous for the alternative allele; (iii) the SNV must be present in the population at an allelic frequency lower than 1/100; finally, (iv) the SNV must be “likely functional” as assessed by either one or more of the following metrics: CDTS, percentile <=2; CADD, score >=15; Eigen or Non-coding Eigen, score >=15; GERP, score >=5; Linsight, score >=0.8. The remaining SNVs are kept as candidates.

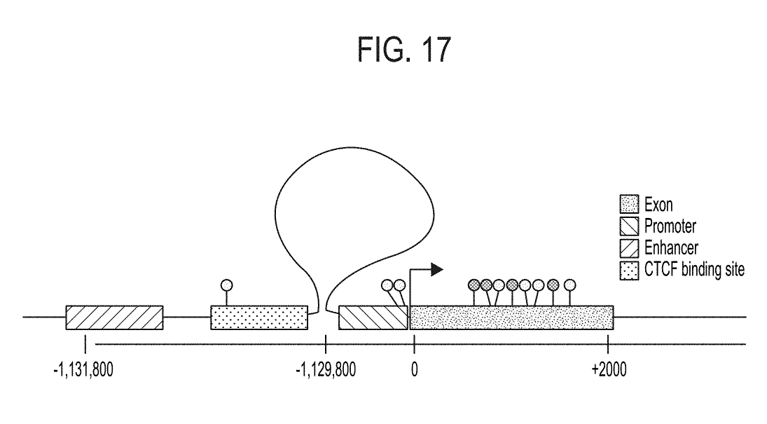

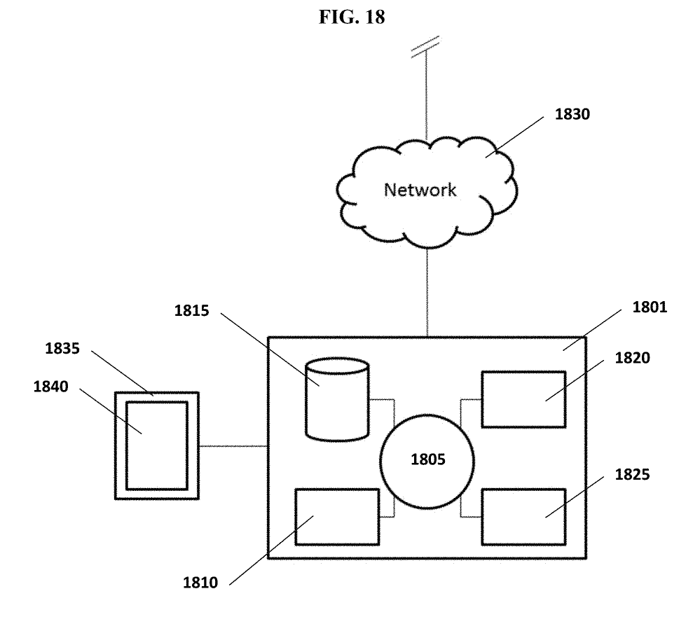

FIG. 17 illustrates candidate SNVs in MC4R gene and associated regulatory regions. The candidate variants associated with high BMI in the single exon gene, MC4R, are depicted as circles. The boxes represent genomic elements annotated in this genomic locus. The arrow indicates the transcription start site. Red colored circles are candidate variants that have previously been associated with high BMI (true positives) while yellow colored circles are candidate variants that are not known to be associated with high BMI (new candidates). Circles with a thicker edge weight indicate that the candidate variants are identified solely by CDTS. The coordinates indicate the distance (bp) between genomic elements.

Reports Generated and Delivered to Health Care Professionals and/or Consumers

Referring to FIG. 18, in a particular embodiment, an exemplary digital processing device 1801 is programmed or otherwise configured to calculate and/or organize a plurality of tolerability scores, n-variant scores, context dependent tolerability scores, or protein tolerability score s. The device 1801 can regulate various aspects of calculating and delivering the health risk metrics of the present disclosure, such as, for example, calculating one or more context dependent variability scores. In this embodiment, the digital processing device 1801 includes a central processing unit (CPU, also “processor” and “computer processor” herein) 1805, which can be a single core or multi core processor, or a plurality of processors for parallel processing. The digital processing device 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices 1825, such as cache, other memory, data storage and/or electronic display adapters. The memory 1810, storage unit 1815, interface 1820 and peripheral devices 1825 are in communication with the CPU 1805 through a communication bus (solid lines), such as a motherboard. The storage unit 1815 can be a data storage unit (or data repository) for storing data.

The digital processing device 1801 can be operatively coupled to a computer network (“network”) 1830 with the aid of the communication interface 1820. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the device 1801, can implement a peer-to-peer network, which may enable devices coupled to the device 1801 to behave as a client or a server. Reports can be delivered from for example a sequencing lab to a health care provider or consumer over the network 1830, or alternatively through the mail or a secure download site such as an FTP site.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

TABLE 1
Variants found in highly conserved sequences in non-coding regions
as defined by CTDS. Abbreviations: Chr., Chromoome; Pos., Position
(with reference to GRCh38 38.1/141); Ref., Reference nucleotide;
Alt., Alternative nucleotide

| Chr. | Pos. | Ref | Alt. | Chr. | Pos. | Ref | Alt. |
|---|---|---|---|---|---|---|---|
| 1 | 16996381 | C | T | 3 | 38598916 | A | T |
| 1 | 21884513 | C | T | 3 | 38609965 | C | G |
| 1 | 42930867 | C | G | 3 | 46898193 | G | A |
| 1 | 42930868 | T | G | 3 | 46898193 | G | T |
| 1 | 45013020 | GTAA | G | 3 | 46898660 | A | C |
| 1 | 45013134 | A | G | 3 | 46898660 | A | G |
| 1 | 45332163 | T | G | 3 | 48565083 | C | T |
| 1 | 45332163 | TAC | T | 3 | 48565202 | C | G |
| 1 | 45500414 | G | A | 3 | 48565202 | C | T |
| 1 | 45500415 | T | G | 3 | 48568089 | C | A |
| 1 | 55039507 | C | A | 3 | 48568089 | C | G |
| 1 | 75724821 | A | G | 3 | 48570132 | A | C |
| 1 | 94056830 | C | T | 3 | 48570133 | C | T |
| 1 | 149926919 | C | 1 | 3 | 48570463 | A | G |
| 1 | 150552897 | G | A | 3 | 48581259 | C | A |
| 1 | 154585752 | C | T | 3 | 48581259 | C | T |
| 1 | 154585867 | T | C | 3 | 48584376 | C | G |
| 1 | 155294233 | A | C | 3 | 48584484 | C | A |
| 1 | 155294481 | C | A | 3 | 48584484 | C | G |
| 1 | 155294754 | T | A | 3 | 48584557 | C | T |
| 1 | 155295417 | G | T | 3 | 48587418 | A | C |
| 1 | 155295436 | C | T | 3 | 48587555 | C | T |
| 1 | 155295569 | C | T | 3 | 48588280 | A | G |
| 1 | 155295663 | A | G | 3 | 48588281 | C | T |
| 1 | 155301286 | C | T | 3 | 48591672 | C | G |
| 1 | 156115275 | G | A | 3 | 48591672 | C | T |
| 1 | 156115275 | G | C | 3 | 48592249 | C | G |
| 1 | 156115275 | G | T | 3 | 48592470 | G | T |
| 1 | 160070021 | C | A | 3 | 48592569 | C | T |
| 1 | 161167098 | A | C | 3 | 48592700 | C | A |
| 1 | 161306465 | C | A | 3 | 48592700 | C | G |
| 1 | 161306465 | C | G | 3 | 48592705 | C | T |
| 1 | 161306465 | C | T | 3 | 48592774 | C | T |
| 1 | 161306473 | G | A | 3 | 48593101 | C | G |
| 1 | 161306923 | T | G | 3 | 48593101 | C | T |
| 1 | 173825357 | G | A | 3 | 48593265 | T | G |
| 1 | 193122332 | G | A | 3 | 48593354 | AC | A |
| 1 | 197146140 | C | G | 3 | 48593452 | G | C |
| 1 | 229431720 | C | A | 3 | 48593536 | C | T |
| 1 | 229431903 | C | A | 3 | 48593697 | C | G |
| 1 | 229431993 | CCG | CTT | 3 | 48593699 | G | C |
| 1 | 229432190 | G | T | 3 | 48595346 | G | A |
| 1 | 229432269 | C | T | 3 | 48595347 | G | A |
| 1 | 229432432 | C | T | 3 | 49122703 | C | T |
| 10 | 14953901 | C | A | 3 | 49129836 | T | A |
| 10 | 43105194 | G | A | 3 | 49129838 | C | T |
| 10 | 43114478 | A | G | 3 | 49130730 | TCTCA | T |
| 10 | 72007170 | AGG | ACC | 3 | 49419255 | C | G |
| 10 | 92639901 | G | A | 3 | 49722971 | C | T |
| 10 | 93796966 | A | G | 3 | 49723475 | G | C |
| 10 | 117545628 | G | T | 3 | 52406909 | T | C |
| 10 | 117545631 | G | A | 3 | 52407318 | T | C |
| 10 | 125789036 | A | C | 3 | 52407398 | C | T |
| 11 | 534210 | A | ACCT | 3 | 128483288 | G | A |
| 11 | 819906 | G | A | 3 | 128486802 | C | T |
| 11 | 6390917 | G | T | 3 | 136327256 | GTGAGGACC | G |
| 11 | 17407131 | T | C | 3 | 169765118 | G | C |
| 11 | 17407138 | C | T | 3 | 169765159 | G | C |
| 11 | 17407139 | G | T | 3 | 184170317 | A | G |
| 11 | 17442719 | C | A | 3 | 184170318 | G | A |
| 11 | 17442719 | C | T | 3 | 193593410 | G | A |
| 11 | 17476966 | G | C | 4 | 1002162 | G | A |
| 11 | 17544271 | C | A | 4 | 1002163 | T | C |
| 11 | 31800857 | C | G | 4 | 1002265 | G | A |
| 11 | 31800857 | C | T | 4 | 1004011 | G | C |
| 11 | 31810826 | A | T | 4 | 1004259 | G | A |
| 11 | 32428625 | G | T | 4 | 88075456 | A | G |
| 11 | 32434699 | C | T | 4 | 102869188 | G | A |
| 11 | 46899386 | C | T | 4 | 110621370 | C | A |
| 11 | 47332563 | TA | T | 4 | 110621370 | C | G |
| 11 | 47332564 | A | T | 4 | 177442248 | C | CCCGCAT |
| 11 | 47332565 | C | A | 5 | 1294770 | C | T |
| 11 | 47332565 | C | T | 5 | 1416097 | C | T |
| 11 | 47332703 | C | T | 5 | 36975774 | A | G |
| 11 | 47332704 | T | A | 5 | 36975775 | G | A |
| 11 | 47332705 | G | C | 5 | 41870274 | ACTTTAC | A |
| 11 | 47332705 | G | T | 5 | 90653207 | A | G |
| 11 | 47332813 | C | A | 5 | 132557455 | T | A |
| 11 | 47332813 | C | T | 5 | 149960981 | T | C |
| 11 | 47333189 | C | A | 5 | 150378903 | A | G |
| 11 | 47333189 | C | G | 5 | 150378903 | AG | A |
| 11 | 47333189 | C | T | 5 | 150388089 | G | A |
| 11 | 47333192 | A | C | 5 | 173234749 | C | A |
| 11 | 47333192 | A | G | 5 | 177280562 | CA | C |
| 11 | 47333552 | C | T | 5 | 177402460 | C | T |
| 11 | 47333552 | CGCA | C | 5 | 180620170 | CA | C |
| | | CCAA | | | | | |
| | | CAAC | | | | | |
| | | CT | | | | | |
| 11 | 47333553 | GC | G | 6 | 2948700 | C | T |
| 11 | 47333555 | A | C | 6 | 31860041 | C | G |
| 11 | 47333556 | C | T | 6 | 31860438 | C | T |
| 11 | 47341986 | C | G | 6 | 35512638 | C | T |
| 11 | 47341990 | C | G | 6 | 43045403 | C | G |
| 11 | 47342157 | C | T | 6 | 43576441 | G | C |
| 11 | 47342158 | T | C | 6 | 45422958 | G | A |
| 11 | 47342162 | G | T | 6 | 45422958 | G | C |
| 11 | 47342573 | C | T | 6 | 45546826 | G | C |
| 11 | 47342574 | T | A | 6 | 116877784 | A | G |
| 11 | 47342575 | C | G | 6 | 157174118 | G | C |
| 11 | 47342576 | A | G | 6 | 162727661 | C | A |
| 11 | 47342577 | C | T | 6 | 162727661 | C | T |
| 11 | 47342745 | C | G | 6 | 168441455 | G | T |
| 11 | 47342745 | C | T | 7 | 40134228 | C | T |
| 11 | 47342804 | CCAT | C | 7 | 44145281 | C | A |
| | | GCCC | | | | | |
| | | CGTG | | | | | |
| | | CTTC | | | | | |
| | | TGGA | | | | | |
| | | A | | | | | |
| 11 | 47342828 | A | G | 7 | 44145281 | C | G |
| 11 | 47342936 | C | T | 7 | 44145282 | T | C |
| 11 | 47343019 | A | G | 7 | 44145496 | C | A |
| 11 | 47343020 | C | T | 7 | 44145496 | C | G |
| 11 | 47343158 | C | T | 7 | 44145731 | C | G |
| 11 | 47343264 | T | C | 7 | 44147645 | C | A |
| 11 | 47343281 | C | T | 7 | 44147648 | A | T |
| 11 | 47347030 | C | G | 7 | 44147649 | C | G |
| 11 | 47351507 | T | C | 7 | 44147649 | C | T |
| 11 | 47441822 | C | A | 7 | 44147834 | C | A |
| 11 | 47441923 | T | C | 7 | 44147834 | C | T |
| 11 | 62691423 | T | C | 7 | 44147835 | T | A |
| 11 | 62691424 | G | C | 7 | 44147835 | T | C |
| 11 | 64746809 | C | T | 7 | 44147839 | G | T |
| 11 | 64747221 | C | G | 7 | 66082878 | AG | A |
| 11 | 64754026 | C | A | 7 | 66083175 | G | A |
| 11 | 64755268 | C | T | 7 | 74036585 | G | A |
| 11 | 64755272 | C | G | 7 | 74036585 | G | T |
| 11 | 64755357 | T | A | 7 | 74036586 | T | C |
| 11 | 65532806 | G | A | 7 | 74048502 | G | C |
| 11 | 66849229 | C | T | 7 | 74053160 | C | G |
| 11 | 66870301 | C | T | 7 | 74063229 | G | C |
| 11 | 66871686 | C | A | 7 | 74063309 | G | A |
| 11 | 67519792 | C | A | 7 | 94655985 | C | G |
| 11 | 67611982 | A | C | 7 | 94655989 | C | A |
| 11 | 68039120 | G | C | 7 | 94655989 | C | T |
| 11 | 68039120 | G | T | 7 | 117479846 | G | C |
| 11 | 68043912 | G | A | 7 | 117479869 | G | T |
| 11 | 68043912 | G | C | 7 | 117479930 | G | A |
| 11 | 68049299 | G | A | 7 | 120838776 | C | T |
| 11 | 68049426 | T | C | 7 | 130440932 | A | C |
| 11 | 68049436 | T | A | 7 | 150947880 | T | A |
| 11 | 68049440 | G | A | 7 | 150951442 | A | G |
| 11 | 68049443 | C | T | 7 | 150951447 | C | T |
| 11 | 68049954 | T | C | 7 | 150952424 | C | G |
| 11 | 68049961 | G | A | 7 | 150952424 | C | T |
| 11 | 68050135 | A | C | 7 | 150974709 | A | T |
| 11 | 68050255 | G | A | 7 | 150974710 | C | CCAT |
| 11 | 68050255 | G | T | 7 | 150974942 | C | T |
| 11 | 72195749 | T | C | 7 | 155806295 | C | T |
| 11 | 72195749 | T | G | 7 | 155806576 | C | T |
| 11 | 72240199 | C | T | 7 | 157005875 | T | C |
| 11 | 72243787 | C | T | 7 | 157006478 | C | T |
| 11 | 77147796 | A | G | 7 | 157006478 | CCTGGGT | C |
| 11 | 77190017 | C | G | 8 | 38413915 | T | C |
| 11 | 77190019 | GT | G | 8 | 38414028 | G | T |
| 11 | 112086961 | T | G | 8 | 38414558 | C | T |
| 11 | 118340370 | C | T | 8 | 38418375 | T | C |
| 11 | 119085735 | A | G | 8 | 41797691 | C | T |
| 11 | 119101146 | C | T | 8 | 60781432 | T | A |
| 11 | 119101490 | C | T | 8 | 60781432 | T | C |
| 11 | 124739465 | A | G | 8 | 60844862 | A | G |
| 11 | 124739507 | C | G | 8 | 60844862 | A | T |
| 11 | 124739741 | G | A | 8 | 89984520 | C | T |
| 11 | 130208685 | G | A | 8 | 89984522 | T | TA |
| 12 | 6075330 | C | T | 8 | 89984524 | C | T |
| 12 | 6075333 | A | G | 8 | 118110078 | CACTT | C |
| 12 | 6075334 | C | T | 8 | 118110083 | A | G |
| 12 | 48980693 | A | G | 8 | 118110084 | C | A |
| 12 | 49022152 | C | G | 8 | 118110084 | C | G |
| 12 | 49022279 | C | A | 8 | 118110084 | C | T |
| 12 | 49022279 | C | G | 9 | 6645244 | C | T |
| 12 | 49022355 | T | C | 9 | 34647078 | T | G |
| 12 | 49022589 | C | A | 9 | 34647086 | C | G |
| 12 | 49026181 | C | T | 9 | 34647259 | G | A |
| 12 | 49027324 | T | C | 9 | 34647490 | A | G |
| 12 | 49027325 | G | C | 9 | 35074217 | C | G |
| 12 | 49042880 | T | C | 9 | 35074217 | C | T |
| 12 | 49046425 | C | A | 9 | 35075755 | C | A |
| 12 | 49050885 | C | T | 9 | 35075755 | C | G |
| 12 | 49053206 | C | A | 9 | 35075957 | C | T |
| 12 | 49053322 | C | T | 9 | 35079242 | C | T |
| 12 | 49054306 | C | T | 9 | 35090061 | C | T |
| 12 | 49054419 | T | C | 9 | 37424831 | CCCTTTCCCC | CTT |
| 12 | 49054527 | C | G | 9 | 37424831 | CCCTTTCCCC | CTTT |
| 12 | 49054527 | C | T | 9 | 37424843 | A | G |
| 12 | 49054753 | T | G | 9 | 37430647 | G | A |
| 12 | 51915223 | A | G | 9 | 69035948 | G | A |
| 12 | 51915501 | G | A | 9 | 69035952 | G | C |
| 12 | 51915505 | G | A | 9 | 83971880 | A | AC |
| 12 | 51915505 | G | T | 9 | 95478049 | C | T |
| 12 | 53321870 | G | A | 9 | 97697119 | A | C |
| 12 | 56042170 | G | A | 9 | 126693743 | CA | C |
| 12 | 56042170 | G | C | 9 | 126693745 | G | A |
| 12 | 56042170 | G | T | 9 | 127502773 | G | A |
| 12 | 57628264 | T | C | 9 | 127819661 | C | G |
| 12 | 57765296 | C | T | 9 | 127819661 | C | T |
| 12 | 57766006 | C | T | 9 | 127819662 | T | C |
| 12 | 57766845 | A | C | 9 | 127824975 | C | A |
| 12 | 65171119 | G | A | 9 | 127824975 | C | G |
| 12 | 76348161 | C | A | 9 | 127824976 | T | A |
| 12 | 110281908 | G | C | 9 | 127824977 | A | C |
| 12 | 110340652 | ATTT | A | 9 | 127824981 | G | C |
| | | TAGA | | | | | |
| | | CCAA | | | | | |
| | | TCTG | | | | | |
| | | ACC | | | | | |
| 12 | 114398572 | C | A | 9 | 127825226 | C | G |
| 12 | 114398721 | C | A | 9 | 127825229 | A | G |
| 12 | 114398725 | A | G | 9 | 127825229 | A | T |
| 12 | 120978231 | G | C | 9 | 127825358 | C | T |
| 12 | 120994162 | A | G | 9 | 127825359 | T | A |
| 12 | 120994163 | G | A | 9 | 127825693 | A | G |
| 13 | 32315668 | G | A | 9 | 127825694 | C | A |
| 13 | 48303701 | G | A | 9 | 127825861 | C | G |
| 13 | 48303715 | G | A | 9 | 127825862 | T | C |
| 13 | 48303715 | G | T | 9 | 130479801 | G | A |
| 13 | 48303716 | G | A | 9 | 130479801 | G | GT |
| 13 | 48303720 | T | A | 9 | 130479849 | C | T |
| 13 | 48303720 | T | G | 9 | 132921818 | C | T |
| 13 | 48303721 | G | A | 9 | 136199883 | G | C |
| 13 | 48303724 | G | T | 9 | 136515289 | C | T |
| 13 | 48303763 | G | C | X | 630463 | C | A |
| 13 | 48303764 | G | T | X | 631175 | G | A |
| 13 | 48304050 | G | A | X | 644388 | C | T |
| 13 | 48304050 | G | T | X | 8731829 | C | A |
| 13 | 48304051 | T | G | X | 8731829 | C | G |
| 13 | 50910141 | G | A | X | 13735348 | T | C |
| 13 | 52011779 | C | T | X | 13735349 | A | G |
| 13 | 99983141 | T | A | X | 17721439 | A | G |
| 13 | 113110851 | G | A | X | 17721440 | G | A |
| 13 | 113110851 | G | C | X | 18642157 | C | G |
| 13 | 113110851 | G | T | X | 18642158 | T | C |
| 13 | 113110855 | G | A | X | 18672012 | C | G |
| 13 | 113110855 | G | T | X | 18672014 | T | TA |
| 13 | 113113749 | C | G | X | 18672014 | T | TACCTTCA |
| 13 | 113113750 | A | G | X | 18672015 | A | G |
| 13 | 113148915 | G | A | X | 18672016 | C | A |
| 14 | 24259703 | C | T | X | 18672016 | C | T |
| 14 | 36518021 | C | T | X | 19354461 | G | A |
| 14 | 36518022 | T | A | X | 19354489 | AGGT | A |
| 14 | 36518022 | T | C | X | 20172745 | C | T |
| 14 | 36518022 | T | G | X | 24726579 | A | G |
| 14 | 36518029 | G | T | X | 25010259 | C | G |
| 14 | 36518978 | CACT | C | X | 25015540 | A | G |
| | | T | | | | | |
| 14 | 36518980 | C | G | X | 37727634 | A | G |
| 14 | 36518981 | T | C | X | 37727635 | G | A |
| 14 | 36518984 | C | T | X | 38327335 | C | T |
| 14 | 36662094 | G | T | X | 38327338 | A | C |
| 14 | 49586052 | G | T | X | 38327339 | C | T |
| 14 | 56804187 | C | CCTG | X | 40057322 | C | T |
| 14 | 60648627 | T | A | X | 40062394 | C | A |
| 14 | 73136191 | C | G | X | 40063072 | C | G |
| 14 | 73136796 | G | A | X | 43973299 | C | T |
| 14 | 74241181 | G | A | X | 43973302 | A | C |
| 14 | 93787624 | A | G | X | 43973303 | C | T |
| 14 | 94769660 | C | G | X | 46837203 | G | A |
| 14 | 102928424 | A | G | X | 46837205 | A | G |
| 14 | 102928425 | G | C | X | 46837205 | A | T |
| 14 | 102929087 | G | A | X | 47179165 | T | C |
| 14 | 102930400 | C | T | X | 48509957 | GT | G |
| 14 | 102930503 | C | T | X | 48511936 | G | A |
| 15 | 40405972 | G | T | X | 48512280 | T | A |
| 15 | 43058441 | T | C | X | 48512311 | G | A |
| 15 | 43058442 | T | C | X | 48512325 | G | A |
| 15 | 43105937 | C | G | X | 48512588 | G | C |
| 15 | 43105939 | T | TA | X | 48515715 | A | C |
| 15 | 44711614 | G | T | X | 48515716 | G | C |
| 15 | 72375719 | C | T | X | 48515888 | A | G |
| 15 | 89649739 | A | G | X | 48520373 | AGGGCTACGGC | A |
| | | | | | | ATG | |
| 15 | 96334604 | G | A | X | 48520374 | GGGCTACGGCA | G |
| | | | | | | T | |
| 16 | 2048066 | G | C | X | 48684281 | A | G |
| 16 | 2054295 | G | A | X | 48684282 | G | C |
| 16 | 2054295 | G | T | X | 48684424 | G | A |
| 16 | 2054441 | G | T | X | 48684425 | T | C |
| 16 | 2079429 | G | A | X | 48685545 | A | C |
| 16 | 2079429 | G | C | X | 48685546 | G | A |
| 16 | 2086190 | CAG | C | X | 48685634 | G | A |
| 16 | 2086192 | G | A | X | 48685634 | G | C |
| 16 | 2092479 | C | G | X | 48685634 | G | GT |
| 16 | 2277878 | C | T | X | 48685634 | G | T |
| 16 | 2283456 | G | A | X | 48685636 | A | T |
| 16 | 23641108 | A | C | X | 48688052 | AG | A |
| 16 | 23641109 | C | G | X | 48688052 | AGGCATGTCAG | A |
| | | | | | | CCACGTGGG | |
| 16 | 28482199 | C | A | X | 48688053 | G | A |
| 16 | 28482324 | T | A | X | 48688454 | G | A |
| 16 | 28482472 | C | T | X | 48688455 | T | A |
| 16 | 28936152 | G | T | X | 48688455 | T | C |
| 16 | 30756589 | GATC | G | X | 48688455 | T | G |
| | | T | | | | | |
| 16 | 30980736 | CAG | C | X | 48689067 | G | A |
| 16 | 31464391 | C | A | X | 49075135 | C | T |
| 16 | 31489049 | G | C | X | 49075282 | C | G |
| 16 | 67436156 | C | T | X | 49075360 | TCA | T |
| 16 | 67942609 | A | G | X | 49075362 | A | G |
| 16 | 68645751 | G | A | X | 49075363 | C | T |
| 16 | 68738295 | A | C | X | 49075862 | TCAC | T |
| 16 | 68738295 | A | G | X | 49076429 | C | A |
| 16 | 71570677 | A | C | X | 49076525 | C | T |
| 16 | 74774483 | T | A | X | 49076526 T | G | |
| 17 | 7220198 | G | A | X | 49209761 | T | C |
| 17 | 7223238 | G | A | X | 49210404 | C | T |
| 17 | 7223240 | G | T | X | 49210588 | A | T |
| 17 | 7223629 | A | G | X | 49211482 | C | G |
| 17 | 7223731 | G | A | X | 49217752 | CACTT | C |
| 17 | 8003851 | G | T | X | 49218454 | C | T |
| 17 | 8004157 | G | A | X | 49218881 | C | G |
| 17 | 8015845 | A | T | X | 49218942 | CTG | C |
| 17 | 8110091 | C | A | X | 49230611 | T | C |
| 17 | 15260645 | C | T | X | 49251768 | G | C |
| 17 | 15260647 | CACG | C | X | 49253121 | C | T |
| | | CTG | | | | | |
| 17 | 15260649 | C | A | X | 49253122 | T | C |
| 17 | 15260649 | C | G | X | 49253913 | T | C |
| 17 | 15260649 | C | T | X | 49254068 | C | T |
| 17 | 18143622 | G | A | X | 49255422 | C | G |
| 17 | 18143797 | G | A | X | 49255424 | C | T |
| 17 | 31206221 | G | A | X | 49255425 | T | C |
| 17 | 31206238 | A | C | X | 49255426 | CA | C |
| 17 | 31206238 | A | G | X | 49255510 | C | T |
| 17 | 31206239 | G | T | X | 49255511 | T | G |
| 17 | 31206372 | G | A | X | 53198975 | A | G |
| 17 | 31206372 | G | T | X | 53405492 | C | A |
| 17 | 31206373 | T | A | X | 53413140 | T | C |
| 17 | 35107364 | A | G | X | 53548953 | C | T |
| 17 | 37731832 | T | G | X | 68838616 | G | A |
| 17 | 37731834 | G | C | X | 68839958 | A | G |
| 17 | 41819452 | G | T | X | 68840240 | A | G |
| 17 | 42422626 | C | A | X | 70033397 | G | GATT |
| 17 | 42695516 | TGCA | T | X | 70033529 | G | A |
| 17 | 43104262 | C | G | X | 70033529 | G | T |
| 17 | 43104262 | C | T | X | 70033530 | T | C |
| 17 | 43104262 | CT | C | X | 70033532 | AG | A |
| 17 | 43104263 | T | C | X | 70033533 | G | A |
| 17 | 43104263 | T | G | X | 70033536 | C | A |
| 17 | 43104264 | G | C | X | 70033536 | C | G |
| 17 | 44006527 | A | G | X | 71107922 | C | T |
| 17 | 44006527 | A | T | X | 74422068 | G | A |
| 17 | 44007322 | G | C | X | 74524358 | G | C |
| 17 | 44254490 | TCTC | TTTCAT | X | 77520784 | T | C |
| | | AC | | | | | |
| 17 | 44351362 | G | C | X | 77618986 | G | C |
| 17 | 44351362 | G | T | X | 77618991 | A | T |
| 17 | 44351461 | G | A | X | 78023559 | T | G |
| 17 | 44351797 | T | C | X | 78031398 | A | C |
| 17 | 44352339 | A | G | X | 78031398 | A | G |
| 17 | 44374761 | C | G | X | 78031399 | G | A |
| 17 | 44376303 | C | G | X | 80023047 | C | A |
| 17 | 44380385 | C | T | X | 86047473 | GCTACACAT | GAAGC |
| 17 | 44383489 | T | C | X | 86047479 | C | A |
| 17 | 44384587 | T | A | X | 86047481 | T | TA |
| 17 | 44384587 | T | C | X | 86047483 | C | T |
| 17 | 44385284 | G | T | X | 101348532 | C | A |
| 17 | 44385546 | C | G | X | 101348532 | C | T |
| 17 | 44385550 | C | T | X | 103786463 | A | G |
| 17 | 44385813 | G | T | X | 103786463 | A | T |
| 17 | 44386003 | GACT | G | X | 108440207 | G | C |
| | | C | | | | | |
| 17 | 44386009 | C | T | X | 108440207 | G | T |
| 17 | 50188902 | C | A | X | 108440210 | A | C |
| 17 | 50188902 | C | T | X | 108440210 | A | G |
| 17 | 50189011 | C | T | X | 108559154 | G | A |
| 17 | 50189012 | T | C | X | 108695441 | T | C |
| 17 | 50189164 | T | G | X | 108695442 | A | G |
| 17 | 50189278 | T | C | X | 129553302 | A | G |
| 17 | 50199553 | T | A | X | 136208453 | A | T |
| 17 | 50199554 | A | G | X | 136208642 | G | A |
| 17 | 50199554 | ACC | AGA | X | 149496345 | C | T |
| 17 | 50199555 | C | A | X | 149496518 | T | A |
| 17 | 50199555 | C | T | X | 149496518 | T | C |
| 17 | 50199591 | C | G | X | 149505034 | C | G |
| 17 | 50199591 | C | T | X | 149505034 | CCTGTGGTCGA | C |
| | | | | | | GTTGGCCTGCG | |
| | | | | | | TTTCGGATCCG | |
| | | | | | | AGGGCGACGCA | |
| | | | | | | GACGGAGCTCA | |
| | | | | | | GAACCAGACCC | |
| | | | | | | AGCCAGAGAAG | |
| | | | | | | GCCTCGGCCGG | |
| | | | | | | TCCGGGGTGGC | |
| | | | | | | GGCATTTCGGC | |
| | | | | | | TTCGACGCGGC | |
| | | | | | | CGCTTCAGAGC | |
| | | | | | | GGCGGGGACAG | |
| | | | | | | GCTGCAGCAGG | |
| | | | | | | TGGCGCAGTTA | |
| | | | | | | GCAGCCGCCGC | |
| | | | | | | CGCAGCCACAG | |
| | | | | | | AGACCTCCTCG | |
| | | | | | | TCGGGAACCCA | |
| | | | | | | TGAAGACTGCG | |
| | | | | | | CAACACAGCCG | |
| | | | | | | CCGCCCGGGCC | |
| | | | | | | CGCAGGCCCGG | |
| | | | | | | GCGCTGGCCGC | |
| | | | | | | AGCGCGAGTGC | |
| | | | | | | GTCCGTGCGAC | |
| | | | | | | TCTTCCCTGCGT | |
| | | | | | | CCCTCCCCTCCG | |
| | | | | | | GGGCGGGTTCT | |
| 17 | 50199592 | T | C | X | 153726167 | G | A |
| 17 | 50201410 | C | G | X | 153726167 | G | T |
| 17 | 50201410 | C | T | X | 153729227 | C | A |
| 17 | 58692789 | G | T | X | 153729229 | C | G |
| 17 | 61398941 | G | C | X | 153729230 | A | C |
| 17 | 72122717 | A | C | X | 153729231 | G | A |
| 17 | 72122717 | A | G | X | 153736256 | T | C |
| 17 | 72122973 | G | A | X | 153736256 | T | G |
| 17 | 75727507 | T | A | X | 153736343 | A | C |
| 17 | 80108829 | G | A | X | 153736343 | A | G |
| 18 | 22176953 | A | G | X | 153736514 | G | A |
| 18 | 57586548 | A | C | X | 153737155 | A | G |
| 18 | 57586549 | C | T | X | 153737252 | G | A |
| 18 | 57586551 | T | G | X | 153863550 | C | G |
| 18 | 57586552 | A | C | X | 153863550 | C | T |
| 18 | 57586553 | C | A | X | 153864019 | T | C |
| 18 | 57586553 | C | G | X | 153864320 | A | G |
| 18 | 57586553 | C | T | X | 153864583 | A | T |
| 18 | 57586871 | C | G | X | 153864584 | CCT | C |
| 18 | 79988603 | CGCG | C | X | 153864705 | C | T |
| | | CGCG | | | | | |
| | | CTAG | | | | | |
| | | CGCC | | | | | |
| | | GTGC | | | | | |
| | | GTGC | | | | | |
| | | TGAC | | | | | |
| | | GGCA | | | | | |
| | | TGT | | | | | |
| 19 | 855795 | G | A | X | 153865087 | C | T |
| 19 | 855795 | G | C | X | 153865838 | T | G |
| 19 | 855795 | G | T | X | 153867554 | C | T |
| 19 | 855797 | A | T | X | 153867795 | C | T |
| 19 | 855799 | G | A | X | 153867799 | C | T |
| 19 | 855799 | G | T | X | 153867911 | C | G |
| 19 | 920280 | AC | A | X | 153868123 | C | T |
| 19 | 1207204 | G | A | X | 153868197 | C | A |
| 19 | 1207204 | G | T | X | 153868460 | T | A |
| 19 | 1207205 | T | A | X | 153868559 | A | G |
| 19 | 1220367 | CCGC | CTGCA | X | 153868559 | A | T |
| | | AGG | C | | | | |
| 19 | 1220369 | G | A | X | 153868836 | C | T |
| 19 | 1220371 | A | G | X | 153868953 | C | T |
| 19 | 1220371 | AGG | AC | X | 153868954 | T | A |
| 19 | 1220372 | G | A | X | 153869664 | C | G |
| 19 | 1220506 | G | A | X | 153869664 | C | T |
| 19 | 1220506 | G | T | X | 153869802 | C | T |
| 19 | 1220507 | T | A | X | 153870785 | C | T |
| 19 | 1220579 | A | T | X | 153870961 | C | T |
| 19 | 1220718 | G | A | X | 153870962 | T | G |
| 19 | 1220718 | G | GT | X | 153871045 | G | A |
| 19 | 1220718 | G | T | X | 153871052 | C | T |
| 19 | 1220719 | T | C | X | 153872587 | C | G |
| 19 | 1220722 | G | A | X | 153872591 | C | T |
| 19 | 2250761 | G | A | X | 153872698 | C | T |
| 19 | 3586494 | G | T | X | 153872699 | T | C |
| 19 | 3586681 | G | A | X | 153971810 | A | G |
| 19 | 6712507 | C | A | X | 154092175 | GTTAC | G |
| 19 | 6712625 | T | A | X | 154351698 | T | C |
| 19 | 7550431 | T | G | X | 154359234 | CCACCTCCT | C |
| 19 | 11021968 | G | C | X | 154359244 | A | C |
| 19 | 11105217 | C | T | X | 154361788 | C | T |
| 19 | 11105218 | A | C | X | 154362416 | A | C |
| 19 | 11105219 | G | A | X | 154362417 | C | G |
| 19 | 11105219 | G | GC | X | 154364525 | C | T |
| 19 | 11106688 | G | A | X | 154364721 | T | C |
| 19 | 11106688 | G | T | X | 154364819 | A | C |
| 19 | 11106689 | T | C | X | 154364959 | T | C |
| 19 | 11107389 | C | G | X | 154365487 | C | A |
| 19 | 11107390 | A | G | X | 154370872 | C | T |
| 19 | 11107391 | G | A | X | 154379567 | G | T |
| 19 | 11107391 | GTGA | G | X | 154379571 | G | C |
| | | CACT | | | | | |
| | | C | | | | | |
| 19 | 11129671 | G | A | X | 154379795 | G | A |
| 19 | 12648404 | T | C | X | 154379795 | G | T |
| 19 | 12656947 | A | C | X | 154380231 | A | G |
| 19 | 12656947 | A | T | X | 154380232 | A | G |
| 19 | 12656948 | C | G | X | 154380233 | G | C |
| 19 | 12806801 | G | C | X | 154412216 | T | G |
| 19 | 12887264 | A | G | X | 154419541 | A | G |
| 19 | 12887294 | G | A | X | 154419624 | G | A |
| 19 | 12891400 | G | T | X | 154419624 | G | T |
| 19 | 12891829 | A | T | X | 154419697 | CTCACCAGGGA | C |
| | | | | | | AAG | |
| 19 | 12896426 | G | A | X | 154419748 | T | C |
| 19 | 12938404 | C | T | X | 154419751 | G | A |
| 19 | 12938561 | G | C | X | 154420265 | G | T |
| 19 | 15192300 | T | C | X | 154420656 | A | C |
| 19 | 18599564 | C | T | X | 154420657 | G | A |
| 19 | 34399554 | A | C | X | 154420657 | G | GA |
| 19 | 35844099 | TCA | T | X | 154420736 | G | A |
| 19 | 35844249 | G | C | X | 154420737 | A | T |
| 19 | 35844317 | A | G | X | 154420901 | A | G |
| 19 | 35846006 | A | C | X | 154420902 | G | T |
| 19 | 40605654 | T | G | X | 154532464 | T | C |
| 19 | 45363914 | G | T | X | 154534034 | CCG | CAT |
| 19 | 49862180 | C | CTT | X | 154547746 | G | T |
| 2 | 3575685 | G | A | X | 154765429 | T | C |
| 2 | 3575889 | G | A | X | 154765439 | C | G |
| 2 | 3575889 | G | T | X | 154863076 | CACTT | C |
| 2 | 11785078 | T | C | X | 154863078 | C | G |
| 2 | 26263480 | G | C | X | 154863080 | T | G |
| 2 | 26263483 | A | T | X | 154863082 | C | A |
| 2 | 26473569 | T | G | X | 154863082 | C | G |
| 2 | 26483461 | C | A | X | 154863082 | C | T |
| 2 | 27312679 | C | A | X | 154863228 | C | G |
| 2 | 27312992 | A | G | X | 154863229 | T | C |
| 2 | 27312993 | C | A | X | 154863230 | G | C |
| 2 | 32064247 | G | A | X | 154863234 | GGAGAGATTA | G |
| 2 | 32064247 | G | T | X | 154863241 | T | C |
| 2 | 32127023 | G | A | X | 154901369 | AC | A |
| 2 | 47403403 | G | C | X | 154901370 | C | T |
| 2 | 61853858 | C | A | X | 154904525 | C | T |
| 2 | 73927053 | G | A | X | 154904526 | T | A |
| 2 | 96293315 | C | A | X | 154904526 | T | C |
| 2 | 127422915 | A | T | X | 154904617 | G | A |
| 2 | 127422942 | G | A | X | 154906414 | A | C |
| 2 | 127422947 | T | G | X | 154906418 | A | C |
| 2 | 127423006 | T | G | X | 154906419 | C | A |
| 2 | 127423030 | G | A | X | 154906419 | C | G |
| 2 | 127423033 | G | A | X | 154906419 | C | T |
| 2 | 127423409 | GTGA | G | X | 154928568 | T | C |
| | | GA | | | | | |
| 2 | 151524617 | C | T | X | 154928568 | T | G |
| 2 | 171435079 | TTAG | TAA | X | 154928569 | A | C |
| 2 | 176093672 | G | A | X | 154928569 | A | G |
| 2 | 178553911 | TACC | T | X | 154928570 | C | A |
| 2 | 202377551 | G | T | X | 154928570 | C | T |
| 2 | 202377552 | T | C | X | 155264073 | C | T |
| 2 | 218661308 | G | T | Y | 2787733 | C | G |
| 20 | 968139 | C | T | | | | |
| 20 | 3082975 | AC | A | | | | |
| 20 | 3229016 | AGCA | ACCGG | | | | |
| | | GACG | CCGGC | | | | |
| | | GGCA | C | | | | |
| 20 | 3229094 | C | T | | | | |
| 20 | 3889730 | T | G | | | | |
| 20 | 8132751 | G | A | | | | |
| 20 | 10639957 | T | C | | | | |
| 20 | 10641245 | C | G | | | | |
| 20 | 10641246 | T | C | | | | |
| 20 | 10641251 | CGAT | C | | | | |
| | | TTT | | | | | |
| 20 | 18057908 | A | C | | | | |
| 20 | 18057941 | A | G | | | | |
| 20 | 18058004 | A | G | | | | |
| 20 | 18507416 | A | G | | | | |
| 20 | 21708712 | G | A | | | | |
| 20 | 23049806 | G | T | | | | |
| 20 | 34955722 | C | T | | | | |
| 20 | 46709745 | G | A | | | | |
| 20 | 49936342 | C | A | | | | |
| 20 | 49936344 | C | A | | | | |
| 20 | 58909948 | TA | T | | | | |
| 20 | 58909949 | A | G | | | | |
| 21 | 26171136 | G | C | | | | |
| 21 | 26171301 | C | T | | | | |
| 21 | 34886842 | C | A | | | | |
| 21 | 34886842 | C | T | | | | |
| 22 | 19755950 | C | T | | | | |
| 22 | 19756055 | C | T | | | | |
| 22 | 19756212 | A | C | | | | |
| 22 | 20431017 | C | A | | | | |
| 22 | 20994728 | GT | G | | | | |
| 22 | 29604113 | G | A | | | | |
| 22 | 29604114 | T | C | | | | |
| 22 | 29674835 | G | A | | | | |
| 22 | 36284091 | A | T | | | | |
| 22 | 41515536 | G | T | | | | |
| 22 | 50526241 | C | T | | | | |
| 22 | 50526244 | A | T | | | | |
| 22 | 50526478 | TGCG | T | | | | |
| | | G | | | | | |
| 22 | 50526575 | C | T | | | | |
| 22 | 50529339 | C | G | | | | |
| 3 | 10142188 | G | A | | | | |
| 3 | 10142188 | G | C | | | | |
| 3 | 10142188 | G | T | | | | |
| 3 | 10142189 | TACG | TCG | | | | |
| | | GGCC | | | | | |
| | | C | | | | | |
| 3 | 10142194 | G | A | | | | |
| 3 | 33097008 | T | TA | | | | |
| 3 | 33097009 | ACGC | A | | | | |
| | | GCAA | | | | | |
| | | GCCG | | | | | |
| 3 | 33097010 | C | G | | | | |
| 3 | 33114549 | G | C | | | | |
| 3 | 33114550 | C | A | | | | |
| 3 | 33114550 | C | G | | | | |
| 3 | 36993664 | G | A | | | | |
| 3 | 36993668 | G | C | | | | |

TABLE 2
SNPs located in non-coding regions that are highly conserved by CDTS as annotated by rs number.

rs587780751; rs745366624; rs777251123; rs778796405; rs774531501; rs587776927; rs768823171;
rs749303140; rs376829288; rs750530042; rs587776558; rs372686280; rs111812550; rs143144732;
rs193922699; rs750180293; rs398122808; rs757171524; rs773306994; rs773306994; rs372418954;
rs762425885; rs397516031; rs397516022; rs730880592; rs730880592; rs397516020; rs397516020;
rs373746463; rs373746463; rs373746463; rs387906397; rs387906397; rs587782958; rs730880718;
rs730880667; rs113358486; rs111683277; rs112917345; rs730880691; rs397515916; rs730880690;
rs111437311; rs397515903; rs727503201; rs112999777; rs397515897; rs727503204; rs397515893;
rs397515891; rs587776699; rs587776700; rs376395543; rs748486465; rs149712664; rs199683937;
rs144637717; rs587776644; rs730880296; rs397515322; rs558721552; rs531105836; rs587777262;
rs267607302; rs387907354; rs398123750; rs727503988; rs587783714; rs148622862; rs763991428;
rs761780097; rs770204470; rs387906521; rs387906520; rs79367981; rs749160734; rs587776708;
rs587776708; rs34086577; rs199959804; rs587777290; rs386834170; rs386834169; rs144077391;
rs386834164; rs386834166; rs770093080; rs587777374; rs45517105; rs45517105; rs45488500;
rs45517289; rs45517289; rs137854118; rs45517358; rs189077405; rs515726118; rs386833742;
rs386833739; rs755127868; rs200655247; rs376023420; rs747351687; rs113690956; rs376281637;
rs765390290; rs773401248; rs61750189; rs530975087; rs201978571; rs267604791; rs80358116;
rs80358116; rs273899695; rs80358011; rs80358011; rs80358051; rs730880267; rs63751296; rs63750707;
rs776442328; rs776820510; rs72653165; rs72667012; rs72667008; rs527398797; rs587780009;
rs587776658; rs587782018; rs745620135; rs372651309; rs556992558; rs137853932; rs200253809;
rs386833901; rs770882876; rs750550558; rs397507554; rs730880306; rs201613240; rs147952488;
rs770241629; rs373494631; rs397517741; rs386833856; rs559854357; rs371496308; rs539645405;
rs187510057; rs41298629; rs536892777; rs747330606; rs748559929; rs770277446; rs201685922;
rs767245071; rs730882032; rs587776525; rs398123358; rs72659359; rs137853943; rs267607709;
rs267607710; rs766168993; rs775288140; rs780041521; rs145564018; rs775456047; rs587776879;
rs540289812; rs745832717; rs745915863; rs386833418; rs199422309; rs431905514; rs587784059;
rs748086984; rs386833492; rs199988476; rs281865166; rs587776515; rs397518439; rs193922258;
rs142637046; rs73717525; rs145483167; rs587777285; rs747737281; rs183894680; rs116735828;
rs574673404; rs386833563; rs768154316; rs111033661; rs755363896; rs368953604; rs180177319;
rs148049120; rs150676454; rs372655486; rs373842615; rs763389916; rs118203419; rs515726232;
rs312262809; rs312262804; rs281865349; rs281865338; rs281865337; rs281865334; rs281865336;
rs281865336; rs62638626; rs62638627; rs587784423; rs113951193; rs281874765; rs104886349;
rs398123247; rs74315277; rs200346587; rs398122908; rs727503036; rs3975155747; rs587776734

TABLE 3
CDTS 1^{st} percentile specific variants (most conserved), Abbreviations. Chr.,
Chromosome; Pos., Position (with reference to GRCh38 38.1/141); Ref.,
Reference nucleotide; Alt., Alernative nucleotide.

| Chr. | Pos. | Ref. | Alt. | Chr. | Pos. | Ref | Alt. |
|---|---|---|---|---|---|---|---|
| 1 | 21884513 | C | T | 5 | 138947482 | C | T |
| 1 | 45331862 | A | C | 5 | 138947491 | C | T |
| 1 | 55039507 | C | A | 5 | 173245288 | C | T |
| 1 | 155293394 | G | A | 5 | 173245300 | C | G |
| 1 | 155293395 | A | G | 6 | 42966120 | G | C |
| 1 | 155295417 | G | T | 6 | 42966214 | C | T |
| 1 | 155301286 | C | T | 6 | 43042773 | C | T |
| 1 | 173853326 | ATGTTTAC | A | 6 | 116877784 | A | G |
| | | TCTTC | | | | | |
| 10 | 49473613 | T | C | 7 | 117479869 | G | T |
| 10 | 87958026 | A | G | 7 | 117479930 | G | A |
| 10 | 125789036 | A | C | 7 | 155806576 | C | T |
| 11 | 17407138 | C | T | 7 | 156268812 | C | T |
| 11 | 17407139 | G | T | 8 | 41797691 | C | T |
| 11 | 17476966 | G | C | 8 | 60862343 | G | T |
| 11 | 47342162 | G | T | 8 | 118110078 | CACTT | C |
| 11 | 47342804 | CCATGCCC | C | 9 | 21968347 | T | C |
| | | GTGCTTCTC | | | | | |
| | | AA | | | | | |
| 11 | 47343158 | C | T | 9 | 34647078 | T | G |
| 11 | 47343281 | C | T | 9 | 37424831 | CCCTTTCC | CTT |
| | | | | | | C | |
| 11 | 47346379 | C | T | 9 | 37424831 | CCCTTTCC | CTTT |
| | | | | | C | | |
| 11 | 47346380 | G | T | 9 | 127824981 | G | C |
| 11 | 47347065 | C | T | 9 | 127826683 | A | T |
| 11 | 47347489 | G | C | 9 | 128522658 | A | G |
| 11 | 57614315 | G | A | 9 | 130479849 | C | T |
| 11 | 64804825 | G | C | X | 8568225 | CACTT | C |
| 11 | 64805019 | GCAGCTGT | G | X | 9743804 | A | C |
| | | CCT | | | | | |
| 11 | 64805019 | GCAGCTGT | GAT | X | 13735238 | T | A |
| | | CCTCAC | | | | | |
| 11 | 64807228 | C | T | X | 19354461 | G | A |
| 11 | 66526640 | C | G | X | 20173150 | A | C |
| 11 | 68049426 | T | C | X | 20195156 | T | C |
| 11 | 68049436 | T | A | X | 24726579 | A | G |
| 11 | 68049440 | G | A | X | 31209490 | ATACGTAC | AAT |
| 11 | 68049443 | C | T | X | 31444636 | A | T |
| 11 | 68049954 | T | C | X | 37782077 | T | G |
| 11 | 119084613 | G | A | X | 48512280 | T | A |
| 11 | 119084703 | C | T | X | 48512311 | G | A |
| 11 | 119084764 | G | A | X | 48685939 | T | G |
| 11 | 119085735 | A | G | X | 49255422 | C | G |
| 11 | 119101146 | C | T | X | 50081633 | A | G |
| 11 | 124739465 | A | G | X | 73852757 | G | C |
| 11 | 124739741 | G | A | X | 77618991 | A | T |
| 12 | 53425642 | C | T | X | 78011443 | T | TATAAG |
| 12 | 56092977 | A | G | X | 78023559 | T | G |
| 12 | 65963237 | TGTTCCAG | T | X | 80023047 | C | A |
| 12 | 88068657 | A | T | X | 85900715 | A | C |
| 12 | 110339493 | A | G | X | 86047473 | GCTACACC | GAAGC |
| 12 | 120978231 | G | C | X | 101354702 | GCAAA | G |
| 12 | 120978307 | G | A | X | 101354717 | T | C |
| 12 | 120999262 | G | A | X | 101358705 | AC | AAGTTTT |
| | | | | | | | CCCCT |
| 13 | 52011547 | T | A | X | 101358707 | G | T |
| 13 | 113118403 | T | C | X | 108570694 | G | A |
| 14 | 73136191 | C | G | X | 108595489 | T | A |
| 14 | 73136796 | G | A | X | 108601867 | A | G |
| 14 | 102929087 | G | A | X | 108695042 | T | C |
| 14 | 102930400 | C | T | X | 120470223 | T | G |
| 14 | 102930503 | C | T | X | 129553302 | A | G |
| 15 | 43038157 | G | C | X | 134377997 | A | G |
| 15 | 43058441 | T | C | X | 134491434 | A | G |
| 15 | 43058442 | T | C | X | 134494792 | T | A |
| 16 | 1362442 | G | A | X | 139548353 | CTTCT | C |
| 16 | 2093103 | G | T | X | 139548354 | T | G |
| 16 | 2283456 | G | A | X | 139548355 | T | G |
| 16 | 2308643 | C | T | X | 139548504 | A | G |
| 16 | 28486663 | C | G | X | 150649703 | T | A |
| 16 | 50779517 | A | G | X | 153865838 | T | G |
| 16 | 67436156 | C | T | X | 153868197 | C | A |
| 16 | 83914942 | A | G | X | 153871045 | G | A |
| 17 | 1400568 | C | A | X | 154359234 | CCACCTCC | C |
| 17 | 3648823 | T | C | X | 154765429 | T | C |
| 17 | 7223629 | A | G | X | 154863234 | GGAGAGA | G |
| | | | | | | TA | |
| 17 | 31161118 | T | G | X | 154863241 | T | C |
| 17 | 31206221 | G | A | X | 154902965 | C | T |
| 17 | 31334559 | A | G | X | 154904122 | T | A |
| 17 | 31337600 | A | G | X | 154904617 | G | A |
| 17 | 41819452 | G | T | X | 154931683 | ATGAGGA | A |
| | | | | | | GAATAAGA | |
| | | | | | | CTC | |
| 17 | 44386003 | GACTC | G | X | 154947686 | A | T |
| 17 | 50189549 | A | C | X | 154961183 | G | T |
| 17 | 50194840 | C | T | X | 154969566 | A | C |
| 17 | 50199462 | T | C | X | 154987311 | A | G |
| 17 | 61398941 | G | C | X | 154987316 | A | C |
| 17 | 80108689 | G | A | X | 154987337 | T | C |
| 18 | 22181443 | C | G | X | 154991304 | C | T |
| 18 | 51078250 | T | C | X | 154999606 | T | C |
| 18 | 57586871 | C | G | X | 154999611 | A | C |
| 18 | 79988603 | CGCGCGCC | C | X | 154999626 | T | A |
| | | TAGCGCCG | | | | | |
| | | GCGTGCTG | | | | | |
| | | CGGCATGT | | | | | |
| 19 | 855556 | C | A | Y | 2787733 | C | G |
| 19 | 920280 | AC | A | | | | |
| 19 | 1220367 | CCGCAGG | CTGCAC | | | | |
| 19 | 1399509 | C | A | | | | |
| 19 | 12887294 | G | A | | | | |
| 19 | 35844249 | G | C | | | | |
| 19 | 38523211 | C | G | | | | |
| 19 | 45364557 | C | T | | | | |
| 2 | 69245213 | A | G | | | | |
| 2 | 97733464 | G | A | | | | |
| 2 | 108930249 | ACAAAGGC | A | | | | |
| | | GGTGTTGTT | | | | | |
| | | G | | | | | |
| 2 | 127423006 | T | G | | | | |
| 2 | 227303992 | A | G | | | | |
| 20 | 10641251 | CGATTTT | C | | | | |
| 20 | 18507416 | A | G | | | | |
| 20 | 18546201 | A | G | | | | |
| 20 | 21708712 | G | A | | | | |
| 20 | 21709456 | A | C | | | | |
| 20 | 49936342 | C | A | | | | |
| 20 | 49936344 | C | A | | | | |
| 20 | 63408542 | G | T | | | | |
| 22 | 19755950 | C | T | | | | |
| 22 | 19756055 | C | T | | | | |
| 22 | 19756212 | A | C | | | | |
| 3 | 10142194 | G | A | | | | |
| 3 | 46858471 | G | A | | | | |
| 3 | 48565083 | C | T | | | | |
| 3 | 48575248 | A | C | | | | |
| 3 | 48576781 | A | C | | | | |
| 3 | 48592705 | C | T | | | | |
| 3 | 122275793 | A | C | | | | |
| 4 | 1001672 | G | A | | | | |
| 4 | 42963153 | A | T | | | | |
| 4 | 110618699 | T | C | | | | |

TABLE 4
SNPs located in CDTS 1^{st} percentile non-coding regions that
are highly conserved by CDTS as annotated by rs number.

rs778796405; rs8177982; rs376829288; rs4253196; rs750180293; rs757171524; rs727503201;
rs397515893; rs587776699; rs397516083; rs201078659; rs750425291; rs558721552; rs531105836;
rs200782636; rs752197734; rs3093266; rs34086577; rs199959804; rs144077391; rs386834164;
rs386834166; rs189077405; rs746701685; rs386833721; rs376023420; rs761146008; rs765390290;
rs72648337; rs527398797; rs367567416; rs372651309; rs200253809; rs193922837; rs761737358;
rs113994173; rs559854357; rs111951711; rs371496308; rs368123079; rs118192239; rs41298629;
rs536892777

What is claimed is:

1. A functional genomic assay comprising:
a) identifying a presence of at least one genomic sequence variant in a nucleic acid sequence of an individual; and
b) determining if the at least one genomic sequence variant occurs in a highly conserved genomic region,
the highly conserved genomic region having an observed context dependent tolerance score greater than an expected context dependent tolerance score, wherein the expected context dependent tolerance score is the probability to vary of a unique nucleic acid sequence of n-nucleotides in length in a certain region of x nucleotides in length in a plurality of genomes, and the observed context dependent tolerance score is a number of genomic sequence variants in the certain region of x nucleotides in length actually observed in the plurality of genomes.

2. The functional genomic assay of claim 1, wherein the nucleic acid sequence comprises a DNA sequence.

3. The functional genomic assay of claim 2, wherein the DNA sequence comprises a nuclear DNA sequence.

4. The functional genomic assay of claim 1, wherein the plurality of genomes is at least 10,000 genomes.

5. The functional genomic assay of claim 1, wherein the nucleic acid sequence comprises at least 100,000 nucleotides.

6. The functional genomic assay of claim 1, comprising identifying the presence of at least 10 genomic sequence variants.

7. The functional genomic assay of claim 1, wherein the at least one genomic sequence variant comprises at least one of an insertion, a deletion, and a translocation.

8. The functional genomic assay of claim 1, wherein the at least one genomic sequence variant comprises a single nucleotide polymorphism.

9. The functional genomic assay of claim 1, wherein n equals 7.

10. The functional genomic assay of claim 1, wherein x is between 400 and 600.

11. The functional genomic assay of claim 1, comprising determining if the at least one genomic sequence variant is in a non-coding highly conserved genomic region.

12. The functional genomic assay of claim 11, the at least one genomic sequence variant is in a non-coding highly conserved genomic region within 2 megabases of a known disease-associated gene.

13. The functional genomic assay of claim 1, wherein the highly conserved genomic region is a genomic region corresponding to a most conserved 1^{st} percentile of all genomic regions.

14. The functional genomic assay of claim 1, wherein the observed context dependent tolerance score is at least 10% greater than an expected context dependent tolerance score.

15. The functional genomic assay of claim 1, wherein at least one of the at least one genomic sequence variant in a highly conserved genomic region is selected from the list consisting of rs587780751, rs745366624, rs777251123, rs778796405, rs774531501, rs587776927, rs768823171, rs749303140, rs376829288, rs750530042, rs587776558, rs372686280, rs111812550, rs143144732, rs193922699, rs750180293, rs398122808, rs757171524, rs773306994, rs773306994, rs372418954, rs762425885, rs397516031, rs397516022, rs730880592, rs730880592, rs397516020, rs397516020, rs373746463, rs373746463, rs373746463, rs387906397, rs387906397, rs587782958, rs730880718, rs730880667, rs113358486, rs111683277, rs112917345, rs730880691, rs397515916, rs730880690, rs111437311, rs397515903, rs727503201, rs112999777, rs397515897, rs727503204, rs397515893, rs397515891, rs587776699, rs587776700, rs376395543, rs748486465, rs149712664, rs199683937, rs144637717, rs587776644, rs730880296, rs397515322, rs558721552, rs531105836, rs587777262, rs267607302, rs387907354, rs398123750, rs727503988, rs587783714, rs148622862, rs763991428, rs761780097, rs770204470, rs387906521, rs387906520, rs79367981, rs749160734, rs587776708, rs587776708, rs34086577, rs199959804, rs587777290, rs386834170, rs386834169, rs144077391, rs386834164, rs386834166, rs770093080, rs587777374, rs45517105, rs45517105, rs45488500, rs45517289, rs45517289, rs137854118, rs45517358, rs189077405, rs515726118, rs386833742, rs386833739, rs755127868, rs200655247, rs376023420, rs747351687, rs113690956, rs376281637, rs765390290, rs773401248, rs61750189, rs530975087, rs201978571, rs267604791, rs80358116, rs80358116, rs273899695, rs80358011, rs80358011, rs80358051, rs730880267, rs63751296, rs63750707, rs776442328, rs776820510, rs72653165, rs72667012, rs72667008, rs527398797, rs587780009, rs587776658, rs587782018, rs745620135, rs372651309, rs556992558, rs137853932, rs200253809, rs386833901, rs770882876, rs750550558, rs397507554, rs730880306, rs201613240, rs147952488, rs770241629, rs373494631, rs397517741, rs386833856, rs559854357, rs371496308, rs539645405, rs187510057, rs41298629, rs536892777, rs747330606, rs748559929, rs770277446, rs201685922, rs767245071, rs730882032, rs587776525, rs398123358, rs72659359, rs137853943, rs267607709, rs267607710, rs766168993, rs775288140, rs780041521, rs145564018, rs775456047, rs587776879, rs540289812, rs745832717, rs745915863, rs386833418, rs199422309, rs431905514, rs587784059, rs748086984, rs386833492, rs199988476, rs281865166, rs587776515, rs397518439, rs193922258, rs142637046, rs73717525, rs145483167, rs587777285, rs747737281, rs183894680, rs116735828, rs574673404, rs386833563, rs768154316, rs111033661, rs755363896, rs368953604, rs180177319, rs148049120, rs150676454, rs372655486, rs373842615, rs763389916, rs118203419, rs515726232, rs312262809, rs312262804, rs281865349, rs281865338, rs281865337, rs281865334, rs281865336, rs281865336, rs62638626, rs62638627, rs587784423, rs113951193, rs281874765, rs104886349, rs398123247, rs74315277, rs200346587, rs398122908, rs727503036, rs397515747, and rs587776734.

16. The functional genomic assay of claim 1, wherein at least one of the at least one genomic sequence variant in a highly conserved genomic region is selected from the list consisting of rs778796405, rs8177982, rs376829288, rs4253196, rs750180293, rs757171524, rs727503201, rs397515893, rs587776699, rs397516083, rs201078659, rs750425291, rs558721552, rs531105836, rs200782636, rs752197734, rs3093266, rs34086577, rs199959804, rs144077391, rs386834164, rs386834166, rs189077405, rs746701685, rs386833721, rs376023420, rs761146008, rs765390290, rs72648337, rs527398797, rs367567416; rs372651309, rs200253809, rs193922837, rs761737358, rs113994173, rs559854357, rs111951711, rs371496308, rs368123079, rs118192239, rs41298629, and rs536892777.

17. The functional genomic assay of claim 1 for use in determining a likelihood of the individual being diagnosed with a cancer.

18. The functional genomic assay of claim 1 for use in prognosing a cancer of the individual.

19. The functional genomic assay of claim 1 for use in determining a longevity of the individual.

20. A method of identifying a relative genomic health risk of a genomic sequence variant in a DNA sequence of an individual, the method comprising:
a) determining at least one genomic sequence variant in the DNA sequence of the individual; wherein the genomic sequence variant is a difference of at least one nucleotide in the individual when compared to a corresponding position in a reference genome; and
b) comparing the at least one genomic sequence variant of the individual to a tolerability score at a corresponding position within x nucleotides of a genetic element, wherein the tolerability score comprises a function of a nucleotide variation score and an allele proportion score, wherein the nucleotide variation score is the variance observed in a plurality of genomes at the corresponding position, and the allele proportion score is the proportion of genomic variants that exceeds an incidence of 0.0001 in the plurality of genomes at the corresponding position.

21. A method of identifying a relative genomic health risk of a genomic sequence variant in a DNA sequence of an individual, the method comprising:
a) determining at least one genomic sequence variant in the DNA sequence of the individual; wherein the genomic sequence variant is a difference of at least one nucleotide in the individual when compared to a corresponding position in a reference genome; and
b) determining an n-variant score for the at least one genomic sequence variant, wherein the n-variant score comprises a function of a count score and an allele frequency score, wherein the count score is the ratio of the number of times any genomic sequence variant occurs in a unique sequence of n-nucleotides in length in the plurality of genomes to the number of times that the unique sequence of n-nucleotides in length occurs in the reference genome, and the allele frequency score is the frequency of the proportion of genomic sequence variants that are fixed in the population, at an allele frequency greater than 0.0001 in the plurality of genomes.

22. A method of identifying a relative genomic health risk of a genomic sequence variant of an individual, the method comprising:
a) determining at least one genomic sequence variant in a DNA sequence of the individual; wherein the genomic sequence variant is a difference of at least one nucleotide in the individual when compared to a corresponding position in a reference genome; and
b) determining if the at least one genomic sequence variant occurs within a region with a low context dependent tolerance score, wherein the context dependent tolerance score comprises a function of an observed context dependent tolerance score and an expected context dependent tolerance score, wherein the expected context dependent tolerance score is the overall probability to vary of a unique sequence of n-nucleotides in length in a certain region of x nucleotides in length in a plurality of genomes, and the observed context dependent tolerance score is a number of genomic sequence variants in a certain region of x nucleotides in length actually observed and fixed in the plurality of genomes as a function of a length of the region.

23. A method of identifying a relative genomic health risk of a genomic sequence variant of an individual, the method comprising:
a) determining at least one genomic sequence variant in a DNA sequence of the individual; wherein the genomic sequence variant is a difference of at least one nucleotide in the individual when compared to a corresponding position in a reference genome;
b) determining if the at least one genomic sequence variant causes an amino acid variant in an expressed protein, wherein the amino acid variant is a difference of at least one amino acid when compared to a reference genome; and
c) comparing the amino acid variant to a protein tolerability score at a corresponding position within a defined protein class, wherein the protein tolerability score comprises a diversity score, missense score, and a protein allele frequency score, wherein the diversity score is a normalized diversity metric, the missense score is the variance observed in a plurality of genomes at the corresponding position which leads to an amino acid mutation, and the protein allele frequency score is the proportion of genomic variants that leads to an amino acid variant that exceeds an incidence of 0.0001 in the plurality of genomes at the corresponding position.

24. A computer-implemented system comprising: a computer comprising: at least one processor, a memory, an operating system configured to perform executable instructions, and a computer program including instructions executable by the at least one processor to create a functional genomic assay application, the functional genomic assay application configured to perform the following:
a) receiving a nucleic acid sequence of an individual;
b) identifying a presence of at least one genomic sequence variant in the nucleic acid sequence of the individual; and
c) determining if the at least one genomic sequence variant occurs in a highly conserved genomic region,
the highly conserved genomic region having an observed context dependent tolerance score greater than an expected context dependent tolerance score, wherein the expected context dependent tolerance score is the probability to vary of a unique nucleic acid sequence of n-nucleotides in length in a certain region of x nucleotides in length in a plurality of genomes, and the observed context dependent tolerance score is a number of genomic sequence variants in the certain region of x nucleotides in length actually observed in the plurality of genomes.

25. The computer-implemented system of claim 24, wherein the nucleic acid sequence comprises a DNA sequence.

26. The computer-implemented system of claim 25, wherein the DNA sequence comprises a nuclear DNA sequence.

27. The computer-implemented system of claim 24, wherein the plurality of genomes is at least 10,000 genomes.

28. The computer-implemented system of claim 24, wherein the nucleic acid sequence comprises at least 100,000 nucleotides.

29. The computer-implemented system of claim 24, comprising identifying the presence of at least 10 genomic sequence variants.

30. The computer-implemented system of claim 24, wherein the at least one genomic sequence variant comprises at least one of an insertion, a deletion, and a translocation.

31. The computer-implemented system of claim 24, wherein the at least one genomic sequence variant comprises a single nucleotide polymorphism.

32. The computer-implemented system of claim 24, wherein n equals 7.

33. The computer-implemented system of claim 24, wherein x is between 400 and 600.

34. The computer-implemented system of claim 24, comprising determining if the at least one genomic sequence variant is in a non-coding highly conserved genomic region.

35. The computer-implemented system of claim 34, the at least one genomic sequence variant is in a non-coding highly conserved genomic region within 2 megabases of a known disease-associated gene.

36. The computer-implemented system of claim 24, wherein the highly conserved genomic region is a genomic region corresponding to a most conserved 1^{st} percentile of all genomic regions.

37. The computer-implemented system of claim 24, wherein the observed context dependent tolerance score is at least 10% greater than an expected context dependent tolerance score.

38. The computer-implemented system of claim 24, wherein at least one of the at least one genomic sequence variant in a highly conserved genomic region is selected from the list consisting of rs587780751, rs745366624, rs777251123, rs778796405, rs774531501, rs587776927, rs768823171, rs749303140, rs376829288, rs750530042, rs587776558, rs372686280, rs111812550, rs143144732, rs193922699, rs750180293, rs398122808, rs757171524, rs773306994, rs773306994, rs372418954, rs762425885, rs397516031, rs397516022, rs730880592, rs730880592, rs397516020, rs397516020, rs373746463, rs373746463, rs373746463, rs387906397, rs387906397, rs587782958, rs730880718, rs730880667, rs113358486, rs111683277, rs112917345, rs730880691, rs397515916, rs730880690, rs111437311, rs397515903, rs727503201, rs112999777, rs397515897, rs727503204, rs397515893, rs397515891, rs587776699, rs587776700, rs376395543, rs748486465, rs149712664, rs199683937, rs144637717, rs587776644, rs730880296, rs397515322, rs558721552, rs531105836, rs587777262, rs267607302, rs387907354, rs398123750, rs727503988, rs587783714, rs148622862, rs763991428, rs761780097, rs770204470, rs387906521, rs387906520, rs79367981, rs749160734, rs587776708, rs587776708, rs34086577, rs199959804, rs587777290, rs386834170, rs386834169, rs144077391, rs386834164, rs386834166, rs770093080, rs587777374, rs45517105, rs45517105, rs45488500, rs45517289, rs45517289, rs137854118, rs45517358, rs189077405, rs515726118, rs386833742, rs386833739, rs755127868, rs200655247, rs376023420, rs747351687, rs113690956, rs376281637, rs765390290, rs773401248, rs61750189, rs530975087, rs201978571, rs267604791, rs80358116, rs80358116, rs273899695, rs80358011, rs80358011, rs80358051, rs730880267, rs63751296, rs63750707, rs776442328, rs776820510, rs72653165, rs72667012, rs72667008, rs527398797, rs587780009, rs587776658, rs587782018, rs745620135, rs372651309, rs556992558, rs137853932, rs200253809, rs386833901, rs770882876, rs750550558, rs397507554, rs730880306, rs201613240, rs147952488, rs770241629, rs373494631, rs397517741, rs386833856, rs559854357, rs371496308, rs539645405, rs187510057, rs41298629, rs536892777, rs747330606, rs748559929, rs770277446, rs201685922, rs767245071, rs730882032, rs587776525, rs398123358, rs72659359, rs137853943, rs267607709, rs267607710, rs766168993, rs775288140, rs780041521, rs145564018, rs775456047, rs587776879, rs540289812, rs745832717, rs745915863, rs386833418, rs199422309, rs431905514, rs587784059, rs748086984, rs386833492, rs199988476, rs281865166, rs587776515, rs397518439, rs193922258, rs142637046, rs73717525, rs145483167, rs587777285, rs747737281, rs183894680, rs116735828, rs574673404, rs386833563, rs768154316, rs111033661, rs755363896, rs368953604, rs180177319, rs148049120, rs150676454, rs372655486, rs373842615, rs763389916, rs118203419, rs515726232, rs312262809, rs312262804, rs281865349, rs281865338, rs281865337, rs281865334, rs281865336, rs281865336, rs62638626, rs62638627, rs587784423, rs113951193, rs281874765, rs104886349, rs398123247, rs74315277, rs200346587, rs398122908, rs727503036, rs397515747, and rs587776734.

39. The computer-implemented system of claim 24, wherein at least one of the at least one genomic sequence variant in a highly conserved genomic region is selected from the list consisting of rs778796405, rs8177982, rs376829288, rs4253196, rs750180293, rs757171524, rs727503201, rs397515893, rs587776699, rs397516083, rs201078659, rs750425291, rs558721552, rs531105836, rs200782636, rs752197734, rs3093266, rs34086577, rs199959804, rs144077391, rs386834164, rs386834166, rs189077405, rs746701685, rs386833721, rs376023420, rs761146008, rs765390290, rs72648337, rs527398797, rs367567416; rs372651309, rs200253809, rs193922837, rs761737358, rs113994173, rs559854357, rs111951711, rs371496308, rs368123079, rs118192239, rs41298629, and rs536892777.

40. The computer-implemented system of claim 24, wherein the functional genomic assay application is for use in determining a likelihood of the individual being diagnosed with a cancer.

41. The computer-implemented system of claim 24, wherein the functional genomic assay application is for use in prognosing a cancer of the individual.

42. The computer-implemented system of claim 24, wherein the functional genomic assay application is for use in determining a longevity of the individual.


SEQUENCE LISTING (35 sequences)

SEQ ID NO: 1 - DNA, 14 bp, Homo sapiens
cgcaccaacaacct

SEQ ID NO: 2 - DNA, 21 bp, Homo sapiens
ccatgccccgtgcttctggaa

SEQ ID NO: 3 - DNA, 10 bp, Homo sapiens
ccctttcccc

SEQ ID NO: 4 - DNA, 19 bp, Homo sapiens
attttagaccaatctgacc

SEQ ID NO: 5 - DNA, 14 bp, Homo sapiens
agggctacggcatg

SEQ ID NO: 6 - DNA, 12 bp, Homo sapiens
gggctacggcat

SEQ ID NO: 7 - DNA, 20 bp, Homo sapiens
aggcatgtcagccacgtggg

SEQ ID NO: 8 - DNA, 321 bp, Homo sapiens
cctgtggtcgagttggcctgcgtttcggatccgagggcgacgcagacggagctcagaaccagacccagccagagaaggcctcggccggtccggggtggcggcatttcggcttcgacgcggccgcttcagagcggcggggacaggctgcagcaggtggcgcagttagcagccgccgccgcagccacagagacctcctcgtcgggaacccatgaagactgcgcaacacagccgccgcccgggcccgcaggcccgggcgctggccgcagcgcgagtgcgtccgtgcgactcttccctgcgtccctcccctccggggcgggttct

SEQ ID NO: 9 - DNA, 35 bp, Homo sapiens
cgcgcgcgctagcgccgtgcgtgctgacggcatgt

SEQ ID NO: 10 - DNA, 14 bp, Homo sapiens
ctcaccagggaaag

SEQ ID NO: 11 - DNA, 10 bp, Homo sapiens
ggagagatta

SEQ ID NO: 12 - DNA, 12 bp, Homo sapiens
agcagacgggca

SEQ ID NO: 13 - DNA, 11 bp, Homo sapiens
accggccggcc

SEQ ID NO: 14 - DNA, 12 bp, Homo sapiens
acgcgcaagccg

SEQ ID NO: 15 - DNA, 14 bp, Homo sapiens
atgtttacgtcttc

SEQ ID NO: 16 - DNA, 12 bp, Homo sapiens
gcagctgtccct

SEQ ID NO: 17 - DNA, 15 bp, Homo sapiens
gcagctgtccctcac

SEQ ID NO: 18 - DNA, 12 bp, Homo sapiens
aagttttcccct

SEQ ID NO: 19 - DNA, 19 bp, Homo sapiens
atgaggaagaataagactc

SEQ ID NO: 20 - DNA, 19 bp, Homo sapiens
acaaaggggggtgttgtgg

SEQ ID NO: 21 - DNA, 61 bp, Homo sapiens
ctagccgtacggtaatctagccgtagactagccgtacggtaatatacaggtagactagccg

SEQ ID NO: 22 - DNA, 50 bp, Homo sapiens
ctagccgtacggtaatctagccgtagactagccgtacggtaatatattca

SEQ ID NO: 23 - protein, 21 aa, Homo sapiens
FERIKTLGTGSFGRVMLVKHK

SEQ ID NO: 24 - protein, 21 aa, Homo sapiens
FERKKTLGTGSFGRVMLVKHK

SEQ ID NO: 25 - protein, 21 aa, Homo sapiens
FERLRTLGMGSFGRVMLVRHQ

SEQ ID NO: 26 - protein, 21 aa, Homo sapiens
FNIIDTLGVGGFGRVELVQLK

SEQ ID NO: 27 - protein, 21 aa, Homo sapiens
LEIIATLGVGGFGRVELVKVK

SEQ ID NO: 28 - protein, 21 aa, Homo sapiens
FKFGKILGEGSFSTVVLAREL

SEQ ID NO: 29 - protein, 21 aa, Homo sapiens
FQILRAIGKGSFGKVCIVQKR

SEQ ID NO: 30 - protein, 21 aa, Homo sapiens
FEILRAIGKGSFGKVCIVQKN

SEQ ID NO: 31 - protein, 21 aa, Homo sapiens
FQILRAIGKGSFGKVCIVQKR

SEQ ID NO: 32 - protein, 21 aa, Homo sapiens
LKILGLVAKGSFGTVLKVLDC

SEQ ID NO: 33 - protein, 60 aa, Homo sapiens
CRVVGVIEKVQLVQDPATGGTFVVKSLPRCHMVSRERLTIIPHGVPYMTKLLRYFVSEDS

SEQ ID NO: 34 - protein, 16 aa, Homo sapiens
CSPLSGANEYIASTDT

SEQ ID NO: 35 - protein, 21 aa, Homo sapiens
FSIVKPISRGAFGKVYLGQKG